(19) United States
(12) Patent Application Publication
(10) Pub. No.: US 2018/0116976 A1
(43) Pub. Date: May 3, 2018

(54) METHODS AND COMPOSITIONS FOR UNWANTED OR ABNORMAL MUSCLE CONTRACTIONS

(71) Applicant: Flex Pharma, Inc., Boston, MA (US)

(72) Inventors: Christoph Westphal, Boston, MA (US); Jennifer Cermak, Berlin, MA (US); Thomas Wessel, Lenox, MA (US)

(21) Appl. No.: 15/819,881

(22) Filed: Nov. 21, 2017

Related U.S. Application Data
(63) Continuation of application No. PCT/US2016/055821, filed on Oct. 6, 2016.
(60) Provisional application No. 62/370,471, filed on Aug. 3, 2016.
(60) Provisional application No. 62/351,489, filed on Jun. 17, 2016.
(60) Provisional application No. 62/237,973, filed on Oct. 6, 2015.

(51) Int. Cl. A61K 31/12; A61K 9/00; A61P 21/02

(52) U.S. Cl. CPC A61K 31/12; A61P 21/02; A61K 9/0056

(57) ABSTRACT

The invention relates to methods and compositions for treating muscle cramps, muscle spasms, muscle spasticity, dystonias, and fasciculations.

### FIELD OF INVENTION

The invention relates to methods and compositions for evaluating and treating muscle cramps, muscle spasms, muscle spasticity, dystonias, and fasciculations.

### BACKGROUND

Unwanted or abnormal muscle contractions are often painful and can last for a prolonged period of time. These contractions can be triggered by exercise or may also occur spontaneously (e.g., muscle spasms or nocturnal leg cramps). The underlying physiological mechanism of unwanted or abnormal muscle contractions is unknown. Recent understanding has led to the hypothesis that these muscle contractions, such as muscle cramps and muscle spasms, result from excessive electrical firing of the alpha motor neurons that project from the spinal cord and trigger contraction of skeletal muscles (Schwellnus, M. P. Br J Sports Med (2009) 43:401-408; Miller, K. C. et al, Med Sci Sports Exerc (2010) 42:953-61). Studies have revealed that modulating the negative feedback regulation in the alpha motor neuron circuit by stimulating primary sensory neurons to send inhibitory signals to the alpha motor neurons may be effective for reducing or inhibiting unwanted or abnormal muscle contractions. In addition, unwanted or abnormal muscle contractions, such as muscle cramps, spasms, dystonias, or fasciculations, are also experienced by patients that have neuromuscular diseases, respiratory conditions, and other disorders.

Currently, few effective treatments and therapeutic regimens are available to alleviate unwanted or abnormal muscle contractions, e.g., muscle cramps, spasms, dystonias, fasciculations, and the like. As such, there exists a need in the art for improved methods and compositions for preventing, treating, and ameliorating these unwanted or abnormal muscle contractions. Furthermore, there exists a need for diagnostic methods for identifying or classifying subjects who will be responsive to spasm or cramp therapies.

### SUMMARY OF THE INVENTION

In one aspect, the present disclosure features a method for preventing or treating an unwanted or abnormal muscle contraction in a subject, the method comprising orally administering an effective amount of a composition to the subject, wherein the composition comprises a single gingerol analog and less than about 25% of the total gingerol concentration in the composition (e.g., by mole or by weight) of a related analog thereof (e.g., less than about 20%, less than about 15%, less than about 10%, less than about 5%, less than about 3%, less than about 1%, less than about 0.1%, or is substantially free of a related analog thereof). In an embodiment, the composition comprises a single gingerol analog and is substantially free of a related analog thereof.

In some embodiments, the single gingerol analog is, e.g., 6-gingerol. In some embodiments, the amount of the single gingerol analog (e.g., 6-gingerol) in the composition is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single gingerol analog (e.g., 6-gingerol) in the composition is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single gingerol analog (e.g. 6-gingerol) in the composition is from about 0.0001% (w/v) or (w/w) to about 10% (w/v) or (w/w). In some embodiments, the amount of the single gingerol analog (e.g. 6-gingerol) in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In some embodiments, the composition further comprises a single shogaol analog, e.g., 6-shogaol. In some embodiments, the amount of the single shogaol analog (e.g., 6-shogaol) in the composition is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single shogaol analog (e.g., 6-shogaol) in the composition is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single shogaol analog (e.g. 6-shogaol) in the composition is from about 0.0001% (w/v) or (w/w) to about 10% (w/v) or (w/w). In some embodiments, the amount of the single shogaol analog (e.g. 6-shogaol) in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In another aspect, the present disclosure features a method for preventing or treating an unwanted or abnormal muscle contraction in a subject, the method comprising orally administering an effective amount of a composition to the subject, wherein the composition comprises a single shogaol analog and less than about 25% of the total shogaol concentration in the composition (e.g., by mole or by weight) of a related analog thereof (e.g., less than about 20%, less than about 15%, less than about 10%, less than about 5%, less than about 3%, less than about 1%, less than about 0.1%, or is substantially free of a related analog thereof). In an embodiment, the composition comprises a single shogaol analog and is substantially free of a related analog thereof.

In some embodiments, the single shogaol analog is, e.g., 6-shogaol. In some embodiments, the amount of the single shogaol analog (e.g., 6-shogaol) in the composition is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single shogaol analog (e.g., 6-shogaol) in the composition is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single shogaol analog (e.g. 6-shogaol) in the composition is from about 0.0001% (w/v) or (w/w) to about 10% (w/v) or (w/w). In some embodiments, the amount of the single shogaol analog (e.g. 6-shogaol) in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In some embodiments, the composition further comprises a single gingerol analog, e.g., 6-gingerol. In some embodiments, the amount of the single gingerol analog (e.g., 6-gingerol) in the composition is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single gingerol analog (e.g., 6-gingerol) in the composition is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single gingerol analog (e.g. 6-gingerol) in the composition is from about 0.0001% (w/v) or (w/w) to about 10% (w/v) or (w/w). In some embodiments, the amount of the single gingerol analog (e.g. 6-gingerol) in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In any and all aspects, in some embodiments, the molar ratio of the single gingerol analog (e.g., 6-gingerol) and the single shogaol analog (e.g., 6-shogaol) is from about 1000:1 to about 1:1 (e.g., about 750:1, about 500:1, about 250:1, about 200:1, about 100:1, about 50:1, about 25:1, about 10:1, about 5:1, about 2.5:1).

In some embodiments, the composition further comprises a single capsaicinoid analog, e.g., capsaicin. In some embodiments, the amount of the single capsaicinoid analog (e.g., capsaicin) in the composition is from 0.001 mg to about 20 mg (e.g., about 0.01 mg to about 10 mg, about 0.05 mg to about 5 mg, about 0.075 mg to about 2.5 mg, about 0.1 mg to about 1 mg). In some embodiments, the amount of the single capsaicinoid analog (e.g. capsaicin) in the composition is from about 0.0001% (w/v) or (w/w) to about 10% (w/v) or (w/w). In some embodiments, the amount of the single capsaicinoid analog (e.g. capsaicin) in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single gingerol analog (e.g., 6-gingerol) and the single capsaicinoid analog (e.g., capsaicin) is from about 1000:1 to about 5:1 (e.g., about 750:1, about 500:1, about 250:1, about 200:1, about 100:1, about 50:1, about 25:1, about 10:1). In some embodiments, the molar ratio of the single shogaol analog (e.g., 6-shogaol) and the single capsaicinoid analog (e.g., capsaicin) is from about 1000:1 to about 5:1 (e.g., about 750:1, about 500:1, about 250:1, about 200:1, about 100:1, about 50:1, about 25:1, about 10:1).

In some embodiments, the composition further comprises trans-cinnamaldehyde. In some embodiments, the amount of the trans-cinnamaldehyde in the composition is from 5 mg to about 1000 mg (e.g., about 10 mg to about 250 mg, about 15 mg to about 200 mg, about 20 mg to about 100 mg, about 25 mg to about 75 mg). In some embodiments, the amount of trans-cinnamaldehyde in the composition is from about 0.0001% (w/v) or (w/w) to about 20% (w/v) or (w/w). In some embodiments, the amount of trans-cinnamaldehyde in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single gingerol analog (e.g., 6-gingerol) and trans-cinnamaldehyde is from about 1000:1 to about 1:1 (e.g., about 750:1, about 500:1, about 250:1, about 200:1, about 100:1, about 50:1, about 25:1, about 10:1, about 5:1, about 2.5:1). In some embodiments, the molar ratio of the single shogaol analog (e.g., 6-shogaol) and trans-cinnamaldehyde is from about 1000:1 to about 1:1 (e.g., about 750:1, about 500:1, about 250:1, about 200:1, about 100:1, about 50:1, about 25:1, about 10:1, about 5:1, about 2.5:1).

In some embodiments, the composition has a residence time of greater than about 5 seconds in the mouth of a subject, e.g., greater than about 6 seconds, about 7 seconds, about 8 seconds, about 9 seconds, about 10 seconds, about 11 seconds, about 12 seconds, about 13 seconds, about 14 seconds, about 15 seconds, about 20 seconds, about 25 seconds, about 30 seconds, about 45 seconds, about 60 seconds, about 90 seconds, about 2 minutes, about 3 minutes, about 4 minutes, about 5 minutes, or more. In some embodiments, the composition has a residence time of greater than about 60 seconds in the mouth of a subject, e.g., greater than about 90 seconds, about 2 minutes, about 3 minutes, about 4 minutes, about 5 minutes, about 6 minutes, about 7 minutes, about 8 minutes, about 9 minutes, about 10 minutes, or more.

In some embodiments, the composition is formulated to have minimal systemic exposure in the subject, e.g., wherein the systemic exposure is measured through the concentration of the active ingredient (e.g., a gingerol, e.g., 6-gingerol) in the blood, urine, or tissue (e.g., adipose tissue) of the subject. In some embodiments, the composition is formulated to have less than about 50% total systemic exposure in a subject, e.g., less than about 40%, less than about 30%, less than about 20%, less than about 10%, less than about 5%, less than about 1%, less than about 0.5%, less than about 0.1%, less than about 0.05%, less than about 0.01%, or less systemic exposure in a subject.

In another aspect, the present disclosure features a method for preventing or treating an unwanted or abnormal muscle contraction in a subject, the method comprising orally administering an effective amount of a composition to the subject, wherein the composition comprises a single capsaicinoid analog and less than about 25% of the total capsaicinoid concentration in the composition (e.g., by mole or by weight) of a related analog thereof (e.g., less than about 20%, less than about 15%, less than about 10%, less than about 5%, less than about 3%, less than about 1%, less than about 0.1%, or is substantially free of a related analog thereof). In an embodiment, the composition comprises a single capsaicinoid analog and is substantially free of a related analog thereof.

In some embodiments, the single capsaicinoid analog is, e.g., capsaicin. In some embodiments, the amount of the single capsaicinoid analog (e.g., capsaicin) in the composition is from 0.001 mg to about 20 mg (e.g., about 0.01 mg to about 10 mg, about 0.05 mg to about 5 mg, about 0.075 mg to about 2.5 mg, about 0.1 mg to about 1 mg). In some embodiments, the amount of the single capsaicinoid analog (e.g. capsaicin) in the composition is from about 0.0001% (w/v) or (w/w) to about 10% (w/v) or (w/w). In some embodiments, the amount of the single capsaicinoid analog (e.g. capsaicin) in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In some embodiments, the composition further comprises a single gingerol analog, e.g., 6-gingerol. In some embodiments, the amount of the single gingerol analog (e.g., 6-gingerol) in the composition is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single gingerol analog (e.g., 6-gingerol) in the composition is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single gingerol analog (e.g. 6-gingerol) in the composition is from about 0.0001% (w/v) or (w/w) to about 10% (w/v) or (w/w). In some embodiments, the amount of the single gingerol analog (e.g. 6-gingerol) in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single capsaicinoid analog (e.g., capsaicin) and the single gingerol analog (e.g., 6-gingerol) is from about 1000:1 to about 1:1 (e.g., about 750:1, about 500:1, about 250:1, about 200:1, about 100:1, about 50:1, about 25:1, about 10:1, about 5:1, about 2.5:1).

In some embodiments, the composition further comprises a single shogaol analog, e.g., 6-shogaol. In some embodiments, the amount of the single shogaol analog (e.g., 6-shogaol) in the composition is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single shogaol analog (e.g., 6-shogaol) in the composition is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single shogaol analog (e.g. 6-shogaol) in the composition is from about 0.0001% (w/v) or (w/w) to about 10% (w/v) or (w/w). In some embodiments, the amount of the single shogaol analog (e.g. 6-shogaol) in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single capsaicinoid analog (e.g., capsaicin) and the single shogaol analog (e.g., 6-shogaol) is from about 1000:1 to about 1:1 (e.g., about 750:1, about 500:1, about 250:1, about 200:1, about 100:1, about 50:1, about 25:1, about 10:1, about 5:1, about 2.5:1).

In some embodiments, the composition further comprises trans-cinnamaldehyde. In some embodiments, the amount of the trans-cinnamaldehyde in the composition is from 5 mg to about 1000 mg (e.g., about 10 mg to about 250 mg, about 15 mg to about 200 mg, about 20 mg to about 100 mg, about 25 mg to about 75 mg). In some embodiments, the amount of trans-cinnamaldehyde in the composition is from about 0.0001% (w/v) or (w/w) to about 20% (w/v) or (w/w). In some embodiments, the amount of trans-cinnamaldehyde in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single capsaicinoid analog (e.g., capsaicin) and trans-cinnamaldehyde is from about 1000:1 to about 1:1 (e.g., about 750:1, about 500:1, about 250:1, about 200:1, about 100:1, about 50:1, about 25:1, about 10:1, about 5:1, about 2.5:1).

In some embodiments, the composition has a residence time of greater than about 5 seconds in the mouth of a subject, e.g., greater than about 6 seconds, about 7 seconds, about 8 seconds, about 9 seconds, about 10 seconds, about 11 seconds, about 12 seconds, about 13 seconds, about 14 seconds, about 15 seconds, about 20 seconds, about 25 seconds, about 30 seconds, about 45 seconds, about 60 seconds, about 90 seconds, about 2 minutes, about 3 minutes, about 4 minutes, about 5 minutes, or more. In some embodiments, the composition has a residence time of greater than about 60 seconds in the mouth of a subject, e.g., greater than about 90 seconds, about 2 minutes, about 3 minutes, about 4 minutes, about 5 minutes, about 6 minutes, about 7 minutes, about 8 minutes, about 9 minutes, about 10 minutes, or more.

In some embodiments, the composition is formulated to have minimal systemic exposure in the subject, e.g., wherein the systemic exposure is measured through the concentration of the active ingredient (e.g., a capsaicinoid, e.g., capsaicin) in the blood, urine, or tissue (e.g., adipose tissue) of the subject. In some embodiments, the composition is formulated to have less than about 50% total systemic exposure in a subject, e.g., less than about 40%, less than about 30%, less than about 20%, less than about 10%, less than about 5%, less than about 1%, less than about 0.5%, less than about 0.1%, less than about 0.05%, less than about 0.01%, or less systemic exposure in a subject.

In another aspect, the present disclosure features a method for preventing or treating an unwanted or abnormal muscle contraction in a subject, the method comprising orally administering an effective amount of a composition to the subject, wherein the composition comprises a single capsaicinoid analog (e.g., capsaicin) and one of a single gingerol analog (e.g., 6-gingerol) or a single shogaol analog (e.g., 6-shogaol), and wherein said composition is substantially free of a related analog thereof. In some embodiments, the composition comprises a single capsaicinoid analog (e.g., capsaicin) and one of a single gingerol analog (e.g., 6-gingerol) or a single shogaol analog (e.g., 6-shogaol), and less than about 25% of the total capsaicinoid, gingerol, or shogaol concentration in the composition (e.g., by mole or by weight) of related analogs thereof (e.g., less than about 20%, less than about 15%, less than about 10%, less than about 5%, less than about 3%, less than about 1%, less than about 0.1%, or is substantially free of a related analog thereof).

In another aspect, the present disclosure features a method for the prevention or treatment of unwanted or abnormal contractions or absence of normal muscle contractions, wherein the method comprises orally administering a composition comprising a dosage of a single gingerol analog (e.g., 6-gingerol), a single shogaol analog (e.g., 6-shogaol), or a single capsaicinoid analog (e.g., capsaicin), wherein if present, the dosage of the single gingerol analog (e.g., 6-gingerol) or single shogaol analog (e.g., 6-shogaol) in the composition is between about 0.01 mg and about 1000 mg; and if present, the dosage of the single capsaicinoid analog (e.g., capsaicin) in the composition is between about 0.001 mg and about 100 mg. In an embodiment, the composition comprises a single gingerol analog (e.g., 6-gingerol), a single shogaol analog (e.g., 6-shogaol), or a single capsaicinoid analog (e.g., capsaicin), and is substantially free of related analogs thereof.

In another aspect, the present disclosure features a method for the prevention or treatment of unwanted or abnormal contractions or absence of normal muscle contractions, wherein the method comprises orally administering a composition comprising a dosage of a single gingerol analog (e.g., 6-gingerol), a single shogaol analog (e.g., 6-shogaol), or a single capsaicinoid analog (e.g., capsaicin), wherein if present, the dosage of the single gingerol analog (e.g., 6-gingerol) or single shogaol analog (e.g., 6-shogaol) in the composition is between about 0.05 mM and about 500 mM; and if present, the dosage of the single capsaicinoid analog (e.g., capsaicin) in the composition is between about 0.05 μM and about 100 μM. In an embodiment, the composition comprises a single gingerol analog (e.g., 6-gingerol), a single shogaol analog (e.g., 6-shogaol), or a single capsaicinoid analog (e.g., capsaicin), and is substantially free of related analogs thereof.

In another aspect, the present disclosure features a method for the prevention or treatment of unwanted or abnormal contractions or absence of normal muscle contractions, wherein the method comprises orally administering a composition comprising a single gingerol analog (e.g., 6-gingerol), a single shogaol analog (e.g., 6-shogaol), or a single capsaicinoid analog (e.g., capsaicin), wherein if present, the dosage of the single gingerol analog (e.g., 6-gingerol) or single shogaol analog (e.g., 6-shogaol) in the composition is between about 0.001% (w/v) or (w/w) and about 10% (w/v) or (w/w) or between about 0.01% (w/v) or (w/w) and about 50% (w/v) or (w/w); and if present, the dosage the single capsaicinoid analog (e.g., capsaicin) in the composition is between about 0.0001% (w/v) or (w/w) and about 5% (w/v) or (w/w) or between about 0.01% (w/v) or (w/w) and about 10% (w/v) or (w/w). In an embodiment, the composition comprises a single gingerol analog (e.g., 6-gingerol), a single shogaol analog (e.g., 6-shogaol), or a single capsaicinoid analog (e.g., capsaicin), and is substantially free of related analogs thereof.

In another aspect, the present disclosure features a method for the prevention or treatment of unwanted or abnormal contractions or absence of normal muscle contractions, wherein the method comprises orally administering a composition comprising a single capsaicinoid analog (e.g., capsaicin) and one of a single gingerol analog (e.g., 6-gingerol) or a single shogaol analog (e.g., 6-shogaol), wherein the dosage of the single gingerol analog (e.g., 6-gingerol) or the single shogaol analog (e.g., 6-shogaol) in the composition is between about 0.01 mg and about 1000 mg; and the dosage of the single capsaicinoid analog (e.g., capsaicin) in the composition is between about 0.001 mg and about 100 mg. In an embodiment, the composition comprises a single gingerol analog (e.g., 6-gingerol) or a single shogaol analog (e.g., 6-shogaol) and a single capsaicinoid analog (e.g., capsaicin), and is substantially free of related analogs thereof.

In another aspect, the present disclosure features a method for preventing or treating an unwanted or abnormal muscle contraction in a subject, the method comprising orally administering an effective amount of a composition to the subject, wherein the composition comprises a single gingerol analog and is administered in a dosage sufficient to prevent, diminish the severity of, or reduce the frequency of an unwanted or abnormal muscle contraction in the subject. In an embodiment, the composition comprises a single gingerol analog (e.g., 6-gingerol) and is substantially free of related analogs thereof.

In another aspect, the present disclosure features a method for preventing or treating an unwanted or abnormal muscle contraction in a subject, the method comprising orally administering an effective amount of a composition to the subject, wherein the composition comprises a single shogaol analog and is administered in a dosage sufficient to prevent, diminish the severity of, or reduce the frequency of an unwanted or abnormal muscle contraction in the subject. In an embodiment, the composition comprises a single shogaol analog (e.g., 6-shogaol) and is substantially free of related analogs thereof.

In another aspect, the present disclosure features a method for preventing or treating an unwanted or abnormal muscle contraction in a subject, the method comprising orally administering an effective amount of a composition to the subject, wherein the composition comprises a single capsaicinoid analog and is administered in a dosage sufficient to prevent, diminish the severity of, or reduce the frequency of an unwanted or abnormal muscle contraction in the subject. In an embodiment, the composition comprises a single capsaicinoid analog (e.g., capsaicin) and is substantially free of related analogs thereof.

In another aspect, the present disclosure features a method for preventing or treating an unwanted or abnormal muscle contraction in a subject, the method comprising orally administering an effective amount of a composition to the subject, wherein the composition comprises a single capsaicinoid analog and one of a single gingerol analog or a single shogaol analog, and is administered in a dosage sufficient to prevent, diminish the severity of, or reduce the frequency of an unwanted or abnormal muscle contraction in the subject. In an embodiment, the composition comprises a single gingerol analog (e.g., 6-gingerol) or a single shogaol analog (e.g., 6-shogaol) and a single capsaicinoid analog (e.g., capsaicin), and is substantially free of related analogs thereof.

In another aspect, the present disclosure features a method for preventing or treating an unwanted or abnormal muscle contraction in a subject, the method comprising orally administering an effective amount of a composition to the subject, wherein the composition comprises a single gingerol analog and is administered daily (e.g., once, twice, or three times daily), e.g., in an amount sufficient to prevent, diminish the severity of, or reduce the frequency of the unwanted or abnormal muscle contractions in the subject. In an embodiment, the composition comprises a single gingerol analog (e.g., 6-gingerol) and is substantially free of related analogs thereof.

In another aspect, the present disclosure features a method for preventing or treating an unwanted or abnormal muscle contraction in a subject, the method comprising orally administering an effective amount of a composition to the subject, wherein the composition comprises a single shogaol analog (e.g., 6-shogaol) and is administered daily (e.g., once, twice, or three times daily), e.g., in an amount sufficient to prevent, diminish the severity of, or reduce the frequency of the unwanted or abnormal muscle contractions in the subject. In an embodiment, the composition comprises a single shogaol analog (e.g., 6-shogaol) and is substantially free of related analogs thereof.

In another aspect, the present disclosure features a method for preventing or treating an unwanted or abnormal muscle contraction in a subject, the method comprising orally administering an effective amount of a composition to the subject, wherein the composition comprises a single capsaicinoid analog (e.g., capsaicin) and is administered daily (e.g., once, twice, or three times daily), e.g., in an amount sufficient to prevent, diminish the severity of, or reduce the frequency of the unwanted or abnormal muscle contractions in the subject. In an embodiment, the composition comprises a single capsaicinoid analog (e.g., capsaicin) and is substantially free of related analogs thereof.

In another aspect, the present disclosure features a method for preventing or treating an unwanted or abnormal muscle contraction in a subject, the method comprising orally administering an effective amount of a composition to the subject, wherein the composition comprises a single capsaicinoid analog and one of a single gingerol analog or a single shogaol analog, and the composition is administered daily (e.g., once, twice, or three times daily), e.g., in an amount sufficient to prevent, diminish the severity of, or reduce the frequency of the unwanted or abnormal muscle contractions in the subject. In an embodiment, the composition comprises a single gingerol analog (e.g., 6-gingerol) or a single shogaol analog (e.g., 6-shogaol) and a single capsaicinoid analog (e.g., capsaicin), and is substantially free of related analogs thereof.

In another aspect, the present disclosure features a method of evaluating a subject for an unwanted or abnormal muscle contraction or absence of normal muscle contraction in a subject comprising: a) acquiring, e.g., indirectly or directly, knowledge of a result of a test for the efficacy of the administration of a test aliquot of a composition comprising a single gingerol analog, a single shogaol analog, or a single capsaicinoid analog to the subject that is substantially free of related analogs thereof; and b) administering, e.g., in response to said result, an amount of said composition sufficient to alleviate an unwanted or abnormal muscle contraction or absence of normal muscle contraction to said subject. In an embodiment, the composition comprises a single gingerol analog (e.g., 6-gingerol), a single shogaol analog (e.g., 6-shogaol), or a single capsaicinoid analog (e.g., capsaicin), and is substantially free of related analogs thereof.

In any of the above-mentioned aspects, in some embodiments, the methods and compositions may comprise any of the features described below. In some embodiments, the unwanted or abnormal muscle contraction comprises a muscle cramp (e.g., a night cramp). In some embodiments, the muscle contraction comprises a muscle spasm. In some embodiments, the muscle contraction comprises an overall increase in muscle tone, or spasticity. In some embodiments, the muscle contraction comprises a dystonia (e.g., a cervical dystonia). In some embodiments, the muscle contraction comprises a fasciculation. In some embodiments, the muscle contraction occurs in a skeletal muscle. In some embodiments, the muscle contraction occurs in a smooth muscle. In some embodiments, the subject has a central nervous system disorder or injury, e.g., a brain injury, stroke, or traumatic spinal cord injury.

In some embodiments, the subject has been diagnosed with or identified as having multiple sclerosis. In some embodiments, the subject has been diagnosed with or identified as having multiple sclerosis and is further suffering from spasticity, spasms, or cramps. A subject may be diagnosed or identified as having multiple sclerosis by any method known in the art, e.g., through determination of scoring on the Clinical Global Impression (CGI) Scale or on a Numerical Rating Scale (NRS). In some embodiments, the subject is suffering from spasticity, spasms, or cramps related to multiple sclerosis and the composition is administered to the subject daily (e.g., once, twice, or three times daily) in an amount sufficient to prevent, diminish the severity of, or reduce the frequency of said spasticity, spasms, or cramps, e.g., as measured by the Modified Ashworth Scale, the Tardieu Scale, a Numerical Rating Scale (e.g., a Numerical Rating Scale for Spasticity), the Barthel Activities of Daily Living (ADL) Scale, the Clinical Global Impression (CGI) Scale, or Quality of Life questionnaires (e.g., the 36-Item Short Form Survery (SF-36) and Multiple Sclerosis Spasticity Scale (MSSS-88). In some embodiments, the spasticity, spasms, or cramps related to multiple sclerosis are reduced by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more, e.g., as measured by the Modified Ashworth Scale, the Tardieu Scale, or a Numerical Rating Scale (e.g., a Numerical Rating Scale for Spasticity).

In some embodiments, the subject is suffering from spasticity, spasms, or cramps related to multiple sclerosis and the composition is administered to the subject daily (e.g., once, twice, or three times daily) in an amount sufficient to improve the gait of the subject, e.g., as measured by the Timed 25-Foot Walk Test. In some embodiments, the improvement of the gait comprises a faster walking speed, faster cadence, or longer stride length, e.g., as measured by the Timed 25-Foot Walk Test. In some embodiments, the gait of the subject suffering from spasticity, spasms, or cramps related to multiple sclerosis is improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more, e.g., as measured by the Timed 25-Foot Walk Test.

In some embodiments, the subject is suffering from spasticity, spasms, or cramps related to multiple sclerosis and the composition is administered to the subject daily (e.g., once, twice, or three times daily) in an amount sufficient to improve the quality of sleep, e.g., as measured by the Insomnia Severity Index Sleep Survey, the Epworth Sleepiness Scale, or the Medical Outcomes Study-Sleep Scale. In some embodiments, the quality of sleep of the subject suffering from spasticity, spasms, or cramps related to multiple sclerosis is improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more, e.g., as measured by the Insomnia Severity Index Sleep Survey, the Epworth Sleepiness Scale, or the Medical Outcomes Study-Sleep Scale.

In some embodiments, the subject has been diagnosed with or identified as having dystonia, e.g., focal dystonia, blepharospasm, cervical dystonia, cranial dystonia, laryngeal dystonia, and hand dystonia. In some embodiments, the subject has been diagnosed with or identified as having cervical dystonia. In some embodiments, the subject is suffering from cervical dystonia and the composition is administered to the subject daily (e.g., once, twice, or three times daily) in an amount sufficient to improve the amplitude of movement of the head or neck relative to the amplitude of movement of the head or neck prior to treatment, e.g., as measured by the Toronto Western Spasmodic Torticollis Rating Scale, the Tsui score, or the Oropharyngeal Swallow Efficiency Test. In some embodiments, the amplitude of movement of the head or neck of the subject suffering from cervical dystonia is improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more, e.g., as measured by the Toronto Western Spasmodic Torticollis Rating Scale, the Tsui score, or the Oropharyngeal Swallow Efficiency Test.

In some embodiments, the subject is suffering from cervical dystonia and the composition is administered to the subject daily (e.g., once, twice, or three times daily) in an amount sufficient to prevent, diminish the severity of, or reduce the frequency of pain relating to said cervical dystonia, e.g., as measured by the Toronto Western Spasmodic Torticollis Rating Scale, the Tsui score, or the Oropharyngeal Swallow Efficiency Test. In some embodiments, the pain of the subject suffering from cervical dystonia is improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more, e.g., as measured by the Toronto Western Spasmodic Torticollis Rating Scale, the Tsui score, or the Oropharyngeal Swallow Efficiency Test.

In some embodiments, the subject has been diagnosed with or identified as having spinal cord spasticity. In some embodiments, the subject has been diagnosed with or identified as having spasticity due to spinal cord injury. In some embodiments, the subject has been diagnosed with or identified as having a muscle condition or disorder, e.g., any of the disorders disclosed herein, e.g., nocturnal cramps, multiple sclerosis, spinal cord spasticity, or dystonia (e.g., cervical dystonia).

In some embodiments, the subject has been diagnosed or identified as having nocturnal cramps (e.g., nocturnal leg cramps or nocturnal foot cramps). A subject may be diagnosed or identified as having nocturnal cramps by any method known in the art, e.g., through determination of whether the subject meets minimal American Academy of Sleep Medicine (AASM) criteria for nocturnal cramps. In some embodiments, the subject is suffering from nocturnal cramps (e.g., nocturnal leg cramps or nocturnal foot cramps) and the composition is administered to the subject daily (e.g., once, twice, or three times daily) in an amount sufficient to prevent, diminish the severity of, or reduce the frequency of the nocturnal leg cramps in the subject, e.g., as measured by cramp frequency, cramp severity, cramp-free nights, and/or cramp-free days, and/or as assessed by a Visual Analogue Scale, a Numerical Rating Scale for Pain, the Clinical Global Impression of Change (CGI-C) Scale, or Patient Global Impression of Change (PGI-C) Scale. In some embodiments, the frequency or severity of the nocturnal cramps (e.g., nocturnal leg cramps or nocturnal foot cramps) in the subject is reduced by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more, e.g., as measured by cramp frequency, cramp severity, number of cramp-free nights, and/or number of cramp-free days, and/or as assessed by a Visual Analogue Scale, a Numerical Rating Scale for Pain, or by the Clinical Global Impression of Change (CGI-C) Scale, or and/or Patient Global Impression Scale. In some embodiments, the nocturnal cramps (e.g., nocturnal leg cramps or nocturnal foot cramps) result from an underlying disease or disorder (e.g., periodic limb movement disorder). In some embodiments, the nocturnal cramps (e.g., nocturnal leg cramps or nocturnal foot cramps) are not the result of another disease or disorder, e.g., and are present in an otherwise healthy individual. In some embodiments, the nocturnal cramps (e.g., nocturnal leg cramps or nocturnal foot cramps) cause a disruption (e.g., a meaningful disruption) of the daily functioning of a subject. In some embodiments, the methods described herein improve the daily functioning of a subject, e.g., a subject having nocturnal cramps (e.g., nocturnal leg cramps or nocturnal foot cramps).

In some embodiments, the subject is suffering from nocturnal cramps (e.g., nocturnal leg cramps or nocturnal foot cramps) and the composition is administered to the subject daily (e.g., once, twice, or three times daily) in an amount sufficient to improve the quality of sleep, e.g., as measured by the Insomnia Severity Index Sleep Survey, the Epworth Sleepiness Scale, or the Medical Outcomes Study-Sleep Scale. In some embodiments, the quality of sleep of the subject suffering from nocturnal cramps (e.g., nocturnal leg cramps or nocturnal foot cramps) is improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more, e.g., as measured by the Insomnia Severity Index Sleep Survey, the Epworth Sleepiness Scale, or the Medical Outcomes Study-Sleep Scale. In some embodiments, the nocturnal cramps (e.g., nocturnal leg cramps or nocturnal foot cramps) result from an underlying disease or disorder (e.g., periodic limb movement disorder). In some embodiments, the nocturnal cramps (e.g., nocturnal leg cramps or nocturnal foot cramps) are not the result of another disease or disorder, e.g., and are present in an otherwise healthy individual. In some embodiments, the nocturnal cramps (e.g., nocturnal leg cramps or nocturnal foot cramps) cause a disruption (e.g., a meaningful disruption) of daily functioning of a subject. In some embodiments, the methods described herein improve the daily functioning of a subject, e.g., a subject having nocturnal cramps (e.g., nocturnal leg cramps or nocturnal foot cramps).

In some embodiments, the muscle contraction comprises a contraction in a muscle of the foot, e.g., the flexor hallucis brevis muscle. In some embodiments, the muscle contraction is associated with multiple sclerosis, and e.g., comprises a contraction in the muscles of the arm or leg. In some embodiments, the muscle contraction is associated with spinal cord spasticity, and e.g., comprises a contraction in the muscles of the neck or back. In some embodiments, the muscle contraction is associated with dystonia, and e.g., comprises a contraction in the muscles of the neck, hand, arm, or foot.

In some embodiments, the muscle contraction is associated with a neurological condition (e.g., a peripheral nervous system condition or a central nervous system condition). In some embodiments, the muscle contraction is associated with a peripheral nervous system condition, e.g., cramp fasciculation syndrome, Isaacs' Syndrome or neuromyotonia, peripheral neuropathy, peripheral nerve hyperexcitability disorder, carpal tunnel syndrome, or Epstein-Barr virus infection). In some embodiments, the muscle contraction is associated with a central nervous system condition, e.g., multiple sclerosis, cerebral palsy, stroke, traumatic brain injury, spinal injury (e.g., spinal cord injury), stenosis (e.g., spinal stenosis), motor neuron disease, or a central nervous system tumor (e.g., a brain or spinal cord tumor). In some embodiments, the muscle contraction is associated with a throat condition, e.g. acid reflux, laryngospasm, dysphagia, spasmodic dysphonias or the throat condition is associated with chemical injury, cancer, surgical injury, or pathogen infection. In some embodiments, the muscle contraction is associated with an electrolyte imbalance or vitamin deficiency, e.g., hyponatremia, hypocalcemia, hypomagnesemia, kidney disease, rickets, calcium or magnesium deficiency, thiamine deficiency, hypoparathyroidism, medullary cystic disease, or adrenocortical carcinoma.

In some embodiments, the muscle contraction is associated with a motor neuron disease, e.g., amyotrophic lateral sclerosis, primary lateral sclerosis, progressive muscular atrophy, progressive bulbar palsy, pseudobulbar palsy, spinal muscular atrophy, progressive spinobulbar muscular atrophy, or post-polio syndrome. In some embodiments, the subject is suffering from muscle cramps or spasms related to a motor neuron disease or a peripheral nerve hyperexcitability disorder and the composition is administered to the subject daily (e.g., once, twice, or three times daily) in an amount sufficient to prevent, diminish the severity of, or reduce the frequency of said muscle cramps or spasms in the subject, e.g., as measured by the pain and intensity of cramps, the ALS Assessment Questionnaire, a Numerical Rating Scale (e.g., the Numerical Rating Scale for spasticity), the Modified Ashworth Scale, the Tardieu Scale, the Patient Global Impression of Change scale, Clinical Global Impression scale, or the Insomnia and Severity Index (ISIS) Survey. In some embodiments, the frequency or severity of the muscle cramps or spasms related to a motor neuron disease or a peripheral nerve hyperexcitability disorder in the subject is reduced by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more, e.g., as measured by the pain and intensity of cramps, the ALS Assessment Questionnaire, a Numerical Rating Scale (e.g., the Numerical Rating Scale for spasticity), the Modified Ashworth Scale, the Tardieu Scale, the Patient Global Impression of Change scale, Clinical Global Impression scale, or the Insomnia and Severity Index (ISIS) Survey.

In some embodiments, the subject is suffering from muscle cramps or spasms related to a motor neuron disease or a peripheral nerve hyperexcitability disorder and the composition is administered to the subject daily (e.g., once, twice, or three times daily) in an amount sufficient to improve the quality of sleep, e.g., as measured by the Insomnia Severity Index Sleep Survey or the Epworth Sleepiness Scale. In some embodiments, the quality of sleep of the subject suffering from muscle cramps or spasms related to a motor neuron disease or a peripheral nerve hyperexcitability disorder is improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more, e.g., as measured by the Insomnia Severity Index Sleep Survey or the Epworth Sleepiness Scale.

In some embodiments, the muscle contraction is associated with a respiratory condition, e.g., asthma, chronic obstructive pulmonary disease, bronchitis, emphysema, pneumonia, cystic fibrosis, pleural cavity diseases, influenza, or cold. In some embodiments, the muscle contraction is associated with a connective tissue disease, e.g., Ehlers-Danlos syndrome, epidermolysis bullosa, Marfan syndrome, osteogenesis imperfect, arthritis, scleroderma, Sjögren's syndrome, lupus, vasculitis, mixed connective tissue disease, cellulitis, polymyositis, or dermatomyositis.

In some embodiments, the method comprises a test, wherein said test comprises a) administering the test aliquot of the composition to said subject; b) inducing, e.g., by application of electrical stimulation, e.g., percutaneous stimulation or surface stimulation, a test muscle contraction, e.g., the flexor hallucis brevis muscle; and c) evaluating the effect of administering the test aliquot of the composition on test muscle contraction, e.g., by evaluating the electrical activity of said test muscle, e.g., by EMG. In some embodiments, wherein step a is performed before step b. In some embodiments, wherein step a is performed after step b.

In another aspect, the present disclosure features a solid oral dosage form comprising a single gingerol analog and less than about 25% of the total gingerol concentration in the solid oral dosage form (e.g., by mole or by weight) of a related analog thereof (e.g., less than about 20%, less than about 15%, less than about 10%, less than about 5%, less than about 3%, less than about 1%, less than about 0.1%, or is substantially free of a related analog thereof). In an embodiment, the solid oral dosage form comprises a single gingerol analog and is substantially free of a related analog thereof.

In some embodiments, the single gingerol analog is, e.g., 6-gingerol. In some embodiments, the amount of the single gingerol analog (e.g., 6-gingerol) in the solid oral dosage form is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single gingerol analog (e.g., 6-gingerol) in the solid oral dosage form is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single gingerol analog (e.g. 6-gingerol) in the solid oral dosage form is from about 0.0001% (w/v) or (w/w) to about 10% (w/v) or (w/w). In some embodiments, the amount of the single gingerol analog (e.g. 6-gingerol) in the solid dosage form is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In some embodiments, the solid oral dosage form further comprises a single shogaol analog, e.g., 6-shogaol. In some embodiments, the amount of the single shogaol analog (e.g., 6-shogaol) in the solid oral dosage form is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single shogaol analog (e.g., 6-shogaol) in the solid oral dosage form is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single shogaol analog (e.g. 6-shogaol) in the solid oral dosage form is from about 0.0001% (w/v) or (w/w) to about 10% (w/v) or (w/w). In some embodiments, the amount of the single shogaol analog (e.g. 6-shogaol) in the solid oral dosage form is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In some embodiments, the molar ratio of the single gingerol analog (e.g., 6-gingerol) and the single shogaol analog (e.g., 6-shogaol) is from about 1000:1 to about 1:1 (e.g., about 750:1, about 500:1, about 250:1, about 200:1, about 100:1, about 50:1, about 25:1, about 10:1, about 5:1, about 2.5:1).

In another aspect, the present disclosure features a solid oral dosage form comprising a single shogaol analog and less than about 25% of the total shogaol concentration in the solid oral dosage form (e.g., by mole or by weight) of a related analog thereof (e.g., less than about 20%, less than about 15%, less than about 10%, less than about 5%, less than about 3%, less than about 1%, less than about 0.1%, or is substantially free of a related analog thereof). In an embodiment, the solid oral dosage form comprises a single shogaol analog and is substantially free of a related analog thereof.

In some embodiments, the single shogaol analog is, e.g., 6-shogaol. In some embodiments, the amount of the single shogaol analog (e.g., 6-shogaol) in the solid oral dosage form is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single shogaol analog (e.g., 6-shogaol) in the solid oral dosage form is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single shogaol analog (e.g. 6-shogaol) in the solid oral dosage form is from about 0.0001% (w/v) or (w/w) to about 10% (w/v) or (w/w). In some embodiments, the amount of the single shogaol analog (e.g. 6-shogaol) in the solid oral dosage form is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In some embodiments, the solid oral dosage form further comprises a single gingerol analog, e.g., 6-gingerol. In some embodiments, the amount of the single gingerol analog (e.g., 6-gingerol) in the solid oral dosage form is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single gingerol analog (e.g., 6-shogaol) in the solid oral dosage form is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single gingerol analog (e.g. 6-gingerol) in the solid oral dosage form is from about 0.0001% (w/v) or (w/w) to about 10% (w/v) or (w/w). In some embodiments, the amount of the single gingerol analog (e.g. 6-gingerol) in the solid oral dosage form is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single gingerol analog (e.g., 6-gingerol) and the single shogaol analog (e.g., 6-shogaol) is from about 1000:1 to about 1:1 (e.g., about 750:1, about 500:1, about 250:1, about 200:1, about 100:1, about 50:1, about 25:1, about 10:1, about 5:1, about 2.5:1).

In any and all aspects, in some embodiments, the solid oral dosage form further comprises a single capsaicinoid analog, e.g., capsaicin. In some embodiments, the amount of the single capsaicinoid analog (e.g., capsaicin) in the solid oral dosage form is from 0.001 mg to about 20 mg (e.g., about 0.01 mg to about 10 mg, about 0.05 mg to about 5 mg, about 0.075 mg to about 2.5 mg, about 0.1 mg to about 1 mg). In some embodiments, the amount of the single capsaicinoid analog (e.g. capsaicin) in the solid oral dosage form is from about 0.0001% (w/v) or (w/w) to about 10% (w/v) or (w/w). In some embodiments, the amount of the single capsaicinoid analog (e.g. capsaicin) in the solid oral dosage form is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single gingerol analog (e.g., 6-gingerol) and the single capsaicinoid analog (e.g., capsaicin) is from about 1000:1 to about 5:1 (e.g., about 750:1, about 500:1, about 250:1, about 200:1, about 100:1, about 50:1, about 25:1, about 10:1). In some embodiments, the molar ratio of the single shogaol analog (e.g., 6-shogaol) and the single capsaicinoid analog (e.g., capsaicin) is from about 1000:1 to about 5:1 (e.g., about 750:1, about 500:1, about 250:1, about 200:1, about 100:1, about 50:1, about 25:1, about 10:1).

In some embodiments, the solid oral dosage form further comprises trans-cinnamaldehyde. In some embodiments, the amount of the trans-cinnamaldehyde in the solid oral dosage form is from 5 mg to about 1000 mg (e.g., about 10 mg to about 250 mg, about 15 mg to about 200 mg, about 20 mg to about 100 mg, about 25 mg to about 75 mg). In some embodiments, the amount of trans-cinnamaldehyde in the solid oral dosage form is from about 0.0001% (w/v) or (w/w) to about 20% (w/v) or (w/w). In some embodiments, the amount of trans-cinnamaldehyde in the composition is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single gingerol analog (e.g., 6-gingerol) and trans-cinnamaldehyde is from about 1000:1 to about 1:1 (e.g., about 750:1, about 500:1, about 250:1, about 200:1, about 100:1, about 50:1, about 25:1, about 10:1, about 5:1, about 2.5:1). In some embodiments, the molar ratio of the single shogaol analog (e.g., 6-shogaol) and trans-cinnamaldehyde is from about 1000:1 to about 1:1 (e.g., about 750:1, about 500:1, about 250:1, about 200:1, about 100:1, about 50:1, about 25:1, about 10:1, about 5:1, about 2.5:1).

In some embodiments, the solid oral dosage form has a residence time of greater than about 5 seconds in the mouth of a subject, e.g., greater than about 6 seconds, about 7 seconds, about 8 seconds, about 9 seconds, about 10 seconds, about 11 seconds, about 12 seconds, about 13 seconds, about 14 seconds, about 15 seconds, about 20 seconds, about 25 seconds, about 30 seconds, about 45 seconds, about 60 seconds, about 90 seconds, about 2 minutes, about 3 minutes, about 4 minutes, about 5 minutes, or more. In some embodiments, the solid oral dosage form has a residence time of greater than about 60 seconds in the mouth of a subject, e.g., greater than about 90 seconds, about 2 minutes, about 3 minutes, about 4 minutes, about 5 minutes, about 6 minutes, about 7 minutes, about 8 minutes, about 9 minutes, about 10 minutes, or more.

In some embodiments, the solid oral dosage form is formulated to have minimal systemic exposure in the subject, e.g., wherein the systemic exposure is measured through the concentration of the active ingredient (e.g., a gingerol, e.g., 6-gingerol) in the blood, urine, or tissue (e.g., adipose tissue) of the subject. In some embodiments, the solid oral dosage form is formulated to have less than about 50% total systemic exposure in a subject, e.g., less than about 40%, less than about 30%, less than about 20%, less than about 10%, less than about 5%, less than about 1%, less than about 0.5%, less than about 0.1%, less than about 0.05%, less than about 0.01%, or less systemic exposure in a subject.

In another aspect, the present disclosure features a solid oral dosage form comprising a single capsaicinoid analog and less than about 25% of the total capsaicinoid concentration in the solid oral dosage form (e.g., by mole or by weight) of a related analog thereof (e.g., less than about 20%, less than about 15%, less than about 10%, less than about 5%, less than about 3%, less than about 1%, less than about 0.1%, or is substantially free of a related analog thereof). In an embodiment, the solid oral dosage form comprises a single capsaicinoid analog and is substantially free of a related analog thereof.

In some embodiments, the single capsaicinoid analog is, e.g., capsaicin. In some embodiments, the amount of the single capsaicinoid analog (e.g., capsaicin) in the solid oral dosage form is from 0.001 mg to about 20 mg (e.g., about 0.01 mg to about 10 mg, about 0.05 mg to about 5 mg, about 0.075 mg to about 2.5 mg, about 0.1 mg to about 1 mg). In some embodiments, the amount of the single capsaicinoid analog (e.g. capsaicin) in the solid oral dosage form is from about 0.0001% (w/v) or (w/w) to about 10% (w/v) or (w/w). In some embodiments, the amount of the single capsaicinoid analog (e.g. capsaicin) in the solid oral dosage form is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In some embodiments, the solid oral dosage form further comprises a single gingerol analog, e.g., 6-gingerol. In some embodiments, the amount of the single gingerol analog (e.g., 6-gingerol) in the solid dosage form is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single gingerol analog (e.g., 6-gingerol) in the solid oral dosage form is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single gingerol analog (e.g. 6-gingerol) in the solid oral dosage form is from about 0.0001% (w/v) or (w/w) to about 10% (w/v) or (w/w). In some embodiments, the amount of the single gingerol analog (e.g. 6-gingerol) in the solid oral dosage form is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single capsaicinoid analog (e.g., capsaicin) and the single gingerol analog (e.g., 6-gingerol) is from about 1000:1 to about 1:1 (e.g., about 750:1, about 500:1, about 250:1, about 200:1, about 100:1, about 50:1, about 25:1, about 10:1, about 5:1, about 2.5:1).

In some embodiments, the solid oral dosage form further comprises a single shogaol analog, e.g., 6-shogaol. In some embodiments, the amount of the single shogaol analog (e.g., 6-shogaol) in the solid dosage form is from about 0.01 mg to about 1000 mg (e.g., about 0.1 mg to about 500 mg, about 0.5 mg to about 250 mg, about 10 mg to about 100 mg, about 25 mg to about 75 mg). In some embodiments, the amount of the single shogaol analog (e.g., 6-shogaol) in the solid oral dosage form is from about 5 mg to about 1000 mg (e.g., about 10 mg to about 250 mg, about 15 mg to about 200 mg, about 20 mg to about 100 mg, about 25 mg to about 75 mg). In some embodiments, the amount of the single shogaol analog (e.g. 6-shogaol) in the composition is from about 0.0001% (w/v) or (w/w) to about 10% (w/v) or (w/w). In some embodiments, the amount of the single shogaol analog (e.g. 6-shogaol) in the solid oral dosage form is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single capsaicinoid analog (e.g., capsaicin) and the single shogaol analog (e.g., 6-shogaol) is from about 1000:1 to about 1:1 (e.g., about 750:1, about 500:1, about 250:1, about 200:1, about 100:1, about 50:1, about 25:1, about 10:1, about 5:1, about 2.5:1).

In some embodiments, the solid oral dosage form further comprises trans-cinnamaldehyde. In some embodiments, the amount of the trans-cinnamaldehyde in the solid oral dosage form is from 5 mg to about 1000 mg (e.g., about 10 mg to about 250 mg, about 15 mg to about 200 mg, about 20 mg to about 100 mg, about 25 mg to about 75 mg). In some embodiments, the amount of trans-cinnamaldehyde in the solid oral dosage form is from about 0.0001% (w/v) or (w/w) to about 20% (w/v) or (w/w). In some embodiments, the amount of trans-cinnamaldehyde in the solid oral dosage form is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the molar ratio of the single capsaicinoid analog (e.g., capsaicin) and trans-cinnamaldehyde is from about 1000:1 to about 1:1 (e.g., about 750:1, about 500:1, about 250:1, about 200:1, about 100:1, about 50:1, about 25:1, about 10:1, about 5:1, about 2.5:1).

In some embodiments, the solid oral dosage form has a residence time of greater than about 5 seconds in the mouth of a subject, e.g., greater than about 6 seconds, about 7 seconds, about 8 seconds, about 9 seconds, about 10 seconds, about 11 seconds, about 12 seconds, about 13 seconds, about 14 seconds, about 15 seconds, about 20 seconds, about 25 seconds, about 30 seconds, about 45 seconds, about 60 seconds, about 90 seconds, about 2 minutes, about 3 minutes, about 4 minutes, about 5 minutes, or more. In some embodiments, the solid oral dosage form has a residence time of greater than about 60 seconds in the mouth of a subject, e.g., greater than about 90 seconds, about 2 minutes, about 3 minutes, about 4 minutes, about 5 minutes, about 6 minutes, about 7 minutes, about 8 minutes, about 9 minutes, about 10 minutes, or more.

In some embodiments, the solid oral dosage form is formulated to have minimal systemic exposure in the subject, e.g., wherein the systemic exposure is measured through the concentration of the active ingredient (e.g., a capsaicinoid, e.g., capsaicin) in the blood, urine, or tissue (e.g., adipose tissue) of the subject. In some embodiments, the solid oral dosage form is formulated to have less than about 50% total systemic exposure in a subject, e.g., less than about 40%, less than about 30%, less than about 20%, less than about 10%, less than about 5%, less than about 1%, less than about 0.5%, less than about 0.1%, less than about 0.05%, less than about 0.01%, or less systemic exposure in a subject.

In another aspect, the present disclosure features a solid oral dosage form comprising a single capsaicinoid analog and one of a single gingerol analog or single shogaol analog, wherein the composition is substantially free of a related analog thereof.

In another aspect, the present disclosure features a tablet (e.g., an orally disintegrating tablet) comprising a single gingerol analog, wherein the composition is substantially free of a related analog thereof.

In another aspect, the present disclosure features a tablet (e.g., an orally disintegrating tablet) comprising a single shogaol analog, wherein the composition is substantially free of a related analog thereof.

In another aspect, the present disclosure features a tablet (e.g., an orally disintegrating tablet) comprising a single capsaicinoid analog, wherein the composition is substantially free of a related analog thereof.

In another aspect, the present disclosure features a tablet (e.g., an orally disintegrating tablet) comprising a single capsaicinoid analog and one of a single gingerol analog or single shogaol analog, wherein the composition is substantially free of a related analog thereof.

In another aspect, the present disclosure features a lozenge or fast melt formulation comprising a single gingerol analog, wherein the composition is substantially free of a related analog thereof.

In another aspect, the present disclosure features a lozenge or fast melt formulation comprising a single shogaol analog, wherein the composition is substantially free of a related analog thereof.

In another aspect, the present disclosure features a lozenge or fast melt formulation comprising a single capsaicinoid analog, wherein the composition is substantially free of a related analog thereof.

In another aspect, the present disclosure features a lozenge or fast melt formulation comprising a single capsaicinoid analog and one of a single gingerol analog or single shogaol analog, wherein the composition is substantially free of a related analog thereof.

In another aspect, the present disclosure features a beverage comprising a single gingerol analog, wherein the composition is substantially free of a related analog thereof.

In another aspect, the present disclosure features a beverage comprising a single shogaol analog, wherein the composition is substantially free of a related analog thereof.

In another aspect, the present disclosure features a beverage comprising a single capsaicinoid analog, wherein the composition is substantially free of a related analog thereof.

In another aspect, the present disclosure features a beverage comprising a single capsaicinoid analog and one of a single gingerol analog or single shogaol analog, wherein the composition is substantially free of a related analog thereof.

In another aspect, the present disclosure features a product formulated for oral administration to a subject comprising a single gingerol analog, a single shogaol analog, or a single capsaicinoid analog that is substantially free of related analogs thereof, wherein if present, the amount of the single gingerol analog or single shogaol analog in the product is between about 0.01 mg and about 1000 mg; and if present, the amount of the single capsaicinoid analog in the product is between about 0.001 mg and about 100 mg.

In another aspect, the present disclosure features a product formulated for oral administration to a subject comprising a single gingerol analog, a single shogaol analog, or a single capsaicinoid analog that is substantially free of related analogs thereof, wherein if present, the amount of the single gingerol analog or single shogaol analog in the product is between about 0.05 mM and about 500 mM; and if present, the amount of the single capsaicinoid analog in the product is between about 0.05 μM and about 100 μM.

In another aspect, the present disclosure features a product formulated for oral administration to a subject comprising a single gingerol analog, a single shogaol analog, or a single capsaicinoid analog that is substantially free of related analogs thereof, wherein if present, the amount of the single gingerol analog or single shogaol analog in the product is between about 0.0001% (w/v) or (w/w) and about 10% (w/v) or (w/w) or between about 0.001% (w/v) or (w/w) and about 30% (w/v) or (w/w) or between about 0.01% (w/v) or (w/w) and about 50% (w/v) or (w/w); and if present, the amount of the single cap saicinoid analog in the product is between about 0.0001% (w/v) or (w/w) and about 5% (w/v) or (w/w) or between about 0.01% (w/v) or (w/w) and about50% (w/v) or (w/w).

In another aspect, the present disclosure features a pharmaceutical composition formulated for oral administration, wherein the composition comprises an effective amount of a single gingerol analog, a single shogaol analog, or a single capsaicinoid analog and is substantially free of related analogs thereof, and a pharmaceutically acceptable excipient, wherein if present, the dosage of the single gingerol analog or single shogaol analog in the product is between about 0.01 mg and about 1000 mg; and if present, the dosage of the single capsaicinoid analog in the product is between about 0.001 mg and about 100 mg.

In another aspect, the present disclosure features a pharmaceutical composition formulated for oral administration, wherein the composition comprises an effective amount of a single capsaicinoid analog and one of a single gingerol or a single shogaol analog and is substantially free of related analogs thereof, and a pharmaceutically acceptable excipient, wherein the dosage of the single gingerol analog or single shogaol analog in the product is between about 0.01 mg and about 1000 mg; and the dosage of the single capsaicinoid analog in the product is between about 0.001 mg and about 100 mg.

In another aspect, the present disclosure features a pharmaceutical composition formulated for oral administration, wherein the composition comprises an effective amount of a single gingerol analog, a single shogaol analog, or a single capsaicinoid analog and is substantially free of related analogs thereof, and a pharmaceutically acceptable excipient, wherein if present, the dosage of the single gingerol analog or single shogaol analog in the product is between about 0.05 mM and about 500 mM; and if present, the dosage of the single capsaicinoid analog in the product is between about 0.05 μM and about 100 μM.

In another aspect, the present disclosure features a pharmaceutical composition formulated for oral administration, wherein the composition comprises an effective amount of a single capsaicinoid analog and one of a single gingerol analog or a single shogaol analog and is substantially free of related analogs thereof, and a pharmaceutically acceptable excipient, wherein the dosage of the single gingerol analog or single shogaol analog in the product is between about 0.05 mM and about 500 mM; and the dosage of the single capsaicinoid analog in the product is between about 0.05 μM and about 100 μM.

In another aspect, the present disclosure features a pharmaceutical composition formulated for oral administration, wherein the composition comprises an effective amount of a single gingerol analog, a single shogaol analog, or a single capsaicinoid analog and is substantially free of related analogs thereof, and a pharmaceutically acceptable excipient, wherein if present, the dosage of the single gingerol analog or single shogaol analog in the product is between about 0.0001% (w/v) or (w/w) and about 10% (w/v) or (w/w) or between about 0.001% (w/v) or (w/w) and about 30% (w/v) or (w/w) or between about 0.01% (w/v) or (w/w) and about 50% (w/v) or (w/w); and if present, the dosage of the single capsaicinoid analog in the product is between about 0.0001% (w/v) or (w/w) and about 5% (w/v) or (w/w) or between about 0.01% (w/v) or (w/w) and about 50% (w/v) or (w/w).

In another aspect, the present disclosure features a pharmaceutical composition formulated for oral administration, wherein the composition comprises an effective amount of a single capsaicinoid analog and one of a single gingerol analog or a single shogaol analog and is substantially free of related analogs thereof, and a pharmaceutically acceptable excipient, wherein the dosage of the single gingerol analog or single shogaol analog in the product is between about 0.0001% (w/v) or (w/w) and about 10% (w/v) or (w/w) or between about 0.001% (w/v) or (w/w) and about 30% (w/v) or (w/w) or between about 0.01% (w/v) or (w/w) and about 50% (w/v) or (w/w); and the dosage of the single capsaicinoid analog in the product is between about 0.0001% (w/v) or (w/w) and about 5% (w/v) or (w/w) or between about 0.01% (w/v) or (w/w) and about 50% (w/v) or (w/w).

In any of the above-mentioned aspects, in some embodiments, the solid dosage forms, lozenges, fast melt formulations, beverages, tablets, products, or compositions (e.g., pharmaceutical compositions) may comprise any of the features described below. In some embodiments, said solid dosage form, lozenge, fast melt formulation, beverage, tablet, product, or composition (e.g., a pharmaceutical composition) comprises a single gingerol analog (e.g., 6-gingerol). In some embodiments, the amount of the gingerol (e.g., 6-gingerol) in said solid dosage form, lozenge, fast melt formulation, beverage, tablet, product, or composition (e.g., a pharmaceutical composition) is from about 0.01 mg to about 1000 mg (e.g., about 0.5 to about 500 mg, about 10 mg to about 250 mg, about 15 mg to about 200 mg, about 20 mg to about 100 mg, about 25 mg to about 75 mg). In some embodiments, the amount of the gingerol (e.g. 6-gingerol) in the solid dosage form, lozenge, fast melt formulation, beverage, tablet, product, or composition (e.g., a pharmaceutical composition) is from about 0.0001% (w/v) or (w/w) to about 10% (w/v) or (w/w). In some embodiments, the amount of the gingerol (e.g., 6-gingerol) in the solid dosage form, lozenge, fast melt formulation, beverage, tablet, product, or composition (e.g., a pharmaceutical composition) is from about 0.001% (w/v) or (w/w) to about 30% (w/v) or (w/w). In some embodiments, the amount of the single gingerol analog (e.g. 6-gingerol) in the solid dosage form, lozenge, fast melt formulation, beverage, tablet, product, or composition (e.g., a pharmaceutical composition) is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In some embodiments, the solid dosage form, lozenge, fast melt formulation, beverage, tablet, product, or composition (e.g., a pharmaceutical composition) comprises a single shogaol analog (e.g., 6-shogaol). In some embodiments, the amount of the shogaol (e.g., 6-shogaol) in the solid dosage form, lozenge, fast melt formulation, beverage, tablet, product, or composition (e.g., a pharmaceutical composition) is from about 0.01 mg to about 1000 mg (e.g., about 0.5 to about 500 mg, about 10 mg to about 250 mg, about 15 mg to about 200 mg, about 20 mg to about 100 mg, about 25 mg to about 75 mg). In some embodiments, the amount of the shogaol (e.g., 6-shogaol) in the solid dosage form, lozenge, fast melt formulation, beverage, tablet, product, or composition (e.g., a pharmaceutical composition) is from about 0.0001% (w/v) or (w/w) to about 10% (w/v) or (w/w). In some embodiments, the amount of the shogaol (e.g., 6-shogaol) in the solid dosage form, lozenge, fast melt formulation, beverage, tablet, product, or composition (e.g., a pharmaceutical composition) is from about 0.001% (w/v) or (w/w) to about 30% (w/v) or (w/w). In some embodiments, the amount of the single shogaol analog (e.g. 6-shogaol) in the solid dosage form, lozenge, fast melt formulation, beverage, tablet, product, or composition (e.g., a pharmaceutical composition) is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In some embodiments, the solid dosage form, lozenge, fast melt formulation, beverage, tablet, product, or composition (e.g., a pharmaceutical composition) comprises a single capsaicinoid analog (e.g., capsaicin). In some embodiments, the amount of the capsaicinoid (e.g., capsaicin) in the solid dosage form, lozenge, fast melt formulation, beverage, tablet, product, or composition (e.g., a pharmaceutical composition) is from about 0.001 mg to about 20 mg (e.g., about 0.01 mg to about 10 mg, about 0.05 mg to about 5 mg, about 0.075 mg to about 2.5 mg, about 0.1 mg to about 1 mg). In some embodiments, the amount of the capsaicinoid (e.g., capsaicin) in the solid dosage form, lozenge, fast melt formulation, beverage, tablet, product, or composition (e.g., a pharmaceutical composition) is from about 0.01 mg to about 50 mg. In some embodiments, the amount of the capsaicinoid (e.g., capsaicin) in the solid dosage form, lozenge, fast melt formulation, beverage, tablet, product, or composition (e.g., a pharmaceutical composition) is from about 0.0001% (w/v) or (w/w) to about 10% (w/w) or (w/w). In some embodiments, the amount of the single capsaicinoid analog (e.g. capsaicin) in the solid dosage form, lozenge, fast melt formulation, beverage, tablet, product, or composition (e.g., a pharmaceutical composition) is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). In some embodiments, the solid dosage form, lozenge, fast melt formulation, beverage, tablet, product, or composition (e.g., a pharmaceutical composition) comprises a single gingerol analog (e.g., 6-gingerol) and a single capsaicinoid analog (e.g., capsaicin). In some embodiments, the molar ratio of the gingerol (e.g., 6-gingerol) and the capsaicinoid (e.g., capsaicin) is from about 1000:1 to about 5:1 (e.g., about 750:1, about 500:1, about 250:1, about 200:1, about 100:1, about 50:1, about 25:1, about 10:1).

In some embodiments, the solid dosage form, lozenge, fast melt formulation, beverage, tablet, product, or composition (e.g., a pharmaceutical composition) comprises a single shogaol analog (e.g., 6-shogaol) and a single capsaicinoid analog (e.g., capsaicin). In some embodiments, the molar ratio of the shogaol (e.g., 6-shogaol) and the capsaicinoid (e.g., capsaicin) is from about 1000:1 to about 5:1 (e.g., about 750:1, about 500:1, about 250:1, about 200:1, about 100:1, about 50:1, about 25:1, about 10:1).

In an embodiment, the solid dosage form, lozenge, fast melt formulation, beverage, tablet, product, or composition (e.g., a pharmaceutical composition) comprises a single gingerol analog (e.g., 6-gingerol), a single shogaol analog (e.g., 6-shogaol), or a single capsaicinoid analog (e.g., capsaicin), and is substantially free of related analogs thereof. In some embodiments, the solid dosage form, lozenge, fast melt formulation, beverage, tablet, product, or composition (e.g., a pharmaceutical composition) comprises a single gingerol analog (e.g., 6-gingerol), a single shogaol analog (e.g., 6-shogaol), or a single capsaicinoid analog (e.g., capsaicin), and less than about 25% of the total gingerol, shogaol, or capsaicinoid concentration in the composition (e.g., by mole or by weight) of related analogs thereof (e.g., less than about 20%, less than about 15%, less than about 10%, less than about 5%, less than about 3%, less than about 1%, less than about 0.1%, or is substantially free of a related analog thereof).

In some embodiments, the solid dosage form, lozenge, fast melt formulation, beverage, tablet, product, or composition (e.g., a pharmaceutical composition) further comprises trans-cinnamaldehyde. In some embodiments, the amount of trans-cinnamaldehyde in the solid dosage form, lozenge, fast melt formulation, beverage, tablet, product, or composition (e.g., a pharmaceutical composition) is from about 5 mg to about 1000 mg (e.g., about 10 mg to about 750 mg, about 25 mg to about 500 mg, about 50 mg to about 250 mg, about 100 mg to about 200 mg). In some embodiments, the amount of trans-cinnamaldehyde in the solid dosage form, lozenge, fast melt formulation, beverage, tablet, product, or composition (e.g., a pharmaceutical composition) is from about 0.0001% (w/v) or (w/w) to about 10% (w/v). In some embodiments, the amount of trans-cinnamaldehyde in the solid dosage form, lozenge, fast melt formulation, beverage, tablet, product, or composition (e.g., a pharmaceutical composition) is from about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w).

In some embodiments, the solid dosage form, lozenge, fast melt formulation, beverage, tablet, product, or composition (e.g., a pharmaceutical composition) further comprises an additional component (e.g., a pharmaceutically acceptable excipient or formulation base). In some embodiments, the pharmaceutically acceptable excipient comprises carboxymethylcellulose sodium, citric acid, glycerin, sodium citrate. In some embodiments, the formulation base comprises water, ethanol, or phosphate buffered saline. In some embodiments, the pharmaceutically acceptable salt comprises mannitol, lactose, sucrose, magnesium stearate, magnesium aluminometasilicate or silica (e.g., fumed silica).

In some embodiments, the solid dosage form, lozenge, fast melt formulation, beverage, tablet, product, or composition (e.g., a pharmaceutical composition) further comprises an oil, a lipophilic additive, a flavoring agent, a coloring agent, a dissolving agent, a viscosity modifier, an electrolyte, a vitamin, a mineral, an antioxidant, or a preservative. In some embodiments, the oil is selected from the group consisting of: vegetable oil, mineral oil, soya oil, sunflower oil, corn oil, olive oil, nut oil, and liquid paraffin. In some embodiments, the lipophilic additive is selected from the group consisting of: polyethylene glycol, polymethacrylate, fatty acid mono-, di-, or triglycerides, palmitic acid, stearic acid, behenic acid, polyethylene glycol fatty acid esters, candelilla wax, carnauba wax, polyethylene oxide wax, and petroleum wax. In some embodiments, the flavoring agent is a sweetening agent. In some embodiments, the sweetening agent comprises cane sugar, pectin, karo syrup (e.g., light karo syrup, dark karo syrup), honey, saccharin (e.g., sodium saccharin), or sorbitol.

In some embodiments, the product or composition (e.g., a pharmaceutical composition) is formulated as a liquid or a solid dosage form. In some embodiments, the product or composition (e.g., a pharmaceutical composition) is formulated as a liquid dosage form. In some embodiments, the liquid dosage form is selected from the group consisting from an emulsion, a microemulsion, a solution, a suspension, a syrup, a linctus, a drop, a spray, a mouthwash, and an elixir. In some embodiments, the composition is formulated as a beverage.

In some embodiments, the product or composition (e.g., a pharmaceutical composition) is formulated as a solid dosage form. In some embodiments, the solid dosage form is selected from the group consisting of a tablet, a capsule, a powder, a crystal, a paste, a gel, a lozenge, a troche, a gum, a candy, a chew, a foodstuff, a dissolving strip, a film, and a semi-solid formulation. In some embodiments, the capsule is a hard or soft capsule. In some embodiments, the tablet is an orally disintegrating tablet, sublingual tablet, a chewable tablet, or buccal tablet.

In some embodiments, the product or composition (e.g., a pharmaceutical composition) is formulated as orally disintegrating tablet, a sublingual tablet, or buccal tablet and comprises an excipient.

In some embodiments, the pH of the solid dosage form, lozenge, fast melt formulation, beverage, tablet, product, or composition (e.g., a pharmaceutical composition) is between about 1.5 and about 7.5, e.g, between about 2.0 and about 5.0, or between about 2.5 and about 4.0.

In some embodiments, the solid dosage form, lozenge, fast melt formulation, beverage, tablet, product, or composition (e.g., a pharmaceutical composition) has a residence time of greater than about 5 seconds in the mouth of a subject, e.g., greater than about 6 seconds, about 7 seconds, about 8 seconds, about 9 seconds, about 10 seconds, about 11 seconds, about 12 seconds, about 13 seconds, about 14 seconds, about 15 seconds, about 20 seconds, about 25 seconds, about 30 seconds, about 45 seconds, about 60 seconds, about 90 seconds, about 2 minutes, about 3 minutes, about 4 minutes, about 5 minutes, or more. In some embodiments, the solid dosage form, lozenge, fast melt formulation, beverage, tablet, product, or composition (e.g., a pharmaceutical composition) has a residence time of greater than about 60 seconds in the mouth of a subject, e.g., greater than about 90 seconds, about 2 minutes, about 3 minutes, about 4 minutes, about 5 minutes, about 6 minutes, about 7 minutes, about 8 minutes, about 9 minutes, about 10 minutes, or more.

### BRIEF DESCRIPTION OF THE DRAWINGS

FIG. 1 is a graph depicting the co-activation of TRPV1 and TRPA1 ion channels in a sub-population of human dorsal root ganglion neurons as described in Example 1.

FIG. 2A is a picture of stimulating electrodes placed on the flexor brevis muscle on the upper arch of the foot in the region of the medial plantar nerve as described in Example 2. FIG. 2B is a picture of stimulating electrodes placed on the flexor brevis muscle below the lateral malleolus of the ankle as described in Example 2. FIGS. 2C-2E are picture of EMG recording electrodes placed over the belly of the flexor halluces brevis muscle. FIG. 2C is a picture of the EMG recording electrodes placed over the belly of the flexor hallucis brevis muscle, with a first reference electrode at the medial malleolus of the ankle as described in Example 2. FIG. 2D is a picture of the EMG recording electrodes placed over the belly of the flexor hallucis brevis muscle, with a second reference electrode at the base of the flexor hallucis brevis muscle as described in Example 2. FIG. 2E is a picture of the EMG recording electrodes placed over the belly of the flexor hallucis brevis muscle, with a third reference electrode toward the big toe on the widest region of the flexor hallucis brevis muscle as described in Example 2.

FIG. 3 is a graph summarizing the average change in AUC values for all subjects administered Treatments A-H compared by administered treatment over the entire monitored period for each time point. Each subject was monitored prior to treatment, as well as both 1 and 2 hours following treatment.

FIG. 4 is a graph summarizing the average change in AUC values for all subjects administered Treatments A-H compared by administered treatment over 140 seconds of the monitoring period.

FIG. 5 is a graph summarizing the average change in AUC values for all subjects administered Treatments I-P compared by administered treatment over the entire monitored period for each time point. Each subject was monitored prior to treatment, as well as both 1 and 2 hours following treatment.

FIG. 6 is a graph summarizing the average change in AUC values for all subjects administered Treatments I-P compared by administered treatment over 140 seconds of the monitoring period.

FIG. 7 is an HPLC chromatogram of the ginger extract used to prepare Treatment A. Each peak was identified by peak retention time comparisons to compounds known to be present in the parental extract.

FIG. 8 is a table summarizing the EC₅₀ values of Treatment K and Treatment L for the TRPA1 and TRPV1 ion channels.

FIG. 9 is a table summarizing the ratio of AUC values relative to baseline of Treatment K, Treatment L, Treatment M, and Treatment N compared with the extract mixture of Treatment A and vehicle.

FIG. 10 is a chart showing the efficacy of exemplary compositions of the invention to reduce cramp intensity cramp intensity as described in Example 3.

FIG. 11 is a graph depicting the average delta AUCF values obtained for each treatment of an exemplary composition of the present invention as outlined in Example 4. FIG. 12 is a graph depicting the average ratio AUCF values obtained for each treatment of an exemplary composition of the present invention as outlined in Example 4.

FIG. 13 is a graph depicting the average ratio AUCF values obtained for each treatment of an exemplary composition of the present invention over time as outlined in Example 4. FIG. 14 is a graph depicting the average delta AUCF values obtained for each treatment of an exemplary composition of the present invention over time as outlined in Example 4. FIG. 15 is a graph depicting the average delta AUCF values obtained for each treatment of an exemplary composition of the present invention over time as outlined in Example 4.

FIG. 16 is a graph showing the inhibition of electrically-induced cramps in normal healthy subjects as dependent on dosage amount of an exemplary composition of the present invention as outlined in Example 4.

### DETAILED DESCRIPTION OF THE INVENTION

Unwanted or abnormal muscle contractions are uncontrollable contractions of one or more muscles, and may be triggered by movement or exercise or may occur spontaneously. While unwanted or abnormal muscle contractions may arise in any muscle in the body, they occur most frequently in the muscles of the foot, calf, thigh (e.g., quadriceps or hamstring), hands, arms (e.g., bicep or tricep), abdomen, and muscles along the rib cage. The methods, products, and compositions of the present invention are directed to the prevention and treatment of unwanted or abnormal muscle contractions, e.g., muscle cramps, spasms, dystonias, fasciculations, and the like. In some embodiments, the methods, products, and compositions of the present invention comprise an agonist or activator of a TRP channel (e.g., TRPV1 or TRPA1) or an ASIC channel, or a use thereof. In some embodiments, the methods, products, and compositions of the present invention comprise a gingerol (e.g., 6-gingerol), a shogaol (e.g., 6-shogaol), a capsaicinoid (e.g., capsaicin) or a use thereof, and are substantially free of a related analog thereof.

### Definitions

The use of the words “a” or “an” when used in conjunction with the term “comprising” herein may mean “one,” but are also consistent with the meaning of “one or more,” “at least one,” and “one or more than one.”

The term “acidulant” as used herein refers to an acidic compound (e.g., an acid) used to lower the pH of a composition. In some embodiments, the pH can be lowered in the range of about 2.5 to about 6.5 (e.g., pH of 2.5, 3.0, 3.5, 4.0, 4.5, 5.0, 5.5, 6.0, or 6.5). In some embodiments, the pH can be lowered in the range of about 1.5 to about 5.0 (e.g., pH of 1.5, 2.0, 2.5, 3.0, 3.5, 4.0, 4.5, 5.0), for example, in the range of about 1.5 to about 4.4 (e.g., pH of 1.5, 2.0, 2.5, 3.0, 3.5, 4.0, 4.4).

“Acquire” or “acquiring” as the terms are used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., applying or measuring a current to or from a subject, or capturing a signal from a subject or sample or performing a synthetic or analytical method) to obtain the value or physical entity. “Indirectly acquiring” refers to receiving the value or physical entity from another party or source (e.g., a third party laboratory that directly acquired the physical entity or value). Directly acquiring a value or physical entity includes performing a process that includes a physical change in a physical substance or the use of a machine or device. Exemplary changes include applying a current to, or measuring a current from, the muscle of a subject. Directly acquiring a value includes performing a process that uses a machine or device, e.g., a device to induce a cramp or a device to measure a parameter related to a cramp.

The term “agonist,” as used herein, refers to a molecule that stimulates a biological response. In some embodiments, an agonist is an activator. In some embodiments, the agonists and activators referred to herein may activate a TRP ion channel (e.g., TRPV1 or TRPA1) or an ASIC ion channel.

The term “administering” and “administration” refers to a mode of delivery. A daily dosage can be divided into one, two, three or more doses in a suitable form to be administered one, two, three or more times throughout a time period. In preferred embodiments of the present invention, compositions and solutions are administered orally.

The terms “analog” or “related analogs” as used herein in regard to a compound or compounds refer to a substance that has a similar chemical structure to another compound, but differs from it with respect to a certain component or components.

The term “derivative” as used herein refers to a substance produced from another substance either directly or by modification or partial substitution.

“Dystonia” as used herein refers to sustained muscle contractions that cause repetitive movements or twisting and other abnormal postures. In some embodiments, a dystonia can occur in a limb, e.g., a hand or foot.

“Fasciculation” as used herein refers to a series of small, rapid involuntary muscle contractions and relaxations. In some embodiments, fasciculations are commonly known as “muscle twitches”.

“Muscle cramp” as used herein is a muscle cramp which is treated with the composition described herein. In some embodiments, it is not induced but rather arises spontaneously either from activity or underlying disease etiology, e.g., athletic activity or a night cramp. In some embodiments, a muscle cramp is induced for test purposes. In some embodiments, the muscle cramp comprises a cramp in a muscle other than the muscle of the test muscle cramp. In some embodiments, the muscle cramp can be a contraction of a skeletal muscle or the smooth muscle. In some embodiments, the muscle cramp is a contraction of a skeletal muscle, e.g., the flexor hallucis brevis muscle.

“Muscle spasm” as used herein refers to an involuntary contraction or a muscle, or even a few fibers of a muscle. In some embodiments, the magnitude or duration of a spasm is less than that of a cramp.

As used herein, the terms “prevent” or “preventing” as used in the context of a disorder or disease, refer to administration of an agent to a subject such that the onset of at least one symptom of the disorder or disease is delayed as compared to what would be seen in the absence of administration of said agent. As compared with an equivalent untreated control, such prevention is at least 5%, 10%, 20%, 30%, 40%, 50%, 60%, 70%, 80%, 90%, 95%, 99%, or 100%, as measured by any standard technique.

The term “spasticity” as referred to herein is the uncontrolled tightening or contracting of the muscles that is common in individuals with spinal cord injuries and a variety of nervous system diseases. In some embodiments, spasticity refers to a velocity-dependent increase in the tonic stretch reflex (muscle tone) with exaggerated tendon jerks, clonus, and spasms, resulting from the hyper excitability of the stretch reflex.

The term “subject” as used herein refers to a mammal, including, but not limited to, a human or non-human mammal, such as a bovine, equine, canine, ovine, or feline mammal. In some embodiments, the term subject refers to a human (e.g., a human male or female).

As used herein, the term “substantially pure” refers to a composition that is free of organic and/or inorganic species that do not activate the TRPV1, TRPA1, or/or ASIC channels, and where 60%, 65%, 70%, 75%, 80%, 85%, 90%, 95%, 97%, 98%, 99%, or 99.5% (w/w) of the composition is comprised of a single chemical species. Substantially pure compositions can be prepared and analyzed using standard methods known in the art (e.g., chromatographic separation, extractions, and the like). In some embodiments, substantially pure compositions do not include isomeric impurities (e.g., geometric isomers) and/or salts or solvates of a particular chemical species.

A “test muscle contraction” as used herein is a muscle contraction, typically induced, e.g., by the application of electrical current, in the subject. Stimulation can be applied to induce a muscle contraction that recapitulates a naturally-occurring muscle cramp, muscle spasm, dystonia, or fasciculation, e.g., a test muscle cramp, a test muscle spasm, a test muscle dystonia, or a test muscle fasciculation. In embodiments, the test muscle cramp comprises a cramp in the flexor hallucis brevis muscle. In some embodiments, efficacy in inducing a test muscle cramp in a subject is indicative of efficacy in treating muscle cramp, e.g., with a composition described herein. In other embodiments, efficacy in treating the test muscle cramp is indicative of efficacy in treating muscle cramp, spasticity, dystonias, or fasciculations.

“Treat” or “treating” as used herein refers to administering a composition for therapeutic purposes or administering treatment to a subject already suffering from a disorder to improve the subject's condition. By “treating a condition or disorder” or “alleviating a condition or disorder” is meant that the condition or disorder (e.g., an unwanted or abnormal muscle contraction) and the symptoms associated with the condition or disorder are, e.g., prevented, alleviated, reduced, cured, or placed in a state of remission. As compared with an equivalent untreated control, such alleviation or degree of treatment is at least 5%, 10%, 20%, 30%, 40%, 50%, 60%, 70%, 80%, 90%, 95%, 99%, or 100%, as measured by any standard technique.

The term “viscosity” as used herein refers to a measurement of a fluid's internal resistance to flow (e.g., “thickness”). Viscosity is generally expressed in centipoise (cP) or pascal-seconds.

Other features and advantages of the invention will be apparent from the Detailed Description, Examples, and Claims.

### Products and Compositions

The products and compositions described herein are comestible formulations suitable for administration to a subject (e.g., a human) and include one or more agents capable of preventing or treating an unwanted or abnormal muscle contraction (e.g., muscle cramps, spasms, spasticity, dystonias, fasciculations). In some embodiments, the products and compositions described herein comprise a compound capable of activating an ion channel (e.g., a TRP or ASIC ion channel). In some embodiments, the products and compositions described herein comprise a gingerol (e.g., 6-gingerol), a shogaol (e.g., 6-shogaol), or a capsaicinoid (e.g., capsaicin). In some embodiments, the products and compositions further comprise an excipient as described herein. In some embodiments, the compositions described herein are pharmaceutical compositions. Exemplary, non-limiting compositions include those that are solid dosage forms for oral administration (e.g., tablets, capsules, powders, crystals, pastes, gels, lozenges (e.g., liquid filled lozenges), troches, gums, candies, chews, foodstuffs, films, and the like), liquid dosage forms for oral administration (e.g., emulsions, microemulsions, solutions, suspensions, syrups (e.g., syrup concentrates), linctuses, drops, sprays, elixirs, mouthwashes, and the like), ready-to-drink beverages, dry compositions that can be reconstituted with a liquid (e.g., powders, granules, or tablets that may be reconstituted with water), gels, semi-solids (e.g., ice cream, pudding, or yogurt), frozen liquids (e.g., ice pops), hard candies, dissolving strips (e.g., an edible strip containing pullulan and compositions of the invention), and chewing gums.

### TRP Channels and ASIC Channels

In some embodiments, the products and compositions described herein comprise a compound capable of activating an ion channel (e.g., a TRP or ASIC ion channel, e.g., a gingerol (e.g., 6-gingerol), a shogaol (e.g., 6-shogaol), a capsaicinoid (e.g., capsaicin)). Transient Receptor Protein (TRP) channels are a family of nonselective ion channels that function as cellular sensors and respond to and integrate diverse signals, including temperature, mechanical stress, exogenous chemicals, and endogenous chemicals, such as intracellular and extracellular messengers. These channels are involved in multiple functions, including pain, temperature, and mechanical sensation, calcium and magnesium homeostasis, lysosomal function, cardiovascular regulation, and control of cell growth and proliferation. Misregulation or aberrant function of TRP channels have also been implicated in numerous muscle conditions (Brinkmeier, H. Adv Exp Med Biol (2011) 704:749-758; Yang X. R. et al. Adv Exp Med Biol (2010) 661:109-122; Szallasi, A. (Ed.) TRP Channels as Therapeutic Targets (2015) Waltham, Mass.: Elsevier). Members of the TRP channel family share some structural similarity and are organized in sub-families, comprising TRPA, TRPC, TRPV, TRPM, TRPML, TRMPN, and TRPP. Each of these sub-families comprise subunit genes, which include, for example, TRPV1, TRPV2, TRPV4, TRPV3, TRPV5, TRPV6, TRPA1, TRPP3, TRPP2, TRPP5, TRPC4, TRPC5, TRPC1, TRPC3, TRPC7, TRPC6, TRPM1, TRPM3, TRPM6, TRPM7, TRPM4, TRPM5, TRPM2, TRPM8, TRPML1, TRPML3, and TRPML2. The compositions described herein may comprise at least one activator or agonist of any of the TRP channels, and may be used to prevent or treat unwanted or abnormal muscle contraction (e.g., muscle cramps, spasms, dystonias, fasciculations).

Acid-sensing ion channels (ASIC) are neuronal voltage-insensitive cationic channels that are activated by extracellular protons. ASIC channels are primarily expressed in the nervous system, and conduct mostly Nat Recent evidence has linked certain muscle conditions and disorders to aberrant ASIC channel function (see, e.g., Gautam, M. and Benson, C. J. (2013) FASEB J 27:793-802). There are four ASIC channel genes, ASIC1, ASIC2, ASIC3 and ASIC4, which encode at least six ASIC channels, ASIC3, ASCI4 and splice variants of ASIC1, and ASIC2, ASIC1a, ASIC1b, ASIC2a, ASIC2b. The compositions described herein may comprise at least one activator or agonist of any of the ASIC channels and may be used in prevention or treatment of unwanted or abnormal muscle contraction (e.g., muscle cramps, spasms, dystonias, fasciculations).

### TRP and ASIC Channel Activators

In some embodiments, the products and compositions described in the present invention feature a compound capable of activating an ion channel (e.g., a TRP or ASIC ion channel). Exemplary compounds include, but are not limited to, gingerols (e.g., 6-gingerol, 8-gingerol, 10-gingerol, 12-gingerol, 14-gingerol, and other synthetic and naturally occurring variants thereof, e.g., methoxy-10-gingerol), shogaols (e.g., 6-shogaol, 8-shogaol, 10-shogaol, 12-shogaol, 14-shogaol, and other synthetic and naturally occurring variants thereof), paradols (e.g., 6-paradol), gingerdiones (e.g., 10-gingerdione), gingerenone A, capsaicinoids (e.g., capsaicin and dihydrocapsaicin), and cinnamaldehyde (e.g., trans-cinnamaldehyde) In some embodiments the products and compositions described herein comprise gingerols (e.g., 6-gingerol, 8-gingerol, 10-gingerol, 12-gingerol, 14-gingerol, and other synthetic and naturally occurring variants thereof, e.g., methoxy-10-gingerol), shogaols (e.g., 6-shogaol, 8-shogaol, 10-shogaol, 12-shogaol, 14-shogaol, and other synthetic and naturally occurring variants thereof), capsaicinoids (e.g., capsaicin and dihydrocapsaicin), paradols (e.g., 6-paradol), and cinnamaldehyde (e.g., trans-cinnamaldehyde). in some embodiments, the products and compositions described herein comprise a single gingerol analog (e.g., 6-gingerol), a single shogaol analog (e.g., 6-shogaol), a single capsaicinoid analog (e.g., capsaicin), a single paradol (e.g., 6-paradol) or cinnamaldehyde (e.g., trans-cinnamaldehyde), and is substantially free of an analog thereof. In some embodiments, the products and compositions described herein comprise 6-gingerol, 6-shogaol, capsaicin, 6-paradol, or cinnamaldehyde. In some embodiments, the products and compositions described herein comprise 6-gingerol, 6-shogaol, capsaicin, 6-paradol or cinnamaldehyde, and are substantially free of a related analog thereof. In some embodiments, the products and compositions described herein comprise an isotopically labeled analog of a compound described herein, e.g., a deuterated analog of a gingerol (e.g., 6-gingerol), a shogaol (e.g., 6-shogaol), a capsaicinoid (e.g., capsaicin), a paradol (e.g., 6-paradol) or cinnamaldehyde (e.g., trans-cinnamaldehyde).

A gingerol (e.g., 6-gingerol), shogaol (e.g., 6-shogaol), capsaicinoid (e.g., capsaicin), paradol (e.g., 6-paradol) or cinnamaldehyde (e.g., trans-cinnamaldehyde) may be present in a composition of the invention at a concentration range of about 0.001% to about 10% by weight by weight (w/w) based on the total weight of the composition (e.g., about 0.001, about 0.005, about 0.01, about 0.1, about 0.5, about 1, about 2, about 3, about 4, about 5, about 6, about 7, about 8, about 9, or about 10%) or at a concentration range of about 0.001% to about 10% by weight by volume (w/v) based on the total volume of the composition (e.g., about 0.001, about 0.005, about 0.01, about 0.1, about 0.5, about 1, about 2, about 3, about 4, about 5, about 6, about 7, about 8, about 9, or about 10%), though one or more of these compounds may be present in lower or higher concentrations (e.g., less than about 0.01%, e.g., about 0.008%, about 0.005%, about 0.004%, about 0.001% (w/w) or (w/v), or more than about 10%, e.g., about 12%, about 15%, about 20%, about 30%, about 35%, about 40%, about 50% (w/w) or (w/v)). In some embodiments, a gingerol (e.g., 6-gingerol), shogaol (e.g., 6-shogaol), capsaicinoid (e.g., capsaicin), paradol (e.g., 6-paradol) or cinnamaldehyde (e.g., trans-cinnamaldehyde) may be present in a composition of the invention at a concentration range of about 0.001% (w/v) or (w/w) to about 50% (w/v) or (w/w), or about 0.01% (w/v) or (w/w) to about 50% (w/v) or (w/w), or about 0.1% (w/v) or (w/w) to about 50% (w/v) or (w/w), or about 1% (w/v) or (w/w) to about 50% (w/v) or (w/w), or about 5% (w/v) or (w/w) to about 50% (w/v) or (w/w). A gingerol (e.g., 6-gingerol), shogaol (e.g., 6-shogaol), capsaicinoid (e.g., capsaicin), a paradol (e.g., 6-paradol) or cinnamaldehyde (e.g., trans-cinnamaldehyde) may be present at a concentration range of about 0.001 mg to about 1000 mg per unit dosage (e.g., about 0.001 mg, about 0.005 mg, about 0.01 mg, about 0.05 mg, about 0.1 mg, about 0.5 mg, about 0.75 mg, about 1 mg, about 5 mg, about 10 mg, about 15 mg, about 20 mg, about 25 mg, about 30 mg, about 35 mg, about 40 mg, about 45 mg, about 50 mg, about 60 mg, about 75 mg, about 100 mg, about 150 mg, about 200 mg, about 250 mg, about 300 mg, about 350 mg, about 400 mg, about 500 mg, about 600 mg, about 700 mg, about 800 mg, or about 900 mg).

Each of these components is described in more detail below.

### Gingerols, Shogaols, and Parasols

Ginger, derived from the rhizomes Zingiber oficinale Roscoe, is a well-known spice and has been a central component of foods for thousands of years. It is most frequently prescribed as a traditional medicine for treatment of gastrointestinal and respiratory diseases, and has been shown to be effective in promoting blood circulation and for the removal of blood stasis (Koo, K. L. et al. Thromb Res (2001) 103:387-397; Shih. H. C. et al. Int J Mol Sci (2014) 15:3926-3951). Ginger rhizomes produce hundreds of compounds that have been classified into several groups, e.g., zingerone, gingerols, gingerdiols, gingerdiones, paradols, and shogaols, in addition to other closely related analogs. The largest class of compounds present in ginger extract is the gingerols, of which 6-gingerol is the most abundant. However, the closely related species 8-gingerol, 10-gingerol, and 12-gingerol are also present. In some embodiments, the products and compositions described herein comprise a single gingerol analog (e.g., 6-gingerol) and are substantially free of an analog thereof.

In some embodiments, the products and compositions described herein comprise a single gingerol analog (e.g., 6-gingerol) with a purity of greater than about 90%, e.g., relative to other synthetically generated impurities. In some embodiments, the purity of the single gingerol analog (e.g., 6-gingerol) is greater than about 90%, e.g., about 91%, about 92%, about 93%, about 94%, about 95%, about 96%, about 97%, about 98%, about 99%, about 99.9%, or more, e.g., relative to other synthetically generated impurities. In some embodiments, the purity of the single gingerol analog (e.g., 6-gingerol) is greater than about 95%, e.g., about 96%, about 97%, about 98%, about 99%, about 99.9% or more, e.g., relative to other synthetically generated impurities. In some embodiments, the purity of the single gingerol analog (e.g., 6-gingerol) is greater than about 98%, e.g., about 98.5%, about 99%, about 99.5%, about 99.9%, about 99.99%, or more, e.g., relative to other synthetically generated impurities.

In some embodiments, the products and compositions described herein comprise a mixture of two stereoisomers of 6-gingerol, e.g., (R)-6-gingerol and (S)-6-gingerol. In some embodiments, the mixture comprises a ratio of (R)-6-gingerol and (S)-6-gingerol of about 1:1 (e.g., a racemic mixture). In some embodiments, the mixture comprises a ratio of (R)-6-gingerol to (S)-6-gingerol of about 51:49, about 52:48, about 53:47, about 54:46, about 55:45, about 60:40, about 65:35, about 70:30, about 75:25, about 80:20, about 85:15, about 90:10, about 95:5, about 99:1, or higher. In some embodiments, the mixture comprises a ratio of (R)-6-gingerol to (S)-6-gingerol of about 70:30, about 75:25, about 80:20, about 85:15, about 90:10, about 95:5, about 99:1, or higher. In some embodiments, the mixture comprises a ratio of (R)-6-gingerol to (S)-6-gingeroll of about 80:20, about 85:15, about 90:10, about 95:5, about 99:1, or higher.

In some embodiments, the products and compositions described herein comprise (R)-6-gingerol and less than about 50% of (S)-6-gingerol, e.g., less than about 45%, less than about 40%, less than about 35%, less than about 30%, less than about 25%, less than about 20%, less than about 15%, less than about 10%, less than about 5%, less than about 2.5%, less than about 1%, less than about 0.5%, less than about 0.1%, or less than about 0.05% of (S)-6-gingerol. In some embodiments, the products and compositions described herein comprise (R)-6-gingerol and are substantially free of (S)-6-gingerol.

Shogaols are another class of compounds produced by ginger rhizomes, with the species 6-shogaol being the most abundant. However, the most common method for production of shogaols is through the dehydration of gingerols during drying and heating of ginger extract. Paradols are also naturally found in ginger extract, although these molecules (e.g., 6-paradol) are produced in larger amounts by other plants, including the Guinea pepper (Aframomum nielegueta). Both shogaols and paradols, like gingerols, have been shown to be potent activators of various ion channels, e.g., the TRPV1 channel and the TRPA1 channel (see, e.g., Riera, C. E. et al. Br J Pharmacol (2009) 157:1398-1409), as well as exhibiting antimicrobial and antitumor activity (see, e.g., Chung, W. Y. et al. Mutat Res (2001) 496:199-206; Jolad, S. D. et al. Phytochemistry (2005) 66:1614-1635).

In some embodiments, the products and compositions described herein comprise a single shogaol analog (e.g., 6-shogaol) with a purity of greater than about 90%, e.g., relative to other synthetically generated impurities. In some embodiments, the purity of the single shogaol analog (e.g., 6-shogaol) is greater than about 90%, e.g., about 91%, about 92%, about 93%, about 94%, about 95%, about 96%, about 97%, about 98%, about 99%, about 99.9%, or more, e.g., relative to other synthetically generated impurities. In some embodiments, the purity of the single shogaol analog (e.g., 6-shogaol) is greater than about 95%, e.g., about 96%, about 97%, about 98%, about 99%, about 99.9% or more, e.g., relative to other synthetically generated impurities. In some embodiments, the purity of the single shogaol analog (e.g., 6-shogaol) is greater than about 98%, e.g., about 98.5%, about 99%, about 99.5%, about 99.9%, about 99.99%, or more, e.g., relative to other synthetically generated impurities.

In some embodiments, the products and compositions described herein comprise a single paradol analog (e.g., 6-paradol) with a purity of greater than about 90%, e.g., relative to other synthetically generated impurities. In some embodiments, the purity of the single paradol analog (e.g., 6-paradol) is greater than about 90%, e.g., about 91%, about 92%, about 93%, about 94%, about 95%, about 96%, about 97%, about 98%, about 99%, about 99.9%, or more, e.g., relative to other synthetically generated impurities. In some embodiments, the purity of the single paradol analog (e.g., 6-paradol) is greater than about 95%, e.g., about 96%, about 97%, about 98%, about 99%, about 99.9% or more, e.g., relative to other synthetically generated impurities. In some embodiments, the purity of the single paradol analog (e.g., 6-paradol) is greater than about 98%, e.g., about 98.5%, about 99%, about 99.5%, about 99.9%, about 99.99%, or more, e.g., relative to other synthetically generated impurities.

### Capsaicin

Capsaicin and several related compounds belong to a class of compounds called capsaicinoids and are produced as secondary metabolites by chili peppers. Capsaicin and other capsaicinoids are odorless, fat soluble compounds with a highly pungent taste, and are responsible for providing the spicy flavor of chili peppers through direct activation of TRP ion channels. Capsaicin has been shown to exert multiple pharmacological and physiological effects including analgesia and anticancer, anti-inflammation, antioxidant, and anti-obesity activities (Hayman, M. and Kam, P. C. A. Curr Anaesth Crit Care (2008) 19:338-343).

In some embodiments, the products and compositions described herein comprise a single capsaicinoid analog (e.g., capsaicin) with a purity of greater than about 90%, e.g., relative to other synthetically generated impurities. In some embodiments, the purity of the single capsaicinoid analog (e.g., capsaicin) is greater than about 90%, e.g., about 91%, about 92%, about 93%, about 94%, about 95%, about 96%, about 97%, about 98%, about 99%, about 99.9%, or more, e.g., relative to other synthetically generated impurities. In some embodiments, the purity of the single capsaicinoid analog (e.g., capsaicin) is greater than about 95%, e.g., about 96%, about 97%, about 98%, about 99%, about 99.9% or more, e.g., relative to other synthetically generated impurities. In some embodiments, the purity of the single capsaicinoid analog (e.g., capsaicin) is greater than about 98%, e.g., about 98.5%, about 99%, about 99.5%, about 99.9%, about 99.99%, or more, e.g., relative to other synthetically generated impurities.

### Trans-Cinnamaldehyde

Trans-cinnamaldehyde is one of the major components of cinnamon, a spice obtained from the inner bark of trees in the Cinnatnomum family, and is responsible for its characteristic flavor and odor. Trans-cinnamaldehyde is widely used as a commercial food additive and scent. In addition, trans-cinnamaldehyde has been shown to have potent anti-inflammatory and antioxidant activity, e.g, through its ability to inhibit production of nitric oxide and suppress of the transcription factor NF-κB (Cássia da Silveira e Sá et al. Molecules (2014) 19:1459-1480).

In some embodiments, the products and compositions described herein comprise trans-cinnamaldehyde with a purity of greater than about 90%, e.g., relative to other synthetically generated impurities. In some embodiments, the purity of the trans-cinnamaldehyde is greater than about 90%, e.g., about 91%, about 92%, about 93%, about 94%, about 95%, about 96%, about 97%, about 98%, about 99%, about 99.9%, or more, e.g., relative to other synthetically generated impurities. In some embodiments, the purity of the trans-cinnamaldehyde is greater than about 95%, e.g., about 96%, about 97%, about 98%, about 99%, about 99.9% or more, e.g., relative to other synthetically generated impurities. In some embodiments, the purity of the trans-cinnamaldehyde is greater than about 98%, e.g., about 98.5%, about 99%, about 99.5%, about 99.9%, about 99.99%, or more, e.g., relative to other synthetically generated impurities.

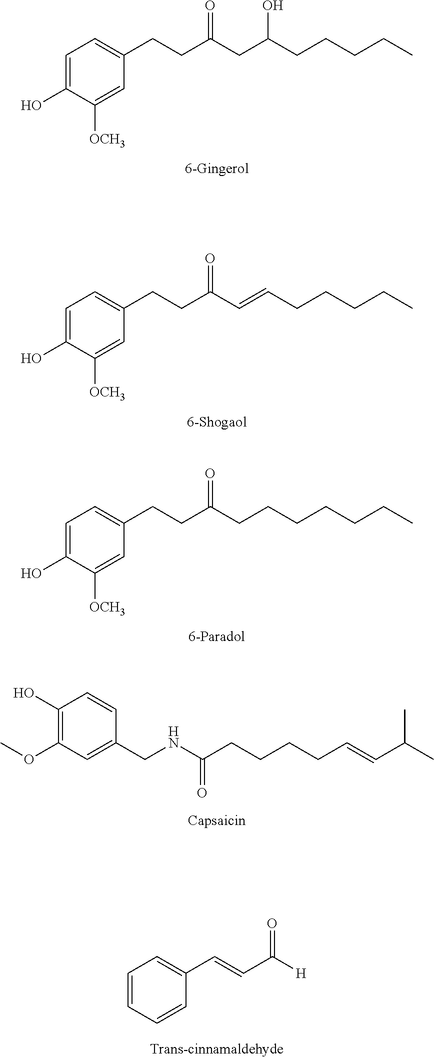

### Additional Components of the Composition

The composition of the present invention may additionally include, for example, electrolytes (e.g., potassium salt or other salts), buffering agents, sweeteners, flavoring and coloring agents, vitamins, minerals, preservatives, viscosity modifiers, thickening agents, dissolving agents, solvents, and antioxidants as described below. Other exemplary excipients are described in Handbook of Pharmaceutical Excipients, 7^{th} Edition, Rowe et al., Eds., Pharmaceutical Press (2009).

### Viscosity Modifiers and Thickening Agents

Viscosity is the ratio of shear stress to shear rate, expressed as dynes-second/cm², or poise. A centipoise (cP) is one one-hundredth of a poise.

The composition of the present invention may have a viscosity greater than water (i.e., about 1.0 cP at 20° C.), e.g., about 100, 200, 300, 400, 500, 1000, 1500, 2000, 2500, 3000, 3500, 4000, 4500, 5000, 6000, 7000, 8000, 9000 cP or more. If a consistency of corn syrup is desired, viscosities in the range of about 2500 cP are suitable. If a consistency of a soft gel or honey is desired, viscosities in the range of about 10000 cP to about 15000 cP are suitable. For pudding-like products, viscosities in the range of about 30000 cP to about 38000 cP are desirable. Viscosity of the compositions of the present invention may be measured with, e.g., a rheometer or viscometer, though additional methods of measuring viscosity are known in the art.

Viscosity modifiers and thickening agents may be added to compositions of the present invention. Such viscosity modifiers and thickening agents include, for example, collagen, gellan gum, carbohydrate gel-forming polymers, carob bean gum, locust bean gum, carrageenan, alginates (e.g., alginic acid, sodium alginate, potassium alginate, ammonium alginate, and calcium alginate), agar, guar gum, xanthan gum, microcrystalline cellulose, carboxymethyl cellulose, ethyl cellulose, clear starch, pectin, gelatin, arrowroot, cornstarch, katakuri starch, potato starch, sago, tapioca, furcellaran, karo syrup (e.g., light karo syrup and dark karo syrup), glycerin, and sodium pyrophosphate. A viscosity modifier or thickening agent may be present in the composition in an amount of from about 0.01% to about 10% by weight based on the total weight or volume of the composition (e.g., about 0.01, about 0.1, about 0.5, about 1, about 2%, about 3%, about 4%, about 5%, about 6%, about 7%, about 8%, about 9%, or about 10%), though the viscosity modifier or thickening agent may be present in lower or higher concentrations (e.g., about 10%, about 20%, about 30%, about 40%, about 50%, about 60%, about 70%, about 80%, or about 90%). In some embodiments, the viscosity modifier or thickening agent is present in the composition from about 40% to about 60% (e.g, about 50%).

### Electrolytes and Buffering Agents

Exemplary electrolytes and buffering agents include potassium salts, chloride salts, bromide salts, sodium salts, magnesium salts, calcium salts, citrate salts, acetate salts, phosphate salts, salicylates, bicarbonate salts (e.g., sodium bicarbonate), lactate salts, sulphate salts, tartrate salts, benzoate salts, selenite salts, molybdate salts, iodide salts, oxides, and combinations thereof. An electrolyte or buffering agent may be present in a composition of the invention at a concentration range of about 0.01% to about 10% by weight based on the total weight or volume of the composition (e.g., about 0.01%, about 0.02%, about 0.03%, about 0.04%, about 0.05%, about 0.1%, about 0.5%, about 1%, about 2%, about 3%, about 4%, about 5%, about 6%, about 7%, about 8%, about 9%, or about 10%), though an electrolyte or buffering agent may be present in lower or higher concentrations.

In certain embodiments, the compositions of the present invention include high concentrations of potassium (e.g., potassium chloride). The concentration of potassium in the composition may be, e.g., about 0.01%, about 0.02%, about 0.03%, about 0.04%, about 0.05%, about 0.1%, about 0.5%, about 1%, about 2%, about 3%, about 4%, about 5%, about 6%, or about 7% or more by weight based on the total weight or volume of the composition.

In certain embodiments, the compositions of the present invention include high concentrations of magnesium (e.g., magnesium chloride). The concentration of magnesium in the composition may be, e.g., about 0.01%, about 0.02%, about 0.03%, about 0.04%, about 0.05%, about 0.1%, about 0.5%, about 1%, about 2%, about 3%, about 4%, about 5%, about 6%, or about 7% or more by weight based on the total weight or volume of the composition.

In some embodiments, an electrolyte or buffering agent may be added to the compositions of the present invention to affect the pH level. In some embodiments, the pH of the composition, e.g., with the addition of an electrolyte or buffering agent, is e.g., about 1.5, about 2.0, about 2.5, about 3.0, about 3.5, about 4.0, about 4.5, about 5.0, about 5.5, about 6.0, about 6.5, about 7.0, about 7.5, about 8.0, or about 8.5.

### Sweeteners

Sweeteners may be included in the compositions of the invention. Exemplary sweeteners include corn syrup (e.g., high fructose corn syrup or karo syrup), mannose, maltose, glucose polymers, sucrose (e.g., cane sugar or beet sugar), glucose, dextrose, lactose, galactose, fructose, polysaccharides (e.g., malodextrins), rice syrup, honey, and natural fruit juices (e.g., orange juice, papaya juice, pineapple juice, apple juice, grape juice, apricot juice, pear juice, tomato juice, agave nectar, or cranberry juice). Additionally, non- or low-caloric sweeteners can be used in the compositions of the invention. Examples of such non-caloric or low-caloric sweeteners include, but are not limited to, saccharin, sodium saccharin, cyclamates, acetosulfam, sorbitol, mannitol, sucralose, xylitol, erythritol, Stevia extract, L-aspartyl-L-phenyl-alanine ester (e.g., aspartame), L-aspartyl-D-alanine alkyl amides, L-aspartyl-L-1-hydroxymethylalkaneamide, and L-aspartyl-1-hydroxyethylalkaneamide. In some embodiments, sweeteners may be present in a composition of the invention at a concentration range of about 2% to about 20% by weight based on the total weight or volume of the composition (e.g., about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%), though sweeteners may be present in lower or higher concentrations.

### Flavoring and Coloring Agents

Exemplary flavors and flavoring agents include almond oil, amaretto oil, anethole, anise oil, apple, benzaldehyde, blackberry, black walnut oil, blueberry, caraway, caraway oil, cardamom oil, cardamom seed, cherry juice, cherry syrup, chocolate, cinnamon, cinnamon oil, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, mint (e.g., peppermint, spearmint), myristica oil, orange oil, orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint oil, strawberry, sucrose, thyme oil, tolu balsam, vanilla, vanillin, watermelon, and wild cherry syrup. Additional flavoring agents may be found in Food Chemicals Codex and Fenaroli's Handbook of Flavor Ingredients. Flavoring agents may be present in a composition of the invention at a concentration range of about 0.01% to about 20% by weight based on the total weight or volume of the composition (e.g., about 0.05%, about 0.1%, about 0.5%, about 1%, about 2%, about 3%, about 4%, about 5%, about 7.5%, about 10%, about 12.5%, about 15%, about 17.5%, or about 20%), though flavoring agents may be present in lower or higher concentrations.

Small amounts of a coloring agent may be utilized in the compositions of the present invention. Coloring agents include, e.g., beta-carotene, riboflavin dyes, FD&C dyes (e.g., Yellow No. 5, Blue No. 1, Blue No. 2, and Red No. 40), FD&C lakes, chlorophylls and chlorophyllins, caramel coloring, annatto, cochineal, turmeric, saffron, paprika, and fruit, vegetable, and/or plant extracts (e.g., grape, black currant, aronia, carrot, beetroot, red cabbage, elderberry, and hibiscus extracts). The amount of coloring agent used will vary depending on the agents used in the composition and the color intensity desired in the finished product. Coloring agents may be present in a composition of the invention at a concentration range of about 0.01% to about 20% by weight based on the total weight or volume of the composition (e.g., about 0.05%, about 0.1%, about 0.5%, about 1%, about 2%, about 3%, about 4%, about 5%, about 7.5%, about 10%, about 12.5%, about 15%, about 17.5%, or about 20%), though coloring agents may be present in lower or higher concentrations.

### Vitamins and Minerals

Non-limiting examples of vitamins and minerals that may be included in the compositions of the present invention include, e.g., choline bitartate, niacinamide, thiamin, folic acid, d-calcium pantothenate, biotin, vitamin A, vitamin C, vitamin B₁ hydrochloride, vitamin B₂, vitamin B₃, vitamin B₆ hydrochloride, vitamin B₁₂, vitamin D, vitamin E acetate, vitamin K, and salts of calcium, potassium, magnesium, zinc, iodine, iron, and copper. In some embodiments, vitamins and minerals may be present in a composition of the invention at a concentration range of about 0.01% to about 50% by weight based on the total weight or volume of the composition (e.g., about 0.05%, about 0.1%, about 0.5%, about 1%, about 2%, about 3%, about 4%, about 5%, about 7.5%, about 10%, about 12.5%, about 15%, about 17.5%, about 20%, about 25%, about 30%, about 35%, about 40%, about 45%, or about 50%), though vitamins and minerals may be present in lower or higher concentrations. In some embodiments, when included in a composition of the invention, the composition may contain at least about 5%, about 10%, about 15%, about 20%, about 25%, about 30%, about 35%, about 40%, about 45%, or about 50% of the U.S. recommended daily intake (RDI) for such vitamins and minerals.

### Preservatives

A preservative may additionally be utilized in the compositions described herein. Exemplary preservatives include, for example, sorbate, polysorbate (e.g., polysorbate 20, polysorbate 40, polysorbate 80), a paraben (e.g., methylparaben sodium, propylparaben sodium), benzoate, and polyphosphate preservatives (e.g., sorbic acid, benzoic acid, calcium sorbate, sodium sorbate, potassium sorbate, calcium benzoate, sodium benzoate, potassium benzoate, and mixtures thereof). In some embodiments, the preservative may be present in a composition of the invention at a concentration range of about 0.0005% to about 0.5% (e.g., about 0.0005%, about 0.001%, about 0.005%, about 0.01%, about 0.05%, about 0.1%, or about 0.5%) by weight based on the total weight or volume of the composition, though preservatives may be present in lower or higher concentrations.

In some embodiments, a preservative may be added to the compositions of the present invention to affect the pH. In some embodiments, the pH of the composition, e.g., with the addition of a preservative, is e.g., about 1.5, about 2.0, about 2.5, about 3.0, about 3.5, about 4.0, about 4.5, about 5.0, about 5.5, about 6.0, about 6.5, about 7.0, about 7.5, about 8.0, or about 8.5. In some embodiments, the pH of the composition, e.g., with the addition of a preservative, is within the range of about 1.5 to about 7.5, about 1.75 to about 7.0, about 2.0 to about 6.5, about 2.1 to about 6.0, about 2.2 to about 5.5, about 2.3 to about 5.0, about 2.4 to about 4.5, about 2.5 to about 4.0, about 2.6 to about 3.5. In some embodiments, the pH of the composition, e.g., with the addition of a preservative, is within the range of about 1.5 to about 4.0.

### Antioxidants

An antioxidant agent may also be included in the compositions to, for example, reduce exercise-induced oxidative stress. Exemplary antioxidants include vitamin C and vitamin E; beta-carotene, lutein, or other carotenoids; cyanidin, delphinidin, malvidin, or other anthocyanidins; apigenin, luteolin, or other flavones; hesperitin, naringenin, or other flavonones; isorhamnetin, quercetin, kaempferol or other flavonols; butylated hydroxyanisole and butylated hydroxytoluene; and epigallocatechin-3-gallate, epicatechin, thearubigins, or other flavan-3-ols. In some embodiments, an antioxidant may be present in a composition of the invention at a concentration range of about 0.0005% to about 0.5% (e.g., about 0.0005%, about 0.001%, about 0.005%, about 0.01%, about 0.05%, about 0.1%, or about 0.5%) by weight based on the total weight or volume of the composition, though antioxidants may be present in lower or higher concentrations.

### Dissolving Agent or Solvent

A dissolving agent or solvent may also be included in the compositions to, for example, improve the suspension or emulsification of particular components. In addition, certain dissolving agents or solvents may have a preservative function. Exemplary dissolving agents or solvents include acetic acid, acetone, butanol, dimethyl sulfoxide, ethanol, ethyl acetate, isopropanol, methanol, petroleum ether and the like. In some embodiments, a dissolving agent or solvent may be present in a composition of the invention at a concentration range of about 0.0005% to about 0.5% (e.g., about 0.0005%, about 0.001%, about 0.005%, about 0.01%, about 0.05%, about 0.1%, or about 0.5%) by weight based on the total weight or volume of the composition, though dissolving agents or solvents may be present in lower or higher concentrations.

Additional components of the compositions described herein may include amino acids (e.g., leucine, isoleucine, lysine, methionine, phenylalanine, threonine, tryptophan, and valine), stimulants (e.g., caffeine), emulsifying agents, carbon dioxide (e.g., to carbonate a liquid composition), stabilizers, humectants, anticaking agents, or herbal extracts. These components may be included at levels from about 0.0005% to about 25% (e.g., about 0.0005%, about 0.001%, about 0.005%, about 0.01%, about 0.05%, about 0.1%, about 0.5%, about 1%, about 5%, about 10%, about 15%, about 20%, or about 25%) by weight based on the total volume of the composition, though an additional component may be present in lower or higher concentrations.

### Formulations and Methods of Preparing Compositions

The compositions and solutions of the present invention may be formulated as ready-to-drink beverages, concentrates (e.g., syrups), dry compositions (e.g., powders, granules, or tablets that may be reconstituted with a liquid (e.g., with water), gels, solids, semi-solids (e.g., ice cream, pudding, or yogurt), frozen liquids (e.g., ice pops), lozenges or hard candies, mouthwashes, dissolving strips (e.g., an edible strip containing pullulan and compositions of the invention), and chewing gum. Formulation of these compositions may require the use of a formulation base, which is a substance or material mixed with or added to the ion channel activator and pharmaceutically acceptable excipient in order to achieve the desired form.

In solid dosage forms for oral administration (e.g., tablets (e.g., orally disintegrating tablets, sublingual tablets, or buccal tablets), capsules, powders, crystals, pastes, gels, lozenges (e.g., liquid filled lozenges), troches, gums, candies, chews, foodstuffs, dissolving strips, films, semi-solid formulations, dragées, and the like), the compositions of the invention are mixed with a pharmaceutically-acceptable carrier, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, hydroxypropyl cellulose, hydroxypropyl methylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, crospovidine, croscarmellose (e.g., croscarmellose sodium), hypromellose, sodium starch glycolate, certain silicates, and sodium carbonate; (5) solution retarding agents, such as paraffin; (6) absorption accelerators, such as quaternary ammonium compounds; (7) wetting agents, such as, for example, acetyl alcohol, sodium lauryl sulfate, and glycerol monostearate; (8) absorbents, such as kaolin, colloidal silicon dioxide, and bentonite clay; (9) lubricants, such a talc, calcium stearate, magnesium stearate, solid polyethylene glycols, sodium lauryl sulfate, sodium stearyl fumurate, stearic acid, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

In some embodiments, compositions of the present invention are formulated to increase residence time in the upper gastrointestinal tract (e.g., mouth or esophagus). In some embodiments, the composition is formulated to have an extended residence time in the upper gastrointestinal tract of a subject (e.g., a residence time of greater than about 5 seconds in the mouth of a subject, e.g., a residence time of greater than about 6 seconds, about 7 seconds, about 8 seconds, about 9 seconds, about 10 seconds, about 11 seconds, about 12 seconds, about 13 seconds, about 14 seconds, about 15 seconds, about 20 seconds, about 25 seconds, about 30 seconds, about 45 seconds, about 60 seconds, about 90 seconds, about 2 minutes, about 3 minutes, about 4 minutes, about 5 minutes, or more in the mouth of a subject). In some embodiments, the composition is formulated to have an extended residence time in the upper gastrointestinal tract of a subject (e.g., a residence time of greater than about 60 seconds in the mouth of a subject, e.g., greater than about 90 seconds, about 2 minutes, about 3 minutes, about 4 minutes, about 5 minutes, about 6 minutes, about 7 minutes, about 8 minutes, about 9 minutes, about 10 minutes, or more in the mouth of a subject). In some embodiments, the composition is formulated as a mouthwash and has an extended residence time in the upper gastrointestinal tract of a subject (e.g., a residence time of greater than about 30 seconds in the mouth of a subject or more).

In some embodiments, the composition is formulated to have reduced systemic exposure in the subject, e.g., wherein the active ingredient (e.g., a gingerol (e.g., 6-gingerol), a shogaol (e.g., 6-shogaol), or a capsaicinoid (e.g., capsaicin)) has only minimal systemic exposure relative to the dosage amount. In some embodiments, the systemic exposure in a subject is measured through the concentration of the active ingredient (e.g., a gingerol (e.g., 6-gingerol), a shogaol (e.g., 6-shogaol), or a capsaicinoid (e.g., capsaicin)) in the blood, urine, or tissue (e.g., adipose tissue) of a subject. In some embodiments, the composition is formulated to have less than about 50% total systemic exposure relative to the dosage amount in a subject, e.g., wherein the systemic exposure is measured through the concentration of the active ingredient (e.g., a gingerol, a shogaol, or a capsaicinoid) in the blood, urine, or tissue (e.g., adipose tissue) of a subject. In some embodiments, the systemic exposure of the composition (e.g., the active ingredient of the composition (e.g., a gingerol (e.g., 6-gingerol), a shogaol (e.g., 6-shogaol), or a capsaicinoid (e.g., capsaicin))) is less than about 40%, less than about 30%, less than about 20%, less than about 10%, less than about 5%, less than about 1%, less than about 0.5%, less than about 0.1%, less than about 0.05%, less than about 0.01%, or lower relative to the dosage amount in a subject. In some embodiments, the composition of the present invention is formulated as to provide little to no systemic exposure in a subject.

In some embodiments, the compositions may be in the form of a dry powder, granule, tablet, or capsule that may be reconstituted in a specified amount of a liquid. The dried components may be mixed together and milled (e.g., to create a homogenous powder) or mixed in aqueous solution and dried by using methods known to one of skill in the art. Dried powders or granules may be “loose” or fashioned into tablets.

In other embodiments, the compositions of the present invention may be in the form of a gel or paste further comprising a humectant (e.g., glycerin, propylene glycol, lithium chloride, alpha hydroxy acids, diols, urea, quillaia, polyols, sugar alcohols (e.g., sorbitol, glycerol, xylitol, mannitol), glyceryl triacetate, or neoagarobiose), a gum (e.g., xanthan gum, guar gum), an abrasive (e.g., silica, (e.g., Zeodent®)), a plasticizer, an additive (e.g., a sweetener, preservative, buffering agent, penetration agent, surfactant, coloring agent, flavoring agent, cleaning agent, and the like) or a thickener (e.g., silica (e.g., Zeodent®)). These additional components may be present in the composition of the invention from about 0.5% to about 99% (e.g., about 0.5%, about 0.1%, about 0.5%, about 1%, about 5%, about 10%, about 20%, about 30% about 40%, about 50%, about 75%, about 90%, about 95%, or about 99%) by weight based on the total volume of the composition, though these components may be present in lower or higher concentrations.

The gel or paste may be further packaged on or within a delivery device such as a bioadhesive strip, patch, film, or may be provided for application directly to the oral cavity (e.g., mucosal surfaces (e.g., in the mouth, nose, or throat), teeth, gums, or lips). For example, a paste or gel can be packaged in a unit that contains between about 0.1 ounces to about 16 ounces of the paste or gel. For example, the packaging can contain about 0.1 ounces, about 0.25 ounces, about 0.5 ounces, about 1 ounce, about 2 ounces, about 3 ounces, about 4 ounces, about 5 ounces, about 6 ounces, about 7 ounces, about 8 ounces, about 9 ounces, about 10 ounces, about 11 ounces, about 12 ounces, about 13 ounces, about 14 ounces, about 15 ounces, or about 16 ounces.

To make pills containing the composition of the invention, the powdered ingredients are mixed together with a binding agent, such as acacia or tragacanth, and are then made into a plastic mass by incorporation of any liquid drugs and addition of an inert liquid. The resulting mass, known as a pill mass, is then rolled into spheres and coated with talc, gelatin, or sugar.

To make tablets, components of the products and compositions described herein (e.g., 6-gingerol, 6-shogaol, capsaicin, or trans-cinnamaldehyde) are mixed with suitable diluents, such as dextrin, lactose, salt, starch, or synthetic substances, designed to ensure disintegration of the tablet in the body. To prevent sticking in the machine, a lubricant such as liquid paraffin, stearic acid, talc, or a synthetic substance is usually added. Furthermore, it is essential that the tablet machines are fed with the drug mixture in a free-flowing form to ensure complete filling of the molds. To achieve this, the composition mixture is customarily granulated by mechanically forcing pellets of the mixture through a sheet of perforated-metal. The granulated mixture is fed into the tablet machine, which feeds the correct dose into a cavity, the mixture then being compressed by means of a punch that fits into the cavity. To be successful, the tablet maker must choose correct diluents and lubricants, prepare suitable granules, and obtain the right degree of compression in the tablet machine. Excessive compression may mean that the tablet will not disintegrate in the body; insufficient compression results in fragile tablets that may break, causing inaccurate dosage. Coatings of various types may be applied to the tablet to protect the ingredients from deterioration, to hide the taste of certain components, to control the release of the active components from the tablet, or to produce a more attractive tablet. For sugar coatings, a concentrated sucrose syrup containing suspended starch, calcium or magnesium carbonate, or other suitable substance is applied, each successive layer being dried before the application of the next. After the final layer is dried, it is highly polished to give an elegant finish. Sugar coatings provide both protection and a sweet taste. Film coatings can also be used, in which a very thin transparent film, usually a cellulose derivative, is applied. Enteric coating is designed to resist solution in the stomach and to dissolve in the more alkaline intestinal fluid. Many substances have been used for enteric coatings, one of which is cellulose acetate phthalate (cellacephate). In the manufacture of layered tablets, incorporating two or more drugs, a compressed tablet is fed to a second machine where another layer is compressed around it. In this way, drugs normally incompatible may be formulated in the same tablet.

Other solid dosages such as lozenges, troches, candies, dragées, or pastilles disintegrate or dissolve in the mouth, slowly releasing the active ingredient (e.g., 6-gingerol, 6-shogaol, capsaicin, or trans-cinnamaldehyde). The base usually consists of a mixture of sugar and gum or gelatin. Lozenges and troches are generally manufactured by compression techniques, while pastilles are fabricated by fusion and the use of molds. Dry extracts are prepared by the methods for fluid extracts, followed by evaporation, usually under reduced pressure, either to a pilular consistency or to dryness. Dry extracts are usually granulated by being passed through a sieve and may be used for the preparation of tablets.

Liquid dosage forms for oral administration include pharmaceutically acceptable emulsions, microemulsions, solutions, suspensions, syrups (e.g., syrup concentrates), linctuses, drops, mouthwash, and elixirs. In addition to the components of the products and compositions described herein, e.g., a gingerol (e.g., 6-gingerol), a shogaol (e.g., 6-shogaol), or a capsaicinoid (e.g., capsaicin), the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The compositions and solutions described herein may be bottled or packaged in, for example, glass bottles, plastic bottles and containers (e.g., polyethylene terephthalate or foil-lined ethylene vinyl alcohol), metal cans (e.g., coated aluminum or steel), lined cardboard containers, pouches, packs, wrappers, or any other packaging known to one of skill in the art. For example, a ready-to-drink beverage can be bottled or packaged in a unit that contains between about 10-1000 mL of the beverage. For example, the packaging can contain about 10 mL, 20 mL, 50 mL, 100 mL, 200 mL, 300 mL, 400 mL, 500 mL, 600 mL, 700 mL, 800 mL, 900 mL, or 1000 mL of the beverage. Alternatively, the packaging can contain 200 mL, 250 mL, 330 mL, 350 mL, 355 mL, 375 mL, 440 mL, or 500 mL of the beverage. A ready-to-drink beverage can also be bottled or packaged in a unit that contains between about 1-32 fluid ounces of beverage (e.g., the unit may contain about 1, 2, 5, 6.75, 8, 8.3, 8.4, 8.45, 9.6, 10, 12, 15, 15.5, 16, 18.6, 20, 23, 24, or 32 fluid ounces). Where a shelf-stable composition or solution is desired, the packaging is appropriately sterilized before being filled by the pasteurized, ultra-pasteurized, or sterilized composition or solution. Where required for mutual stability of two or more components (for example if a component is unstable at low pH), the packaging may feature multiple containers that can be mixed shortly before ingestion or that can be consumed serially.

Formulations for oral use may also be provided as chewable tablets, or as hard gelatin capsules wherein components of the products and compositions described herein (e.g., a gingerol (e.g., 6-gingerol), a shogaol (e.g., 6-shogaol), or a capsaicinoid (e.g., capsaicin)) are mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

### Oil-Based Formulations

The compositions of the invention can be formulated as an oil-based formulation for oral administration.

In one embodiment, the oil-based formulation includes a formulation base composition including an oil and a lipophilic additive, which can be solid or pasty at room temperature. The lipophilic additive can include waxes, fatty acid mono-, di- or triglycerides, fatty acids and polyethylene glycols and the polyethylene glycol fatty acid esters, as well as their mixtures and can be present in ranges from about 5 to 20% by weight or the composition (e.g., about 5%, about 6%, about 10%, about 15%, about 17%, about 18%, about 19%, or about 20%). The waxes can be beeswax, candelilla, wax, carnauba wax, polyethylene oxide wax or petroleum wax (or microcrystalline wax). The fatty acid mono-, di--, or triglycerides can have different degrees of esterification. The fatty acids can be selected from among palmitic acid, stearic acid, or behenic acid and their calcium, sodium, potassium or magnesium salts. The polyethylene glycols and fatty acid polyethylene glycol esters can have a molecular weight of between about 600 to 6000. The oil can include vegetable oils such as soya oil, sunflower oil, corn oil, olive oil or nut oil, and among the mineral oils such as liquid paraffin, as well as their mixtures. The oil-based formulations can be present in the form of a soft or hard capsule and can be prepared by traditional techniques known in the art. In one such technique, the lipophilic additive is incorporated into the oil which is heated at a temperature sufficiently high to melt the lipophilic additive completely and obtain a homogeneous mixture. After cooling to approximately 50° C., components of the products and compositions described herein (e.g., a gingerol (e.g., 6-gingerol), a shogaol (e.g., 6-shogaol), or a capsaicinoid (e.g., capsaicin)) are incorporated into this mixture with stirring. The mixture thus obtained is cooled to a temperature between 25 and 40° C., and optionally, soft or hard capsules are filled with this mixture. For a detailed discussion of lipids and lipid-based formulations see, for example, Porter et al., Nat Rev Drug Discov 2007, 6(3):231-248.

In another embodiment, components of the products and compositions described herein (e.g., a gingerol (e.g., 6-gingerol), a shogaol (e.g., 6-shogaol), or a capsaicinoid (e.g., capsaicin)) can be formulated as an oil for topical administration. Generally, the activators described herein at concentration ranges of about >20% to about 95% (w/w) are solubilized in a solvent capable of solubilizing the ion channel activator. Solvents that may be used include volatile solvents (e.g., methanol, ethanol, acetone, isopropanol, n-propanol, cyclohexane and alkanes with molecular weight less than dodecane (C12)), semi-volatile solvents (e.g., volatile essential oils such as clove oil, tea tree oil, sesame oil, and cineole), and non-volatile solvents (e.g., polyethylene glycol 400, Lutrol® (polyethylene polyoxypropylene block copolymer available from BASF), glyceryl monooleate, glycerin, lanolin, low melting waxes, sesquiterpenes and alkanes, alkenes, alkenoic and alkenoic acids>C28). The oils may further include a crystallization inhibitor, for example, polyvinylpyrrolidone, Luvitol® BD 10 P (BASF), povidone and its derivatives (e.g., crospovidone); dextrin derivatives, polyethylene glycol, polypropylene glycol, mannitol and glycerin, and mono and diglycerides of essential oils, polyglycerin fatty acid esters, sucrose palmitic acid ester, pentaerythritol ester of wood rosin (Pentalyn A®), and Eudagrits®. Crystallization inhibitors may range from about 0.1 to about 10% w/w. The oils of the ion channel activators described herein may be administered orally as an oil.

### Controlled Release Formulations

It is also within the scope of the invention to provide compositions that are formulated for modined release (e.g., delayed release, prolonged and/or slow release, extended release, or rapid release) of the components of the products and compositions described herein (e.g., a gingerol (e.g., 6-gingerol), a shogaol (e.g., 6-shogaol), or a capsaicinoid (e.g., capsaicin)), e.g., to reduce gastrointestinal side-effects. Such compositions are well-known in the art and include e.g., diffusion-controlled, drug delivery systems, osmotic pressure controlled drug delivery systems, or erodible drug delivery systems. Exemplary delivery systems are the SQZgel™ (MacroMed, Inc.), comprising a pH-sensitive polymer mixture combined with an outer coating in which the acidic environment of the stomach causes the polymer to imbibe with water and swell, entrapping the ion channel activator, e.g., a gingerol (e.g., 6-gingerol), a shogaol (e.g., 6-shogaol), or a capsaicinoid (e.g., capsaicin). Upon entering the higher pH of the intestines, the polymer slowly shrinks, or “squeezes” at a “dialed-in” rate releasing the active composition in a sustained manner); the Egalet® extrusion based technology (Egalet A/S), comprising a biodegradable coat and a matrix, including the ion channel activator (e.g., a gingerol (e.g., 6-gingerol), a shogaol (e.g., 6-shogaol), or a capsaicinoid (e.g., capsaicin)), which is surface erodible, hydrophobic, and composed of PEG-stearate); Diffucaps/Surecaps (small beads approximately 1 mm or less in diameter that can be incorporated into hard gelatin capsules, where the ion channel activator (e.g., a gingerol (e.g., 6-gingerol), a shogaol (e.g., 6-shogaol), or a capsaicinoid (e.g., capsaicin)) release profiles are created by layering drug onto a neutral core such as sugar spheres, crystals, or granules followed by a rate-controlling, functional membrane); and MeltDose®, which involves formulating solubilized, individual moelcules into tablets).

The components of the products and compositions described herein (e.g., a gingerol (e.g., 6-gingerol), a shogaol (e.g., 6-shogaol), or a capsaicinoid (e.g., capsaicin)) can be formulated for pH controlled release. Examples of suitable formulation principles are, for example, compositions provided with an enteric coating or hydrogels of a type described in U.S. Pat. Nos. 6,537,584 and 5,484,610, which are hereby incorporated by reference.

Another suitable formulation includes the formulation of components of the products and compositions described herein (e.g., a gingerol (e.g., 6-gingerol), a shogaol (e.g., 6-shogaol), or a capsaicinoid (e.g., capsaicin)) together with vitamin E concentrate in soft or hard gelatin capsules. Another specific example of a suitable formulation includes formulation of products and compositions described herein (e.g., a gingerol (e.g., 6-gingerol), a shogaol (e.g., 6-shogaol), or a capsaicinoid (e.g., capsaicin)) together with ethanol, tocoₕherolethylene glycol 1000 succinate (TPGS), corn oil, and wax in soft or hard gelatin capsules. Variations of this formulation can include ethanol, TPGS, corn oil, and polyglycolized glycerides (e.g., Gelucire) in soft or hard gelatin capsules. The resulting product can be a semi-solid or solid dosage form. The release rate of this formulation is dependent on degradation due to lipases in the intestine.

A further example of a suitable formulation is an oral pulsed dose drug delivery system. This dosage form can be perceived as a modified form of the Schering Repetab tablets. A portion of the composition of the present invention is put in the core of a tablet. The core can for example, be made by conventional wet granulation or continuous granulation such as extrusion followed by compaction of the granulate into tablets. The core is then coated using an appropriate technology, for example, by air-suspension using an enteric coating polymer such as Eudragits. The first releasing dose is compression coated on the core or air-suspension coated either with the enteric coat or on top of the enteric coat. In an embodiment of the invention, the first releasing dose is air-suspension coated with the enteric coat. In a further embodiment of the invention, the first releasing dose is compression coated on the core, in order to avoid release of the composition according to the invention prior to the degradation of the enteric coat, such degradation typically occurring at pH values higher than those found in the gastric ventricle the degradation of the enteric coat typically occurs after passage of the gastric ventricle). Another example of a suitable formulation is an oral sustained drug delivery system. In this delivery system, the core can for example, be made by conventional wet granulation or continuous granulation such as extrusion followed by compaction of the granulate into tablets. The core is then coated using appropriate technology, for example, by air-suspension using ethylcellulose and a hydrophilic excipient such as hydroxyl propyl cellulose (HPC).

In some embodiments, components of the products and compositions described herein (e.g., a gingerol (e.g., 6-gingerol), a shogaol (e.g., 6-shogaol), or a capsaicinoid (e.g., capsaicin)) are in the form of micro-crystals with hydrophilic surfaces. The micro-crystals can be film coated directly in order to achieve a sustained release formulation. The compositions of the invention can also be complexed with genuine cyclodextrins and cyclodextrin-derivatives (e.g., alkyl- and hydroxyalkyl-derivatives or sulfobutyl-derivatives). The complexation is achieved by methods known in the art. Complexation can lead to a higher solubility and a higher dissolution rate and higher bioavailability.

In other embodiments, the composition can include a pharmaceutically acceptable excipient that is an agent for delayed or controlled release of the components of the products and compositions described herein (e.g., a gingerol (e.g., 6-gingerol), a shogaol (e.g., 6-shogaol), or a capsaicinoid (e.g., capsaicin)). In some aspects, the agent is a water-soluble polymer, including but not limited to, hydroxypropylmethyl cellulose, hydroxypropyl cellulose, hydroxypropyl methyl cellulose (HPMC), methyl cellulose, ethyl cellulose, or carboxymethyl cellulose.

The components of the products and compositions described herein (e.g., a gingerol (e.g., 6-gingerol), a shogaol (e.g., 6-shogaol), or a capsaicinoid (e.g., capsaicin)) can be targeted to the mucus/mucosal lining of the mouth, tongue, nose, or gastrointestinal tract (GIT) through the use of bioadhesives. A bioadhesive is defined as a synthetic or biological material which is capable of adhering to a biological substrate or tissue. When the biological substrate is mucus, the term “mucoadhesive” has been employed. When the biological tissue involved is the mouth or the stomach, the terms “buccoadhesive” or “gastroadhesive” have been employed. Bioadhesives can remain attached to the biological substrate for an extended period of time. The period of time a bioadhesive is required to remain attached to a biological substrate will vary according to the target site and the condition being treated. Other delivery systems that can target the TRP or ASIC channel activators, (e.g., a gingerol (e.g., 6-gingerol), a shogaol (e.g., 6-shogaol), or a capsaicinoid (e.g., capsaicin)) described herein to the colon include, but are not limited to:
(a) covalent linkage of the components of the products and compositions described herein (e.g., a gingerol (e.g., 6-gingerol), a shogaol (e.g., 6-shogaol), or a capsaicinoid (e.g., capsaicin)) with the carrier to form a prodrug that is stable in the stomach and small intestine and releases the ion channel activator in the large intestine upon enzymatic transformation by the intestinal microflora; examples of these prodrugs include azo-conjugates, cyclodextrin-conjugates, glycoside-conjugates, glucuronate conjugates, dextran-conjugates, polypeptide and polymeric conjugates;
(b) approaches to deliver intact molecule to the colon, such as coating with pH-sensitive polymers to release components of the products and compositions described herein (e.g., a gingerol (e.g., 6-gingerol), a shogaol (e.g., 6-shogaol), or a capsaicinoid (e.g., capsaicin)) at neutral to alkaline pH, or coating with biodegradable polymers which release the ion channel activator upon degradation by the bacteria in the colon;
(c) embedding the components of the products and compositions described herein (e.g., a gingerol (e.g., 6-gingerol), a shogaol (e.g., 6-shogaol), or a capsaicinoid (e.g., capsaicin)) in biodegradable matrices and hydrogels which release the ion channel activator (e.g., a gingerol (e.g., 6-gingerol), a shogaol (e.g., 6-shogaol), or a capsaicinoid (e.g., capsaicin)) in response to the pH or biodegradation;
(d) time released systems where once the multicoated formulation passes the stomach, the components of the products and compositions described herein (e.g., a gingerol (e.g., 6-gingerol), a shogaol (e.g., 6-shogaol), or a capsaicinoid (e.g., capsaicin)) are released after a lag time of 3-5 hrs which is equivalent to the transit time of the small intestine;
(e) using redox-sensitive polymers where a combination of azo and disulfide polymers provide release of the components of the products and compositions described herein (e.g., a gingerol (e.g., 6-gingerol), a shogaol (e.g., 6-shogaol), or a capsaicinoid (e.g., capsaicin)) in response to the redox potential of the colon;
(f) osmotic controlled delivery where the components of the products and compositions described herein (e.g., a gingerol (e.g., 6-gingerol), a shogaol (e.g., 6-shogaol), or a capsaicinoid (e.g., capsaicin)) are released through semi-permeable membrane due to osmotic pressure.

### Micro and Nanoparticle Formulations

In one embodiment, components of the products and compositions described herein (e.g., a gingerol (e.g., 6-gingerol), a shogaol (e.g., 6-shogaol), or a capsaicinoid (e.g., capsaicin)) are prepared as loaded nano- and micro-particles for sustained release and are formulated by the nano-precipitation or the oil-in-water single emulsion solvent evaporation/extraction method. First, the specific component (e.g., a gingerol (e.g., 6-gingerol), a shogaol (e.g., 6-shogaol), or a capsaicinoid (e.g., capsaicin)) are prepared as loaded poly(lactic-co-glycolic acid) (PLGA) nanoparticles by the nano-precipitation method. The volume of oil-water ratio can be adjusted (e.g., from about 1:2 to 1:5, e.g., about 1:2, 1:3, 1:4, or 1:5), and size of the nanoparticles can be selected (e.g., from about 162+/−3 nm to 153+/−3 nm, e.g., about 154, 155, 156, 157, 158, 159, 160, 161, or 162) to increase drug loading efficiency and drug release period. To get a more sustained release, a modified single emulsion method can be applied with biocompatible polymers such as polylactic acid (PLLA), polyhydroxy butyrate (PHB), polyglycolic acid (PGA), PLGA, and poly-ε-caprolactone (PCL).

In another embodiment, stomach specific mucoadhesive nanoparticles (SSMN) can be used to improve controlled delivery of components of the products and compositions described herein (e.g., a gingerol (e.g., 6-gingerol), a shogaol (e.g., 6-shogaol), or a capsaicinoid (e.g., capsaicin)) by continuous release of the activator for a prolonged period to its absorption site to ensure optimal bioavailability. The components of the products and compositions described herein (e.g., a gingerol (e.g., 6-gingerol), a shogaol (e.g., 6-shogaol), or a capsaicinoid (e.g., capsaicin)) with a narrow absorption window are mostly associated with improved absorption at the jejunum and ileum due to enhanced absorption properties of these sites (e.g., large surface area), or because of enhanced solubility in the stomach as opposed to the more distal parts of the gastrointestinal tract. Ion channel activators (e.g., a gingerol (e.g., 6-gingerol), a shogaol (e.g., 6-shogaol), or a capsaicinoid (e.g., capsaicin)) that may benefit from using stomach specific mucoadhesive nanoparticles includes those that act locally in the stomach, those with low solubility at high pH values, those that are primarily absorbed in the stomach, those with a narrow window of absorption, e.g., ion channel activators (e.g., a gingerol (e.g., 6-gingerol), a shogaol (e.g., 6-shogaol), or a capsaicinoid (e.g., capsaicin)) that are absorbed mainly from the proximal part of the small intestine, those that absorb rapidly from the gastro intestinal tract, those that degrade in the colon, and those that are unstable in intestinal fluids. Longer residence time in the stomach could be advantageous for local action especially in the upper part of the small intestine.

### Routes of Administration

The compositions described herein may be administered to a subject in a variety of forms depending on the selected route of administration, as will be understood by those skilled in the art and as relating to the particular disease or condition to be treated. The compositions used in the methods described herein may be administered, for example, by topical, enteral, or parenteral applications. Topical applications include but are not limited to epicutaneous, inhalation, enema, eye drops, ear drops, and applications through mucous membranes in the body. Enteral applications include oral administration, rectal administration, vaginal administration, and gastric feeding tubes. Parenteral administration includes intravenous, intraarterial, intracapsular, intraorbital, intracardiac, intradermal, transtracheal, subcuticular, intraarticular, subcapsular, subarachnoid, intraspinal, epidural, intrastemal, intraperitoneal, subcutaneous, intramuscular, transepithelial, nasal, intrapulmonary, intrathecal, rectal, and topical modes of administration. Parenteral administration may be by continuous infusion over a selected period of time.

In some embodiments of the current invention, components of the products and compositions described herein (e.g., a gingerol (e.g., 6-gingerol), a shogaol (e.g., 6-shogaol), or a capsaicinoid (e.g., capsaicin)) are administered through the oral cavity to achieve mucosal and transmucosal effects. Exemplary applications include buccal, nasal, intradermal, inhalational, topical, subcutaneous, sublingual, sublabial, and insufflation administrations. Compositions of the current invention may include a penetration enhancer to increase the bioavailability of the ion channel activator within the oral cavity. Exemplary penetration enhances include surfactants (e.g., anionic surfactants (e.g, sodium lauryl sulfate), cationic surfactants (e.g., cetyl pyridinium chloride), and nonionic surfactants (e.g., poloxamer, Brij, Span, Myrj, Tween)), bile salts (e.g., sodium glycocholate, sodium taurodeoxycholate, sodium taurocholate), fatty acids (e.g., oleic acid, caprylic acid, lauric acid, lysophosphatidylcholine, phosphatidylcholine), cyclodextrins (e.g, α-, β-, or γ-cyclodextrans, methylated cyclodextrins), chelators (e.g., EDTA, citric acid, sodium salicylate, methyl salicylates), polymers (e.g., positively charged polymers (e.g., chitosan, trimethyl chitosan)), and cationic compounds (e.g., poly L-arginine, L-lysine).

For intravenous or intrathecal delivery or direct injection, the composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

The choice of the route of administration will depend on whether a local or systemic effect is to be achieved. For example, for local effects, the composition can be formulated for topical administration and applied directly where its action is desired. For systemic, long term effects, the composition can be formulated for enteral administration and given via the digestive tract. For systemic, immediate and/or short term effects, the composition can be formulated for parenteral administration and given by routes other than through the digestive tract.

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection moldings are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

### Dosage

The compositions of the present invention are formulated into acceptable dosage forms by conventional methods known to those of skill in the art. Actual dosage levels of the active ingredients in the compositions of the present invention may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the subject being treated, and like factors well known in the medical arts. A physician or veterinarian having ordinary skill in the art can readily determine and prescribe the effective amount of the composition required. For example, the physician or veterinarian can start doses of the substances of the invention employed in the composition at levels lower than that required in order to achieve the desired therapeutic effect and gradually increase the dosage until the desired effect is achieved. In general, a suitable daily dose of a composition of the invention will be that amount of the substance which is the lowest dose effective to produce a therapeutic effect. Such an effective dose will generally depend upon the factors described above. Preferably, the effective daily dose of a therapeutic composition may be administered as one, two, three, four, five, six or more sub-doses administered separately at appropriate intervals throughout the day, optionally, in unit dosage forms.

The frequency of treatment may also vary. The subject can be treated one or more times per day (e.g., once, twice, three, four or more times) or every so-many hours (e.g., about every 2, 4, 6, 8, 12, or 24 hours). The composition can be administered 1, 2, or 3 times per 24 hours. The time course of treatment may be of varying duration, e.g., for two, three, four, five, six, seven, eight, nine, ten, or more days, two weeks, 1 month, 2 months, 4 months, 6 months, 8 months, 10 months, or more than one year. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Foodstuff and Food Supplements

The present invention also relates to the use of the composition as foodstuff, food supplement or dietetic product (a foodstuff intended for a particular diet). In particular, the composition can be incorporated into foodstuffs which are industrially produced or craftsmen-prepared, such as oils, butter, margarine, bread spreads, or baked goods. It can also be presented in the form of a powder for dilution in water or food bars.

The composition of the invention can further be administered in combination with a dietary supplement to promote and/or maintain general health. Examples of dietary supplements include, but are not limited to, a vitamin (e.g., Vitamin A, Vitamin B₁, B₂, B₃, B₅, B₆, B₇, B₉, B₁₂, Vitamin C, Vitamin D, Vitamin E, and Vitamin K), a mineral (e.g., potassium, chlorine, sodium, calcium, magnesium, phosphorus, zinc, iron, manganese, copper, iodine, selenium, and molybdenum), an herb or botanical (e.g., St. John's-wort, kava, Shilajit, and Chinese herbal medicines), an amino acid (e.g., glycine, serine, methionine, cysteine, aspartic acid, glutamic acid, glutamine, tryptophan, and phenylalanine), and a concentrate, constituent, extract, and/or a combination of any of the above.

### Methods of Evaluating a Subject

The present invention provides various methods for evaluating a subject for the efficacy of a muscle cramp treatment or for treating an unwanted or abnormal muscle contraction, e.g., a muscle cramp, spasticity, dystonia, or fasciculation. In the methods described herein, a test muscle of a subject is electrically induced to have a test muscle cramp, and the electrical activity of the test muscle is recorded. In some embodiments, a test aliquot of a composition for treating muscle cramps, spasms, dystonias, or fasciculations is administered to the subject, and a second test muscle cramp is induced. The electrical activity of the second test cramp is also recorded. Comparison and analysis of the recordings of the first and second test cramp can indicate the efficacy of the test aliquot on reducing, alleviating, or preventing the cramp. Comparison and analysis of the recordings can also be used to classify subjects, or identify subjects for certain treatments for muscle cramps.

Alpha motor neurons project from the brainstem and the spinal cord and innervate the muscles. Stimulation of the alpha motor neurons results in transmittal of an electrical signal to the muscles, generated from the movement of ions across the cell membrane. The electrical stimulation from the motor neurons causes muscle movement or contraction, e.g., a muscle cramp or spasm. When the muscles are at rest, there is minimal or no electrical signal. Activity of the alpha motor neurons can be modulated by signaling from primary sensory neurons, which are activated by sensory input. Stimulation of non-taste primary sensory neurons with nerve endings in the mouth, esophagus and stomach, e.g., through activation of specific ion channels, can induce cause upregulation of inhibitory signals to the alpha motor neurons. Through this mechanism of interneuronal negative feedback, activation of primary sensory neurons inhibit or prevents alpha motor neuron firing via inhibitory signaling, and thereby inhibits muscle contractions of muscle cramps or spasms.

Electrical stimulation can elicit a muscle contraction that recapitulates a muscle cramp, spasm, dystonia, or fasciculation. In some embodiments, in the methods described herein, electrical stimulation is used to induce a test muscle cramp. The electrical activity of the test muscle before, during, and after the test muscle cramp is detected, measured, and recorded. Analysis of the recording of the electrical activity of a muscle experiencing an electrically induced cramp can be useful for determining the efficacy of a treatment for alleviating a muscle cramp.

Electromyography is a technique for measuring and recording the electrical activity of a muscle during rest and movement. The instrument that detects and records the electric signal generated from a muscle is called an electromyograph. The recording of the electrical activity obtained by the electromyograph is known as an electromyogram (myogram, or EMG). The eletromyograph may comprise at least one recording electrode, at least one reference electrode, an amplifier unit, a device for converting the analog signals to digital signals, and a device for generating and displaying the recordings. Optionally, the instrument may also include at least one mechanism to detect additional biofeedback, such as body or skin temperature by a skin thermistor.

The electrical activity of a muscle, e.g., a test muscle, is recorded and detected by an electrode, or a lead. Electrodes may be placed either on the surface of the skin (e.g., surface electrodes) or inserted into the muscle or body cavity (e.g., inserted electrodes). Surface electrodes are non-invasive and are placed on the skin. Inserted electrodes include needle and fine wire electrodes, and are inserted directly into the muscle tissue. In a preferred embodiment, the electrode used in the present invention is a surface electrode.

A recording electrode is preferably placed over a test muscle, and a reference electrode is placed nearby, e.g., within 2-6 inches of an active electrode. Preferably, the reference electrode is placed on a synergistic muscle. A synergistic muscle is a muscle that aids or participates in movement with the test muscle but does not cramp with the test muscle when electrically induced. In some preferred embodiments, a series or an array of multiple recording electrodes is used. For example, a linear array of 8 recording electrodes is applied on the test muscle, and optionally, a linear array of 4-8 recording electrodes is used on the synergistic muscle. In other embodiments, a grid array of recording electrodes is applied on the target muscle, e.g., a grid array of 6×5 electrodes is used.

In the methods described herein, electrical stimulus is applied to a test muscle to induce a test muscle contraction. The electrical stimulus is delivered by a stimulation electrode. The stimulation electrode is placed preferably directly on the skin over the test muscle. The electrical stimulus is defined by multiple parameters, such as stimulation frequency (Hertz or Hz), current intensity (milliamps or mA), pulse frequency (pulse per second or pps), and duration of time of stimulation. The electrical stimulus can be delivered by percutaneous electrical stimulation or surface electrical stimulation.

Preferred test muscles for electrically inducing a muscle cramp include the flexor hallucis brevis (FHB, or big toe flexor muscle) and gastrocnemius (calf muscle). Other target muscles may include the abductor hallucis (AH), the biceps brachii (biceps), the triceps surae (triceps), or the quadriceps femoris (quadriceps).

The appropriate stimulation frequency of electrical stimulation to induce a test muscle cramp may vary depending on the size or location of the muscle or the individual. For example, the electrical stimulation can be at least 1 Hz, at least 2 Hz, at least 3 Hz, at least 4 Hz, at least 5 Hz, at least 6 Hz, at least 7 Hz, at least 8 Hz, at least 9 Hz, at least 10 Hz, at least 11 Hz, at least 12 Hz, at least 13 Hz, at least 14 Hz, at least 15 Hz, at least 20 Hz, at least 25 Hz, at least 30 Hz, at least 35 Hz, at least 40 Hz, at least 50 Hz, at least 60 Hz, at least 70 Hz, at least 80 Hz, at least 90 Hz, or at least 100 Hz. In some preferred embodiments, the stimulation frequency for the FHB is 8 Hz, 10 Hz, 12 Hz, 14 Hz, or 18 Hz. In some embodiments, the stimulation frequency can vary over time. For example, the stimulation frequency increases over time, e.g., from 2 Hz to 24 Hz, increasing by 2 Hz increments.

The minimum frequency of the electrical stimulation capable of inducing a cramp has been termed the “threshold frequency.” Studies have shown that the threshold frequency for cramp induction is lower in cramp-prone subjects compared with subjects with no history of cramps. (Bertolasi et al. Ann Neurol (1993), 133:303-6; Miller and Knight, Muscle Nerve (2009) 39:364-8; and Minetto et al, Muscle Nerve (2009) 40:535-44). For example, Miller and Knight (Muscle Nerve (2009) 39:364-8) found a threshold frequency for the flexor hallucis brevis muscle of approximately 15 Hz in subjects with a history of cramping and of approximately 25 Hz in individuals not prone to cramping.

The amplitude, or intensity of the current, of the electrical stimulus also may vary depending on the size or location of the muscle or the individual. A maximal current intensity refers to the current intensity required to achieve a plateau in the M-wave peak amplitude. The M-wave refers to the EMG signal detected. In a preferred embodiment, the current intensity is 30% supramaximal, or greater than, the maximal current intensity. In some embodiments, the maximal current intensity is determined for each individual subject prior to testing the subject. In some embodiments, the current intensity is 5 mA, 10 mA, 15 mA, 20 mA, 25 mA, 30 mA, 40 mA, 50 mA, or 60 mA.

The electrical stimulus can be applied as a series of pulses for a particular duration of time. In some embodiments, the stimulus may be applied as a series of at least 100 microsecond pulses, at least 120 microsecond pulses, at least 150 microsecond pulses, at least 180 microsecond pulses, at least 200 microsecond pulses, at least 300 microsecond pulses, at least 400 microsecond pulses, at least 500 microsecond pulses, or at least 600 microsecond pulses. In some embodiments, the stimulus may be applied for 1 second, 2 seconds, 3 seconds, 4 seconds, 5 seconds, 6 seconds, 7 seconds, 8 seconds, 9 seconds, 10 seconds, or longer. Preferably for inducing cramps in the FHB, the stimulus is applied as a series of 180 microsecond pulses for 7 seconds.

In some embodiments, the parameters of electrical stimulation to be applied to the test muscle may be adjusted to decrease or increase the magnitude of the test muscle contraction to better recapitulate a muscle cramp, spasm, dystonia, or fasciculation. In some embodiments, the frequency or intensity of electrical current applied to the test muscle may vary depending on the type of test muscle contraction desired, e.g., the frequency of stimulation is increased to induce a test muscle cramp compared to a test muscle spasm.

In some embodiments, in the methods described herein, the electrical activity of a target muscle is detected and recorded by a recording electrode before, during, and after an induced cramp. The electrical activity is recorded and displayed as a profile or electromyogram (EMG). Preferably, the profile contains the electrical activity before, during, and after the application of electrical stimulation to induce a muscle cramp. In some embodiments, the profile contains the electrical activity during the application of electrical stimulation and after the application of electrical stimulation. In some embodiments, the electrical activity is converted to root mean square (RMS) values and is displayed as a function of time. In the case where more than one recording electrode is utilized to measure the electrical activity of the target muscle, the profile contains the average electrical activity detected from all of the recording electrodes as a function of time.

The pattern of the recorded electrical activity or signal can indicate the presence or absence of an induced muscle cramp. Indication of an induced muscle cramp includes involuntary electrical signal of the stimulated muscle after cessation of the electrical stimulation, preferably with concurrent absence of electrical signal of the synergistic muscle. Alternatively, a signal amplitude greater than 2 or 3 standard deviations above the 1 second baseline signal amplitude of either the target muscle prior to stimulation or the synergistic muscle after stimulation indicates an induced cramp. In some embodiments, the induced muscle cramp can last for at least 5 seconds, at least 10 seconds, at least 15 seconds, at least 20 seconds, at least 25 seconds, at least 30 seconds, at least 35 seconds, at least 40 seconds, at least 45 seconds, at least 50 seconds, at least 55 seconds, at least 1 minute, at least 2 minutes, at least 3 minutes, at least 4 minutes, or at least 5 minutes.

In some embodiments, a first muscle cramp is electrically-induced in a subject prior to administration of a composition for treating a muscle cramp, and after a rest period, a second muscle cramp is electrically-induced in a subject after administration of the composition. The profile obtained for the first induced cramp before administration of the composition is referred to as the reference profile. The profile obtained for the second induced cramp after administration of the composition is referred to as the treatment profile. Comparison of the treatment profile and the reference profile can be used to select subjects for treatment with the composition or to identify subjects that will be responsive to the treatment with the composition. Specifically, the properties of the induced cramp in the reference and treatment profiles are compared to determine whether the composition administered reduced or prevented the second induced cramp.

Alternatively, a value can be determined from the reference profile, e.g., a reference value, and a value can be determined from the treatment profile, e.g., a treatment value. The reference value and the treatment value can be compared to determine a reduction or prevention of a cramp. In some embodiments, the values determined from the reference and/or treatment profile represent the parameters of muscle cramp. A decrease in the treatment value compared to the reference value indicates that that the test aliquot administered is effective at alleviating or preventing a muscle cramp.

The parameters of a muscle cramp can be compared for determining the efficacy of a treatment for alleviating a cramp, e.g., by a reduction of a muscle cramp parameter or prevention of a cramp. Parameters of muscle cramps that can be determined from the electromyogram include the area under the curve, the peak amplitude, the duration of the cramp, and a change in threshold frequency used to elicit a test muscle cramp.

An area under the curve value can be calculated for the reference and treatment profile using standard methods known in the art. A decrease or absence of an area under the curve value in the treatment profile compared to the reference profile indicates that an electrically induced cramp has been reduced or prevented. In some embodiments, the area under the curve in the treatment profile may be at least 1%, 5%, 10%, 15,%, 20%, 25%, 30%, 35%, or 50% decreased compared to the area under the curve in the reference profile.

The peak amplitude after cessation of the electrical stimulus can be compared between the reference and treatment profiles. A reduction in or absence of the peak amplitude in a treatment profile compared to a reference profile indicates that an electrically induced cramp has been reduced or prevented. For example, the peak amplitude in a treatment profile may be at least 1%, 5%, 10%, 15,%, 20%, 25%, 30%, 35%, or 50% decreased compared to the peak amplitude of the reference profile.

The duration of the test cramp can be compared between the reference and treatment profile. A decrease in or absence of the duration of the cramp indicates the reduction or prevention of the electrically induced cramp. For example, the duration of the cramp may be at least 1%, 5%, 10%, 15,%, 20%, 25%, 30%, 35%, or 50% decreased compared to the duration of the cramp in the reference profile.

Threshold frequency refers to the minimum frequency of electrical stimulation required to elicit a cramp. In an embodiment, a change in the threshold frequency required to elicit a test muscle cramp indicates the efficacy of a treatment for alleviating a cramp. For example, a change in the threshold frequency may be at least 1%, 5%, 10%, 15,%, 20%, 25%, 30%, 35%, or 50% , 100%, 200% greater compared to the threshold frequency required before treatment, e.g., in the reference profile. In some embodiments, a change in the threshold frequency may be at least 1%, 5%, 10%, 15,%, 20%, 25%, 30%, 35%, or 50% , decreased compared to the threshold frequency required before treatment, e.g., in the reference profile.

In certain embodiments, the rest period between the first test cramp and the second test cramp is at least 1 minute, 2 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, 2 hours, 3 hours, 4 hours, 5 hours, 6 hours, 7 hours, 8 hours, 9 hours, 10 hours, 11 hours, 12 hours, 16 hours, 20 hours, or 24 hours. In certain embodiments, the electrical stimulation is re-applied to induce a second muscle cramp at least 1 minute, 2 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, 2 hours, 3 hours, 4 hours, 5 hours, 6 hours, 7 hours, 8 hours, 9 hours, 10 hours, 11 hours, 12 hours, 16 hours, 20 hours, or 24 hours after administration of the composition for treating muscle cramps.

In one embodiment, the test comprises determining that a cramp can be induced in a subject by application of stimulus. For example, the stimulus is percutaneous electrical stimulation or surface electrical stimulation. In some embodiments, the magnitude of the parameters of the induced muscle cramp, e.g., as determined from an EMG profile, identifies or classifies subjects with respect to selection of treatment regimens, or predicts the response of a subject to a specific treatment regimen.

### Methods of Treatment

The compositions of the invention may be useful for treating unwanted or abnormal muscle contractions, as well as any of the conditions and disorders described herein.

### Muscle Conditions and Disorders

The compositions and methods described herein include prevention or treatment of a subject having been diagnosed with or identified as having a muscle condition or disorder. Exemplary disorders include nocturnal cramps (e.g., nocturnal foot cramps or nocturnal leg cramps), multiple sclerosis, dystonia, spinal cord spasticity, neuromuscular disorders, including muscle pain and cramping associated with a neuromuscular disorder, a neurological condition (e.g., a peripheral nervous system condition or a central nervous system condition), a neurological injury (e.g., a central nervous system injury (e.g., spinal cord injury, brain injury, or stroke), muscle cramps, muscle spasms, and fasciculations.

The compositions and methods disclosed herein are suitable for the treatment or alleviation of an abnormal or unwanted muscle contraction or an absence of a normal muscle contraction. An abnormal or unwanted muscle contraction includes, e.g., a muscle cramp, a muscle spasm, muscle spasticity, a dystonia, or a fasciculation. An absence of a normal muscle contraction is associated with gait abnormalities, e.g., “foot drop”. The abnormal or unwanted muscle contraction may occur in a skeletal muscle or in a muscle that is not a skeletal muscle. The muscle that is not a skeletal muscle is a smooth muscle (also known as involuntary muscle).

Involuntary muscles, or smooth muscles, of various organs, such as the uterus, blood vessel wall, bowels, bile and urine passages, and bronchial tree, are also subject to cramps. In some embodiments, the methods and compositions described herein are also suitable for treating or evaluating a patient diagnosed with or having abnormal muscle contractions of a smooth muscle. Abnormal muscle contractions of the smooth muscle can include, but are not limited to, primary or secondary dysmenorrhea (e.g., menstrual cramps), diaphragmatic cramps or spasms, bladder spasms (e.g., incontinence), achalasia, and bowel cramps.

In some embodiments, the compositions of the invention are also useful for treating painful muscle contraction of the head or neck as in tension, cluster or migraine headache, back spasms, leg cramps due to spinal stenosis, skeletal muscle pain, muscle pain (e.g., fibromyalgia) and spasms (e.g., nocturnal cramps), back pain (e.g., low back pain), neuropathic pain, spasms and cramps due to treatment with dialysis, diuretics, β-blockers, statins, fibrates, β2-agonists, ACE inhibitors, ARBs and anti-psychotic medications, muscle claudication pain due to inactivity or restriction as seen in “economy class syndrome”, paralysis, peripheral artery disease or immobilization, and neuromuscular diseases.

In some embodiments, the compositions of the inventions are also useful for treating exercise-associated muscle cramps. Exercise-associated muscle cramps are muscle cramps or spasms that occur during or immediately following exercise. In some embodiments, the exercise-associated muscle cramps may begin during exercise and continue after the exercise has ceased. In some embodiments, the exercise-associated muscle cramps may be influenced (e.g., enhanced) by other conditions, such as dehydration, electrolyte imbalance, muscle fatigue, or another condition or disorder.

Neuromuscular disease can be caused by circulatory problems, stroke, immunological and autoimmune disorders, the failure of the electrical insulation surrounding nerves myelin, genetic/hereditary disorders, such as Huntington's disease, certain rare tumors, the failure of the connections between the nerves and the muscle fibers, and exposure to pernicious environmental chemicals, poisoning (e.g., heavy metal poisoning). Some neuromuscular diseases are caused either by viral infections or by attack by little-known pernicious proteins called prions. Diseases of the motor end plate include myasthenia gravis, a form of muscle weakness due to antibodies to the acetylcholine receptor, and its related condition Lambert-Eaton myasthenic syndrome (LEMS). Tetanus and botulism are bacterial infections in which bacterial toxins cause increased or decreased muscle tone, respectively. Myopathies are all diseases primarily resulting in muscular degeneration, rather than affecting the nerves themselves (e.g., nemaline myopathy, centronuclear myopathy, mitochondrial myopathies, inflammatory myopathies, familial periodic paralysis, or drug-induced myopathyies). Muscular dystrophies, including Duchenne's and Becker's, are a large group of diseases, many of them hereditary or resulting from genetic mutations, where the muscle integrity is disrupted. They lead to progressive loss of strength and decreased life span. Also within the scope of the invention is treatment of Guillain-Barre syndrome and inflammatory muscle disorders, such as polymyalgia rheumatic, polymyositis, dermatomyositis, inclusion body myositis, and rhabdomyolysis. Additional neuromuscular disorders include, but are not limited to, multiple sclerosis, spinal cord spasticity, spinal muscle atrophy, myasthenia gravis, spinal cord injury, traumatic brain injury, cerebral palsy, hereditary spastic paraplegia, motor neuron disease (e.g., amyotrophic lateral sclerosis, primary lateral sclerosis, progressive muscular atrophy, progressive bulbar palsy, pseudobulbar palsy, spinal muscular atrophy, progressive spinobulbar muscular atrophy (e.g., Kennedy's disease), or post-polio syndrome), neuralgia, fibromyalgia, Machado-Joseph disease, cramp fasciculation syndrome, carpal tunnel syndrome, acrodynia, neurofibromatosis, neuromyotonias (e.g., focal neuromyotonia, Isaacs' syndrome), peripheral neuropathy, piriformis syndrome, plexopathy (e.g., Brachial plexopathy or Lumbosacral plexobathy), radiculopathy (e.g., lower lumbar radiculopathy), and encephalitis. In some embodiments, a composition or method provided herein are administered to treat a symptom of a neuromuscular disease, e.g., a muscle cramp, muscle spasm, muscle spasticity, muscle pain, or muscle soreness associated with a neurological disease described herein.

Dystonia is a neurological condition that affects a muscle or group of muscles in a specific part of the body causing involuntary muscular contractions and abnormal postures. Types of dystonia include: focal dystonia, multifocal dystonia, segmental dystonia, generalized dystonia (e.g., torsion dystonia or idiopathic torsion dystonia), hemidystonia, blepharospasm, psychogenic dystonia, cervical dystonia, acute dystonic reaction, and vegetative-vascular dystonia. The methods and compositions described herein may be useful for cervical dystonia, cranial dystonia, laryngeal dystonia, and hand dystonia. Causes for the disorder include, but are not limited to, inheritance, birth-related or physical trauma, infection, poisoning (e.g., lead poisoning), or reaction to some pharmaceutical agents, e.g., neuroleptics. Treatment is difficult and has been limited to minimizing symptoms of the disorder, such as muscle cramping. The methods and compositions described herein may be useful for treatment of focal dystonia, blepharospasm, cervical dystonia, cranial dystonia, laryngeal dystonia, and hand dystonia. In some embodiments, improvement of symptoms relating to a dystonia (e.g., a cervical dystonia) may be measured by the Toronto Western Spasmodic Torticollis Rating Scale, the Tsui score, or the Oropharyngeal Swallow Efficiency Test, the Insomnia Severity Index Sleep Survey, and the Epworth Sleepiness Scale. In some embodiments, improvement of symptoms relating to a dystonia (e.g., a cervical dystonia) are improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more upon treatment with a composition of the present invention, e.g., as measured by the Toronto Western Spasmodic Torticollis Rating Scale, the Tsui score, or the Oropharyngeal Swallow Efficiency Test.

In one embodiment, the subject has been diagnosed or identified as having multiple sclerosis. Multiple sclerosis (MS) is also known as disseminated sclerosis and encephalomyelitis disseminate. MS is an inflammatory disease in which the insulating sheaths of nerve cells in the brain and spinal cord are damaged, thereby disrupting the ability of the nervous system to communicate. The three main characteristics of MS are the formation of lesions in the central nervous system (also called plaques), inflammation, and the destruction of myelin sheaths of neurons. Symptoms of MS can include muscle spasms and muscle weakness. In some embodiments, the subject has been diagnosed with MS and is further suffering from spasticity, muscle spasms, and muscle cramps. In some embodiments, improvement of symptoms relating to a MS and MS-related spasticity, muscle spasms, and muscle cramps may be measured by the Modified Ashworth Scale, the Tardieu Scale, the Numerical Rating Scale, the Barthel Activities of Daily Living Scale, the Timed 25-Foot Walk Test, the Insomnia Severity Index Sleep Survey, the Epworth Sleepiness Scale, the Clinical Global Impression (CGI) Scale, Quality of Life questionnaires (e.g., 36-Item Short Form Survey (e.g., SF-36) or the Multiple Sclerosis Spasticity Scale (MSSS-88) or the Medical Outcomes Study-Sleep Scale. In some embodiments, improvement of symptoms relating to MS and MS-related spasticity, muscle spasms, and muscle cramps are improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more upon treatment with a composition of the present invention, e.g., Modified Ashworth Scale, the Tardieu Scale, the Numerical Rating Scale, the Barthel Activities of Daily Living Scale, the Timed 25-Foot Walk Test, the Insomnia Severity Index Sleep Survey, the Epworth Sleepiness Scale, the Clinical Global Impression (CGI) Scale, Quality of Life questionnaires (e.g., 36-Item Short Form Survey (e.g., SF-36) or the Multiple Sclerosis Spasticity Scale (MSSS-88) or the Medical Outcomes Study-Sleep Scale.

In one embodiment, the subject has been diagnosed or identified as having motor neuron disease, such as Amyotrophic Lateral Sclerosis (ALS) or Primary Lateral Sclerosis (PLS). ALS is a disease involving both lower motor neuron cell bodies as well as upper motor neuron descending tracts in the spinal cord, and PLS is a disease involving only the latter. Symptoms of ALS and PLS can include muscle cramps/spasms and spasticity. In some embodiments, the subject has been diagnosed with ALS or PLS and is further suffering from spasticity, muscle spasms, and muscle cramps. In some embodiments, improvement of symptoms relating to a ALS- or PLS-related spasticity, muscle spasms, and muscle cramps may be measured by cramp frequency and/or cramp severity as assessed by a Visual Analogue Scale or Numerical Rating Scale for Pain, the Modified Ashworth Scale, the Tardieu Scale, a Spasticity Numerical Rating Scale, the ALS Assessment Questionnaire, the Insomnia Severity Index Sleep Survey, the Epworth Sleepiness Scale, the Patient Global Impression of Change scale, Clinical Global Impression scale, or the Medical Outcomes Study-Sleep Scale,. In some embodiments, improvement of symptoms relating to ALS- or PLS-related spasticity, muscle spasms, and muscle cramps are improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more upon treatment with a composition of the present invention, e.g., as measured by cramp frequency and/or cramp severity as assessed by a Visual Analogue Scale or Numerical Rating Scale for Pain, the Modified Ashworth Scale, the Tardieu Scale, a Spasticity Numerical Rating Scale, the ALS Assessment Questionnaire, the Insomnia Severity Index Sleep Survey, the Epworth Sleepiness Scale, the Patient Global Impression of Change scale, Clinical Global Impression scale, or the Medical Outcomes Study-Sleep Scale,.

In some embodiments, the subject has been diagnosed with or identified as having night cramps (also known as nocturnal cramps). Night cramps are spontaneous muscle contractions that occur during sleep, can be very painful, and often recur throughout the night. Elderly people, e.g., over 50 years of age, are at higher risk for experiencing night cramps. In some embodiments, the night cramps occur in the leg, foot, toe, or back. In some embodiments, the night cramps occur in the leg or foot. In some embodiments, the night cramps may disrupt the sleep of the subject. In some embodiments, improvement of symptoms relating to night cramps may be measured by cramp frequency, cramp severity, cramp free nights, and/or cramp-free days, and/or as assessed by a Visual Analogue Scale, a Numerical Rating Scale for Pain, the Clinical Global Impression of Change (CGI-C) Scale, or Patient Global Impression of Change (PGI-C) Scale. In some embodiments, improvement of symptoms relating to night cramps are improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more upon treatment with a composition of the present invention, e.g., as measured by a Visual Analogue Scale, a Numerical Rating Scale for Pain, the Clinical Global Impression of Change (CGI-C) Scale, or Patient Global Impression of Change (PGI-C) Scale.

In one embodiment, the subject has been diagnosed with or identified as having spinal cord spasticity. Spasticity is the uncontrolled tightening or contracting of the muscles that is common in individuals with spinal cord injuries and a variety of nervous system diseases. Spasticity is usually defined as a velocity-dependent increase in the tonic stretch reflex (muscle tone) with exaggerated tendon jerks, clonus, and spasms, resulting from the hyperexcitability of the stretch reflex. About 65%-78% of the spinal cord injury population have some amount of spasticity, and it is more common in cervical (neck) than thoracic (chest) and lumbar (lower back) injuries. In one embodiment, the subject has experienced a central nervous system injury, such as a brain injury, a stroke, or a traumatic spinal cord injury. For example, the central nervous system injury is associated with unwanted or abnormal muscle contractions or spasms, or the absence of normal muscle contractions.

In some embodiments, the subject has been diagnosed or identified as experiencing muscle cramps, spasms, dystonias, or fasciculations (e.g., unwanted or abnormal muscle cramps, spasms, dystonias, or fasciculations). Muscle cramps, spasms, dystonias, or fasciculations can also occur as a consequence of other diseases or disorders, such as diabetes (e.g., diabetic neuropathy), Addison's disease, peripheral artery disease, hypertension, alcoholism, liver cirrhosis, renal failure, hypothyroidism, neuromuscular diseases (e.g., amyotrophic lateral sclerosis, primary lateral sclerosis, progressive muscular atrophy, progressive bulbar palsy, pseudobulbar palsy, spinal muscular atrophy, progressive spinobulbar muscular atrophy (e.g., Kennedy's disease), or post-polio syndrome), and metabolic disorders (e.g., adrenoleukodystrophy, phenylketonuria, or Krabbe disease). In some embodiments, the subject experiences muscle cramps associated with renal dialysis. Accordingly, the methods described herein are also useful for treating or evaluating subjects that have been diagnosed with the other diseases or disorders associated with muscle cramps described herein.

Muscle cramps, spasms, dystonias, or fasciculations can occur as a side effect of some drugs. Medications that can cause muscle cramps include: diuretics, oral contraceptives, blood pressure medications. The methods described herein can also be useful to treat or evaluate subjects that are prescribed or take medication that cause muscle cramps. Exemplary medications that can induce muscle cramps include, but are not limited to: diuretics, e.g., Lasix (furosemide), Microzide (hydrochlorothiazide); Alzheimer's disease medication, e.g., Aricept (donepezil); myasthenia gravis medication, e.g., Prostigmine (neostigmine); cardiovascular medication, e.g., Procardia (nifedipine); osteoporosis medication, e.g., Evista (raloxifene); asthma medication, e.g., Brethine (terbutaline), Proventil and Ventolin (albuterol); Parkinson's disease medication, e.g.,Tasmar (tolcapone); cholesterol medication, e.g., statins such as Crestor (rosuvastatin), Lescol (fluvastatin), Lipitor (atorvastatin), Mevacor (lovastatin), Pravachol (pravastatin), or Zocor (simvastatin).

In an embodiment, the compositions and methods disclosed herein are suitable for treating or evaluating a subject that has an absence of a normal muscle contraction, such as a gait abnormality. Gait abnormalities are deviations from normal walking or unusual and uncontrollable walking patterns. Examples of gait abnormalities include propulsive gait, scissors gait, spastic gait, steppage gait, and waddling gait. For example, the gait abnormality is “foot drop,” in which the dropping of the forefoot happens due to muscular weakness, damage to nerves, or paralysis of muscles. Gait abnormalities are often associated with neuromuscular diseases or disorders. In some embodiments, improvement of symptoms relating to a gait abnormality may be measured by the Timed 25-Foot Walk Test. In some embodiments, improvement of symptoms relating to a gait abnormality are improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more upon treatment with a composition of the present invention, e.g., as measured by the Timed 25-Foot Walk Test.

### Neurological Conditions

A neurological condition includes a disease, disorder, or condition of the brain, spine, and nerves, and comprises peripheral nervous system conditions and central nervous system conditions.

### Peripheral Nervous System (PNS) Conditions

Compositions of the invention can be used to treat conditions affecting the peripheral nervous system (PNS). These conditions include: diseases, disorders, or injuries to the peripheral nervous system include but are not limited to: cramp fasciculation syndrome, Isaacs' Syndrome or neuromyotonia (NMT), peripheral neuropathy (e.g., diabetic neuropathy), carpal tunnel syndrome, or EBV infection. Other peripheral nervous system diseases and conditions include but are not limited to: amyloid neuropathies, diabetic neuropathies, nerve compression syndromes, peripheral nervous system neoplasms, a peripheral nerve hyperexcitability disorder (e.g., neuromyotonia, benign fasciculation syndrome, or cramp fasciculation syndrome), brachial plexux neuropathies, Guillain-Barre syndrome, neuralgia, polyneuropathies, complex regional pain syndromes, mononeuropathies, neuritis, acrodynia, neurofibromatosis, hand-arm vibration syndrome, pain insensitivity, and Tarlov cysts. In some embodiments, improvement of symptoms relating to a peripheral nervous condition (e.g., a peripheral nerve hyperexcitability disorder) may be measured by the Numerical Rating Scale, the Modified Ashworth Scale, the Patient Global Impression of Change Scale, or the Clinical Global Impression Scale. In some embodiments, improvement of symptoms relating to a peripheral nervous condition (e.g., a peripheral nerve hyperexcitability disorder) are improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more upon treatment with a composition of the present invention, e.g., as measured by the ALS Assessment Questionnaire, the Numerical Rating Scale, the Modified Ashworth Scale, the Patient Global Impression of Change Scale, or the Clinical Global Impression Scale.

### Central Nervous System (CNS) Conditions

The compositions of the invention can be used to treat conditions affecting the central nervous system (CNS). These conditions include: diseases, disorders, and injuries to the central nervous system due to tumor, multiple sclerosis, spasticity due to cerebral palsy, stroke, a motor neuron disease (e.g., amyotrophic lateral sclerosis, primary lateral sclerosis, progressive muscular atrophy, progressive bulbar palsy, or psuedobulbar palsy), spinal injury or stenosis. There are many other central nervous system diseases and conditions, including infections of the central nervous system such as encephalitis and poliomyelitis, early-onset neurological disorders including ADHD and autism, late-onset neurodegenerative diseases such as Alzheimer's disease, Parkinson's disease, and essential tremor, autoimmune and inflammatory diseases such as multiple sclerosis and acute disseminated encephalomyelitis, genetic disorders such as Krabbe's disease and Huntington's disease, as well as amyotrophic lateral sclerosis and adrenoleukodystrophy. Other diseases of the CNS include but are not limited to: catalepsy, epilepsy, meningitis, migraine, tropical spastic paraparesis, arachnoid cysts, locked-in syndrome, and Tourette's syndrome. Anxiety disorders may also be characterized as a CNS condition. Anxiety disorders can be classified into: generalized anxiety disorder, phobic disorders, panic disorders, agoraphobia, social anxiety disorder, obsessive-compulsive disorder, post-traumatic stress disorder, separation anxiety, and situational anxiety. In some embodiments, improvement of symptoms relating to a central nervous condition (e.g., a motor neuron disease) may be measured by the ALS Assessment Questionnaire, the Numerical Rating Scale, the Modified Ashworth Scale, the Patient Global Impression of Change Scale, or the Clinical Global Impression Scale. In some embodiments, improvement of symptoms relating to a central nervous condition (e.g., a motor neuron disease) are improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more upon treatment with a composition of the present invention, e.g., as measured by the ALS Assessment Questionnaire, the Numerical Rating Scale, the Modified Ashworth Scale, the Patient Global Impression of Change Scale, or the Clinical Global Impression Scale.

### Tactile Sensitivity

The compositions of the invention may be useful in treating tactile sensitivity or tactile defensiveness (TD). TD refers to patterns of observable behavioral and emotional responses which are aversive, negative, and out of proportion to certain types of tactile stimuli that are often found by most people to be non-painful. TD is a sensory integrative dysfunction in which the brain is unable to process and use information through the senses. Tactile sensitivity can result from conditions such as autism, dyspraxia, neuralgia, panic or anxiety disorders, or from venomous bites or stings.

Electrolyte Imbalance and/or Vitamin Deficiency

The compositions of the invention may be useful in treating conditions associated with an electrolyte imbalance and/or vitamin deficiency. Examples of such condition include, but are not limited to: hyponatremia, hypernatremia, hyperkalemia, hypokalemia, hypercalcemia, hypocalcemia, hyperchloremia, hypochloremia, hypermagnesemia, hypomagnesemia, hyperphosphatemia, hypophosphatemia, kidney disease, rickets, scurvy, beriberi, pellagra, calcium deficiency, eating disorders, vitamin D deficiency, vitamin A deficiency, biotin deficiency, ariboflavinosis, vitamin K deficiencies, hypocobalaminemia, paraesthesia, night blindness, magnesium or thiamine deficiency, hypoparathyroidism, medullary cystic disease, and adrenocortical carcinoma. In some embodiments, the compositions of the invention may be useful as a weight control or weight loss agent, e.g., as a satiety-promoting agent.

### Connective Tissue Diseases

The compositions of the invention are also useful for treating connective tissue diseases. Examples of diseases include but are not limited to: degenerative joint disease (DJD), marfan syndrome, Ehlers-Danlos syndrome, osteogenesis imperfect, Stickler syndrome, Alport syndrome, congenital contractural arachnodactyly, psoriatic arthritis, systemic lupus erythematosus, rheumatoid arthritis, scleroderma, Sjögren's syndrome, and mixed connective tissue disease.

### Throat Conditions

The compositions of the invention can also treat throat disorders or throat injuries (e.g., from chemicals, cancer, surgery, or infection). Examples of throat disorders include, but are not limited to acid reflux, tonsillitis, pharyngitis, laryngospasm due to throat surgery, laryngitis, dysphagia, and spasmodic dysphonias.

### Sarcoidosis

The compositions of the invention are also useful for treating sarcoidosis. Sarcoidosis is a disease involving abnormal collections of inflammatory cells (granulomas) that can form as nodules in multiple organs. The granulomas are most often located in the lungs or its associated lymph nodes, but any organ can be affected. The compositions of the invention are useful in treating various types of sarcoidosis including but not limited to: annular sarcoidosis, erythrodermic sarcoidosis, ichthyosiform sarcoidosis, hypopigmented sarcoidosis, Lofgren syndrome, lupus pernio, morpheaform sarcoidosis, mucosal sarcoidosis, neurosarcoidosis, papular sarcoid, scar sarcoid, subcutaneous sarcoidosis, systemic sarcoidosis, and ulcerative sarcoidosis.

### Respiratory Conditions

The compositions of the invention may also useful for treating a respiratory condition or illness. Respiratory conditions involve the organs and/or tissues involved in respiration, including the lungs, trachea, bronchi, bronchioles, alveoli, pleura, pleural cavities. Without wishing to be bound by theory, activation of the TRPA1 and/or TRPV1 ion channels through administration of an ion channel activator (e.g., a TRPV1 channel activator, TRPA1 channel activator, ASIC channel activator, or combinations thereof) of the present invention may affect airway sensory nerve reactivity, thus leading to bronchodilation of airway smooth muscle. Exemplary respiratory conditions for which the composition of the present invention may be useful in treating include, but are not limited to, asthma, chronic obstructive pulmonary disease, bronchitis, emphysema, pneumonia, cystic fibrosis, pleural cavity diseases, influenza, or cold.

### Cough and Hiccup

The compositions of the present invention may also be useful for treating or reducing cough in a subject. Cough is a reflex that is often repetitive in nature and may aid in clearing the breathing passages from particles, irritants, secretions, and the like. Coughing may be voluntary or involuntary. Exemplary conditions related to cough include respiratory conditions (e.g., asthma, chronic obstructive pulmonary disease, bronchitis, emphysema, pneumonia, cystic fibrosis, pleural cavity diseases, influenza, or a cold), exposure to allergens or chemical irritants, or inflammation. In some embodiments, the composition of the present invention may be a cough suppressant.

The compositions of the present invention may also be useful for treating or reducing the severity of hiccup in a subject. Hiccup is a sudden involuntary spasm of the diaphragm and respiratory muscles and organs that results in a sudden closure of the glottis. Irritation of the vocal cords, throat, trachea, and nerves that extend from the head to the neck may result in hiccup. Hiccups may persist in an otherwise healthy subject, or may be a symptom of another disease or disorder. Exemplary conditions related to hiccup include gastrointestinal conditions (e.g., indigestion or acid reflux), respiratory conditions (e.g., asthma, chronic obstructive pulmonary disease, bronchitis, emphysema, pneumonia, cystic fibrosis, pleural cavity diseases, influenza, or a cold), stroke, brain seizure, nerve damage (e.g., damage to the vagus or phrenic nerve), brain tumor or other neurological condition, meningitis, encephalitis, pneumonia, inflammation, renal failure, anxiety, stress, or exposure to allergens or chemical irritants. In some embodiments, the composition of the present invention may be a hiccup reliever or suppressant.

### Anxiety Disorders

In some embodiments, the subject has been diagnosed with or identified as having an anxiety disorder. An anxiety disorder may be characterized as a condition involving the central nervous system and can cause feelings of fear, anxiety, and anguish in the subject. These conditions can result in unwanted or abnormal muscle cramps, spasms, dystonias, and fasciculations that may be treated by compositions of the disclosed invention. Exemplary anxiety disorders include generalized anxiety disorder, phobic disorders, panic disorders, agoraphobia, social anxiety disorder, obsessive-compulsive disorder, post-traumatic stress disorder, separation anxiety, and situational anxiety.

The symptoms of any of the above-identified diseases or disorders may be prevented or improved by administration of a compound of the present invention (e.g., a gingerol (e.g., 6-gingerol), a shogaol (e.g., 6-shogaol), a capsaicinoid (e.g., capsaicin), e.g., wherein the composition is substantially free of a related analog thereof. In some embodiments, the prevention or improvement of symptoms relating to any of the above-identified diseases or disorders may be measured by a particular endpoint test known to one of skill in the art, e.g., the ALS Assessment Questionnaire, the Numerical Rating Scale, the Modified Ashworth Scale, the Patient Global Impression of Change Scale, the Clinical Global Impression Scale, the Toronto Western Spasmodic Torticollis Rating Scale, the Tsui score, the Oropharyngeal Swallow Efficiency Test, the visual analogue scale, the Insomnia Severity Index Sleep Survey, the Epworth Sleepiness Scale, or another similar test, scale, survey, or standard. In some embodiments, symptoms relating to any of the above-identified diseases or disorders are improved by about 1%, about 2%, about 3%, about 4%, about 5%, about 10%, about 15%, about 20%, about 25%, about 30%, about 35%, about 40%, about 45%, about 50%, about 60%, about 70%, about 80%, about 90%, about 95%, or more upon treatment with a composition of the present invention, e.g., as measured by any of the above-identified endpoint tests, scales, surveys, or standards.

### Combination Therapies

In certain embodiments, additional therapeutic agent(s) may be administered with compositions of the present invention for, e.g., the treatment of peripheral nervous system conditions (e.g., peripheral neuropathy), central nervous system conditions, muscle conditions and disorders (e.g., fibromyalgia, muscle spasms and cramps (e.g., nocturnal cramps), painful muscle contractions (e.g., a muscle contraction of the head or neck), neuromuscular disorders (e.g., motor neuron disease) or dystonia (e.g., cervical dystonia, blepharospasm, back spasms, or leg cramps due to spinal stenosis)), connective tissue diseases (e.g., degenerative joint disease), throat conditions (e.g., dysphagia or spasmodic dysphonias), tactile sensitivity, electrolyte imbalance and/or vitamin deficiency, respiratory conditions (e.g., asthma), cough, hiccup, and sarcoidosis. In one embodiment, the candidate therapeutic agents are agents already known in the art for use for other conditions or disorders, e.g., neuromuscular therapeutic agents. When combination therapy is employed, the additional therapeutic agent(s) can be administered as a separate formulation or may be combined with any of the compositions described herein.

For example, any of the compositions described herein can be used for the treatment of nocturnal (or night) cramps. In some embodiments, the compositions can be used in combination with a sleep aid. Sleep aids that can be used in combination with the compositions and methods described herein include: antihistamines (e.g., diphenhydramine and doxylamine); benzodiazepines (e.g., estazolam (ProSom), flurazepam (Dalmane), quazepam (Doral), temazepam (Restoril), and triazolam (Halcion)); non-benzodiazepine sedative hypnotics (e.g., eszopiclone (Lunesta), zalepon (Sonata), and zolpidem (Ambien)); and melatonin receptor agonist hypnotics (e.g., ramelteon (Rozerem). Still other sleep aids that can be used in combination with the compositions and methods described herein include: chamomile, valerian root, kava kava, lemon balm, passionflower, lavender, St. John's Wort, melatonin, tryptophan (e.g., L-tryptophan), 5-hydroxytryptophan (5-HTP), catnip, hops, rhodiola, oatstraw, lavender, GABA, L-theanine, linden, ginseng (e.g., Siberian ginseng), honey, nutmeg, mugwort, butterbur, rauwolfia, taumelloolch, American hellebore, quassia, tulip tree, brewer's yeast, inositol, skullcap, phosphatidylserine, calcium, magnesium, vitamin B6, vitamin B12, and pantothenic acid (B5).

In another embodiment, any of the compositions described herein can be used for the treatment of painful muscle contraction of the head or neck as in tension, cluster or migraine headache. In some embodiments, the compositions can be used with analgesics, including aspirin, ibruprofen, acetaminophen, or naproxen; with triptans including sumatriptan, rizatriptan, naratriptan; with mild sedatives including butalbital; with anti-depressants including amitriptyline; with dihydroergotamine mesylate; or with ketorolac.

Any of the compositions described herein can be also be used for the treatment of focal dystonia. In some embodiments, the compositions can be used with botulinum toxin; with anticholinergic agents including trihexyphenidyl and benztropine; with GABAergic agents including benzodiazepines; and with dopaminergic agents including tetrabenazine and levodopa.

In further embodiments, the compositions described herein can be also be used for the treatment of muscle claudication pain due to inactivity or restriction as seen in “economy class syndrome”, paralysis, peripheral artery disease or immobilization. In some embodiments, the compositions can be used with cilostazol or with pentoxifylline. The compositions described herein can be also be used for the treatment of sarcoidosis. In some embodiments, the compositions can be used with non-steroidal anti-inflammatory drugs (NSAIDs) including ibuprofen and aspirin; with corticosteroids, including prednisone and prednisolone; and with steroid-sparing agents, including azathioprine, methotrexate, mycophenolic acid, and leflunomide.

In other embodiments, any of the compositions described herein can also be used in combination with a treatment for pain or a disorder relating to the oral cavity, such as oral lesions, canker sores, cold sores, thrush, gingivitis, leukoplakia, halitosis, or dry mouth. In some embodiments, the composition can be used with or antibacterial or antiviral agents to treat or prevent tooth decay or carries.

In other embodiments, any of the compositions described herein can also be used in combination with a treatment for pain or a disorder relating to the stomach or gastrointestinal tract, such as indigestion, heartburn, colitis, irritable bowel syndrome, constipation, diarrhea, lactose intolerance, gastroesophageal reflux disease, ulcers, nausea, or stomach cramps. In some embodiments, the compositions can be used with antacids (e.g., simethicone, magaldrate, aluminum salts, calcium salts, sodium salts, magnesium salts, alginic acid) laxatives, H₂ antagonists (e.g., ranitidine, famotidine, nizatidine, cimetidine) or proton pump inhibitors (e.g., omeprazole, lansoprazole, esomeprazole, dexlansoprazole, rabeprazole, or pentoprazole), and antidiarrheals (e.g., bismuth subsalicylate).

In other embodiments, any of the compositions described herein can be also be used for the treatment of disease, disorder or injury to the peripheral nervous system such as cramp fasciculation syndrome, peripheral neuropathy, carpal tunnel syndrome or EBV. In some embodiments, the compositions can be used to treat cramp fasciculation syndrome with β-blockers; analgesics including ibuprofen and acetaminophen; magnesium; or carbamazepine. In some embodiments, the compositions can be used to treat peripheral neuropathy with tricyclic antidepressants, including amitriptyline; with antiepileptic therapies including gabapentin and sodium valproate; with synthetic cannabinoids including nabilone; with pregabalin; or with serotonin-norepinephrine reuptake inhibitors (SNRIs), including duloxetine. In some embodiments, the compositions can be used to treat carpal tunnel syndrome with corticosteroids.

### EXAMPLES

### Example 1

### Stimulation of TRPV1 and TRPA1 Channels in Doral Root Ganglion Neurons by Exemplary Ccompositions

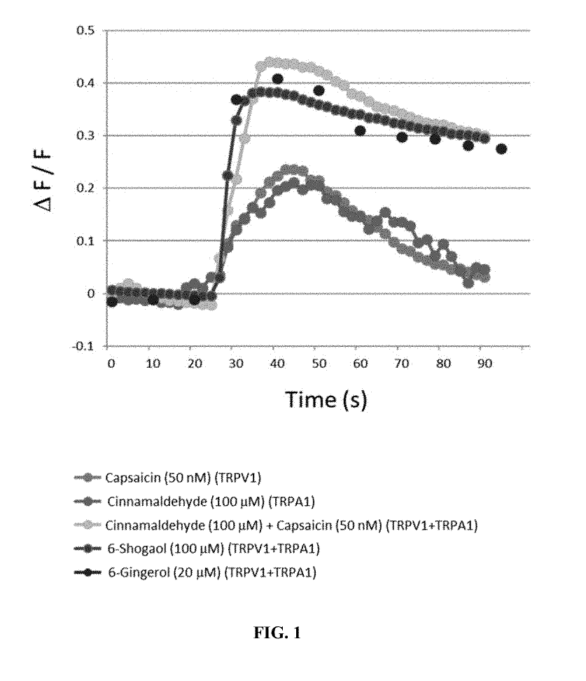

Selective TRPV1 and TRPA1 activation responses were measured for exemplary compositions of the invention in human dorsal root ganglion neurons ex vivo and compared with TRPV1/TRPA1 co-activation responses. The degree of neuron activation was assessed by inward calcium flux using neurons preloaded with a calcium-sensitive fluorescent dye. Intracellular cellular calcium levels were then monitored by wide-field fluorescent microscopy. The change in intracellular fluorescence relative to baseline fluorescence (ΔF/F) was quantified, and the results are shown in FIG. 1. Only in the case of co-activation of both TRP channels was a supra-additive or synergistic response observed. 6-shogaol and 6-gingerol, both of which activate TRPA1 and TRPV1, yields a synergistic response that mimics the response obtained by a combination of the selective TRPV1 (capsaicin) and TRPA1 (cinnamaldehyde) activators.

### Example 2

### Efficacy of Single Dose Oral Administration of Exemplary Compositions in Prevention of Externally-Induced Cramping of the Flexor Hallucis Brevis Muscle

### Study Objectives and Design

The goal of this study was to assess the effectiveness of multiple exemplary formulations of the present invention in prevention of externally-induced cramping of the flexor hallucis brevis muscle of the foot. The study is a single-center, single-blind, vehicle-controlled, 8-period, fixed sequence single administration study. Screening of subjects occurred within 28 days, with each subject treated over 9 consecutive days beginning with a Cramp Familiarization Day (Day −1) and followed directly by an 8-period fixed sequence treatment phase (Days 1-8).

Up to sixteen healthy adult subjects between the ages 19-65 were selected for inclusion in the study. The subjects were screened to ensure that application of an external electrical stimulus to the flexor hallucis brevis muscle of the foot resulted in induction of a cramp in the hallux (i.e., big toe) on Day −1. On Day 1, the subjects began the fixed sequence treatment phase, involving consumption of a beverage formulation, and then assessment for cramp induction at three distinct time points (prior to treatment, 1 hour, and 2 hours).

### Study Procedure

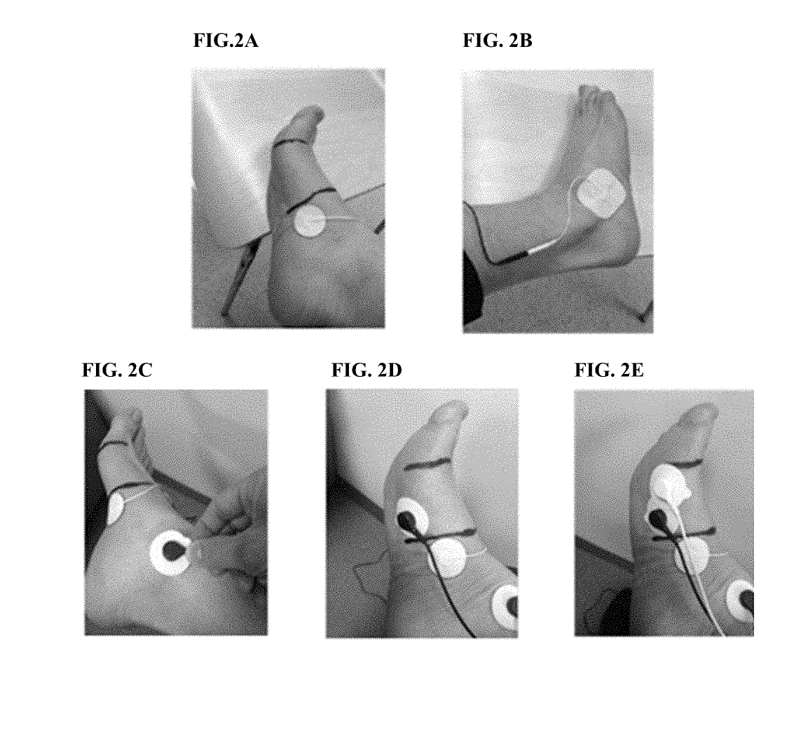

The stimulation protocol is similar to that described by Minetto and Botter (Minetto, M. A. and Botter, A. Muscle Nerve (2009) 40:535-544). The subject was comfortably seated with their leg supported and relaxed, and the testing area of the foot was cleaned with isopropyl alcohol (91%). Stimulating electrodes were placed on the flexor brevis muscle on the upper arch of the foot in the region of the medial plantar nerve (FIG. 2A), as well as below the lateral malleolus of the ankle (FIG. 2B). The EMG recording electrodes were placed over the belly of the flexor hallucis brevis muscle, with a first reference electrode at the medial malleolus of the ankle (FIG. 2C), a second reference electrode at the base of the flexor hallucis brevis muscle (FIG. 2D), and a third reference electrode toward the big toe on the widest region of the flexor hallucis brevis muscle (FIG. 2E). Correct placement of the both the stimulating and reference electrodes was confirmed by direct stimulation to the foot. The subject was then instructed to briefly contract his toes in an isometric fashion in order to provide a reference maximum EMG signal, after which the electrodes and leads were secured in place by rip-tape and sports pro-wrap.

Baseline muscle cramp induction was then induced through electrical stimulation by the stimulator (EMS7500 stimulator, Koalaty Products), delivering repetitive stimuli lasting for a total of 7 seconds each. The EMG signal was monitored and recorded for each stimulus. If the flexor hallucis muscle relaxed directly after a stimulus, no cramp was found to be induced. Additional stimuli were applied at increasing increments of 2 Hz until cramp induction. Induction of a cramp in the flexor hallucis brevis muscle resulted in strong downward deflection of the first tow after stimulation ceased, accompanied by a strong root mean signal (˜100-130) that slowly returned to baseline upon relaxation of the muscle. Upon cramp induction, an additional stimulus was applied at an increased stimulation rate of 2 Hz to ensure a durable cramp. Each cramp stimulation session was monitored for roughly 5 minutes, from which the duration of the cramp was determined as the time the EMG readings were maintained above 3 standard deviation increments greater than the mean EMG baseline reading prior to stimulation.

After baseline cramp induction, the subject was administered one of the beverage formulations of the study (i.e., one of eight exemplary compositions of the present invention as described in Table 1). After administration of the beverage formulation, cramp induction was then carried out at 1 hour and 2 hours post-consumption as described above, using the same intensity and rate setting determined in the initial baseline analysis. On Day −1, no beverage formulation was administered. On Days 1-8, one of each of the beverage formulations summarized in Table 1 was administered to the subjects. Subjects were monitored for serious adverse events (SAEs) and unexpected adverse events (UAEs) after administration of each beverage formulation and cramp stimulation.

TABLE 1

| | | Formulation | Treatment |
|---|---|---|---|
| Treatment | Active Components | Base | Volume (mL) |
| A | capsicum extract, ginger | 1 | 50 |
| | extract, cinnamon extract | | |
| B | capsicum extract, ginger | 2 | 60 |
| | extract, cinnamon extract | | |
| C | capsicum extract and ginger | 3 | 50 |
| | extract | | |
| D | 6-gingerol (38 mg) and | 4 | 50 |
| | capsaicin (0.2 mg) | | |
| E | 6-shogaol (38 mg) | 4 | 50 |
| F | 6-shogaol (38 mg), capsaicin | 4 | 50 |
| | (0.2 mg), and trans- | | |
| | cinnamaldehyde (170 mg) | | |
| G | 6-shogaol (38 mg) and | 4 | 50 |
| | capsaicin (0.2 mg) | | |
| H | Vehicle | — | 50 |

Formulation Base #1: light karo syrup and distilled H₂O (1:1 ratio)
Formulation Base #2: organic lime juice concentrate, organic cane sugar, sodium benzoate, potassium sorbate, salt, pectin, organic compliant honey lime flavor, distilled H₂O.
Formulation Base #3: sodium saccharine (0.1%), sorbitol (38.5%), carboxymethylcellulose sodium (0.5%), citric acid (0.2%), glycerin (25%), sodium citrate (0.3%), distilled H₂O.
Formulation Base #4: light karo syrup and distilled H₂O (1:1 ratio), ethanol

An additional study was carried out following the procedures outlined above in which the treatments summarized in Table 2 were administered.

TABLE 2

| | | Formulation | Treatment |
|---|---|---|---|
| Treatment | Active Components | Base | Volume (mL) |
| I | capsicum extract, ginger | 1 | 50 |
| | extract, cinnamon extract | | |
| J | 6-gingerol (38 mg) | 3 | 50 |
| K | 6-shogaol (76 mg) | 3 | 50 |
| L | 6-gingerol (76 mg) | 3 | 50 |
| M | 6-gingerol (38 mg) and | 3 | 50 |
| | capsaicin (0.2 mg) | | |
| N | 6-shogaol (38 mg) and | 3 | 50 |
| | capsaicin (0.2 mg) | | |
| O | 6-shogaol (38 mg) and | 3 | 25 |
| | capsaicin (0.2 mg) | | |
| P | Vehicle | — | 50 |

### Data Analysis and Results

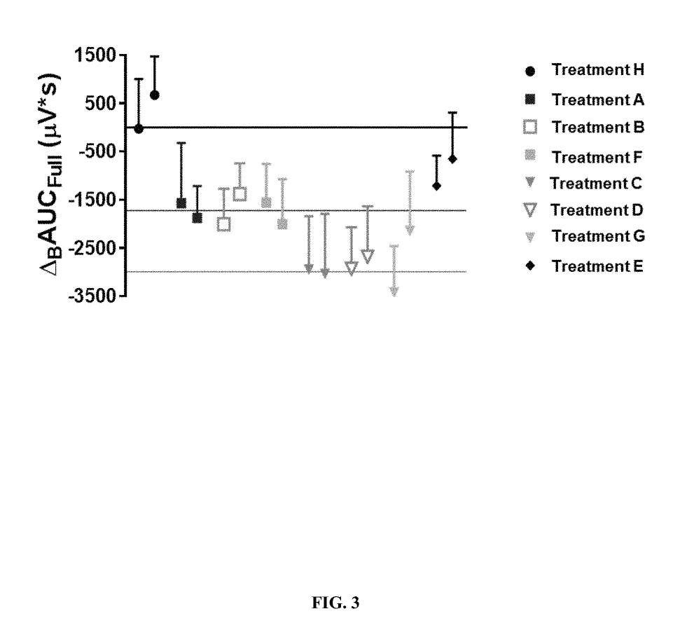

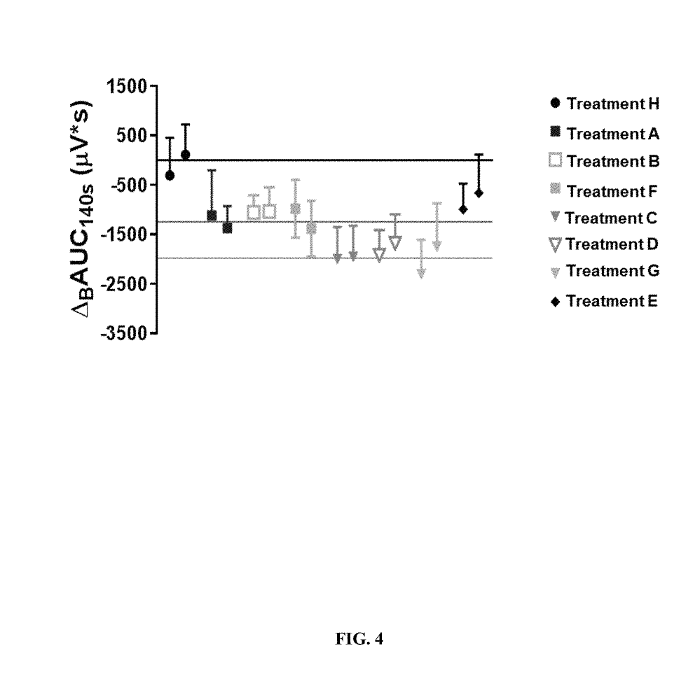

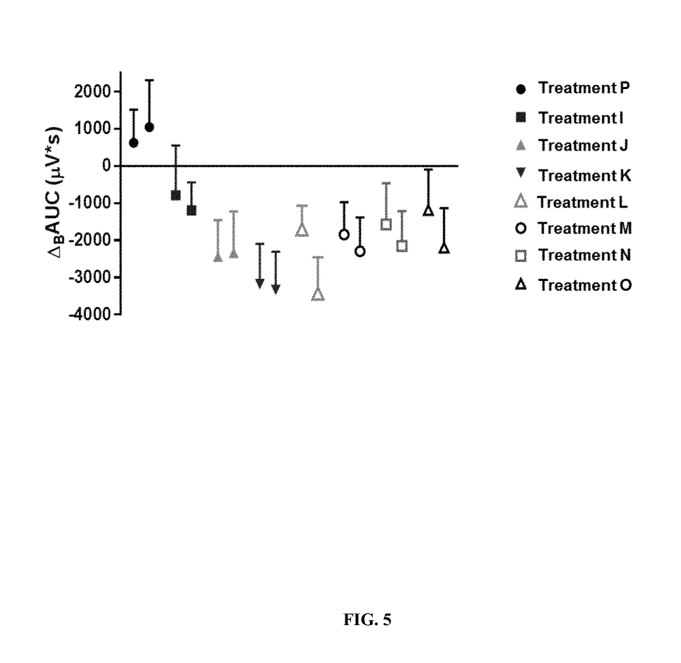

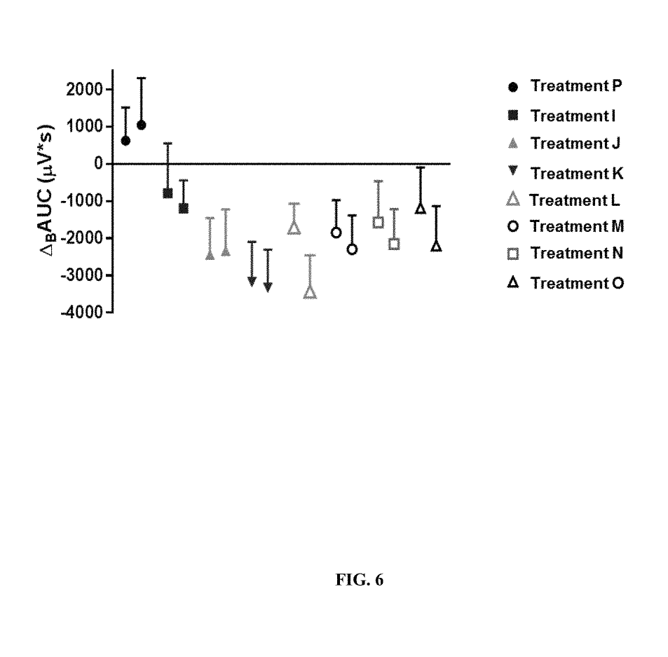

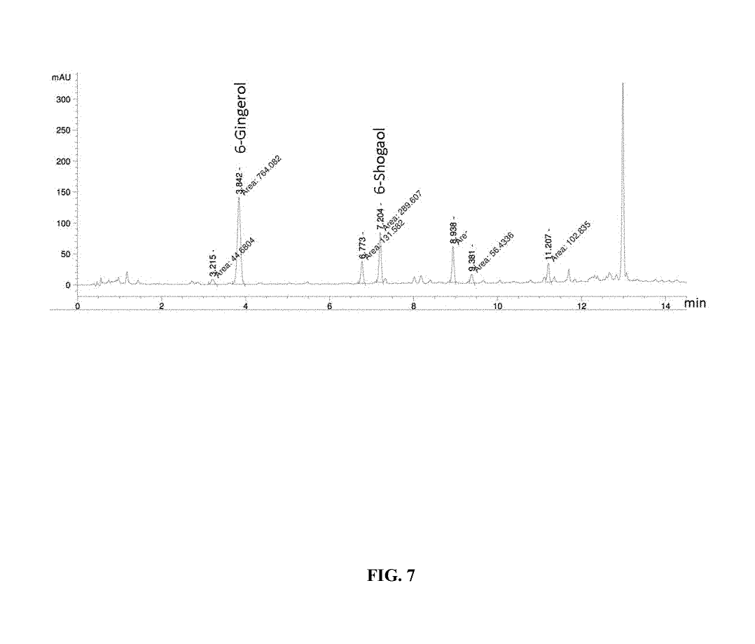

The collected EMG data was analyzed for the baseline adjusted integrated EMG (IEMG) level, also referred to as area under the curve (AUC), and cramp duration. The IEMG level was calculated as the measured EMG reading during the cramp minus the pre-cramp readings; this value was integrated over the duration of the cramp. The resulting IEMG was used as a quantitative index of cramping, which was then normalized to the baseline (i.e., pre-dose) value from that day for each time point for each subject. The cramp duration (seconds) was compared across the vehicle and administered treatments and across the multiple time points. Results were further analyzed using ANOVA and subject to appropriate statistical analyses. FIG. 3 and FIG. 5 summarize the average change in AUC values for all subjects compared by administered treatment in Tables 1 and 2. As shown, administration of each of Treatments A-G and I-0 resulted in a larger Δ_{B} value than was observed for Treatment H (vehicle), suggesting that each treatment was efficacious in lessening the intensity of the muscle cramp (p=0.0006). FIG. 4 and FIG. 6 depict the average change in AUC values at a 140 second Δ_{AUC} for all subjects compared by administered treatment. The data mirror the overall results shown in FIG. 3 and FIG. 6 respectively, and further support the finding that each treatment was shown to have an effect on reducing muscle cramp intensity (p=0.0006).

### Example 3

### Efficacy and Analysis of Exemplary Compositions for Use in Decreasing Intensity of Cramping of the Flexor Hallucis Brevis Muscle

The parental extract used to prepare Treatment A was analyzed by HPLC in order to identify the key active ingredients in the treatment (FIG. 7). Peak retention times were compared to known molecules present in the natural extracts used to prepare the mixture, and it was found that 6-shogaol and 6-gingerol shared HPLC elution profiles with two known molecules. In comparison to 6-gingerol, 6-shogaol was a minor component of Treatment A.

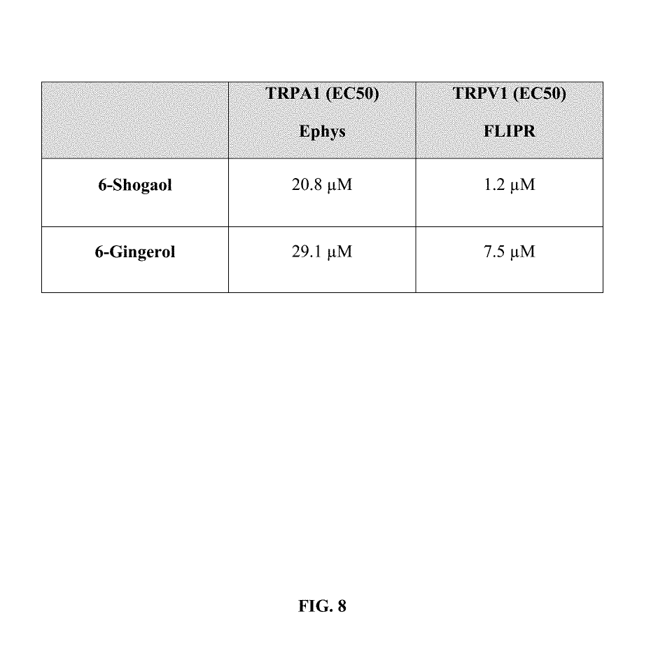

Both 6-shogaol and 6-gingerol were assayed for the ability to activate either the TRPA1 or the TRPV1 ion channel through the determination of the respective EC₅₀ values (FIG. 8). The EC₅₀ values of 6-shogaol and 6-gingerol for TRPA1 were determined by automated Patch-clamp (Patchliner®, Nanion Technologies GmbH) assay in HEK cells stably transfected with human TRPA1. The EC₅₀ values of 6-shogaol and 6-gingerol for TRPV1 were measured by monitoring Ca²⁺ flux in CHO cells stably transfected with human TRPV1 pre-loaded with a calcium-sensitive fluorescent dye (FLIPR). As summarized in FIG. 8, both molecules were confirmed as agonists of TRPA1 and TRPV1, displaying similar potencies for each ion channel.

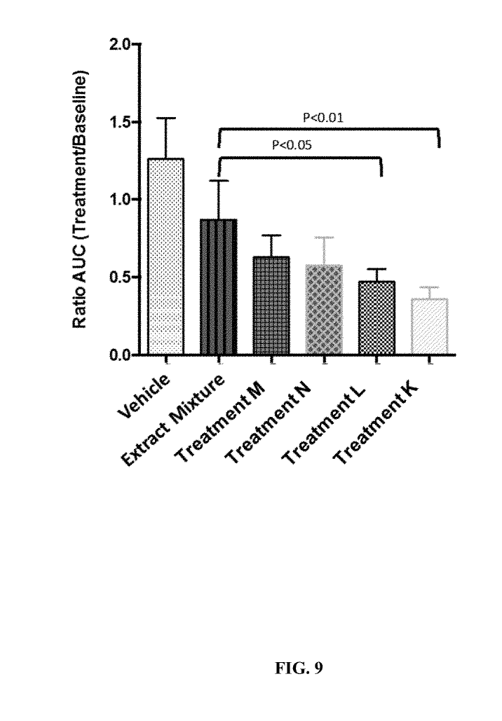

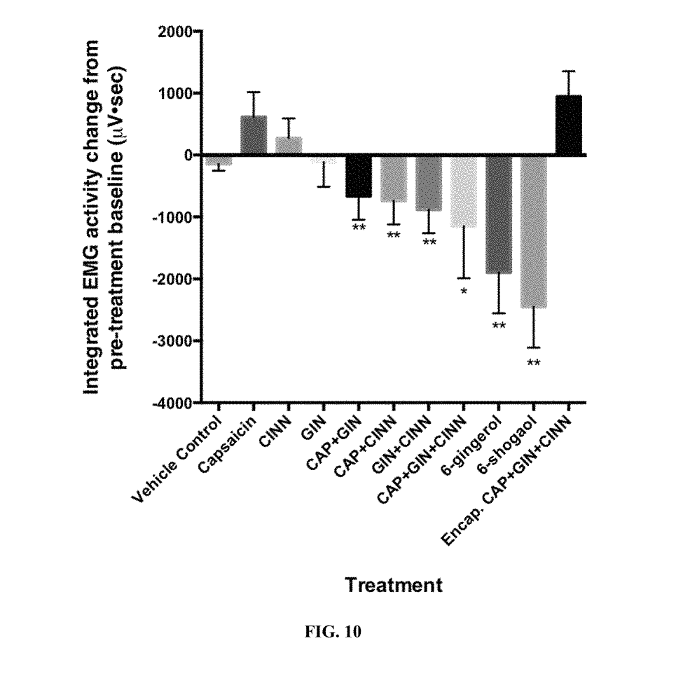

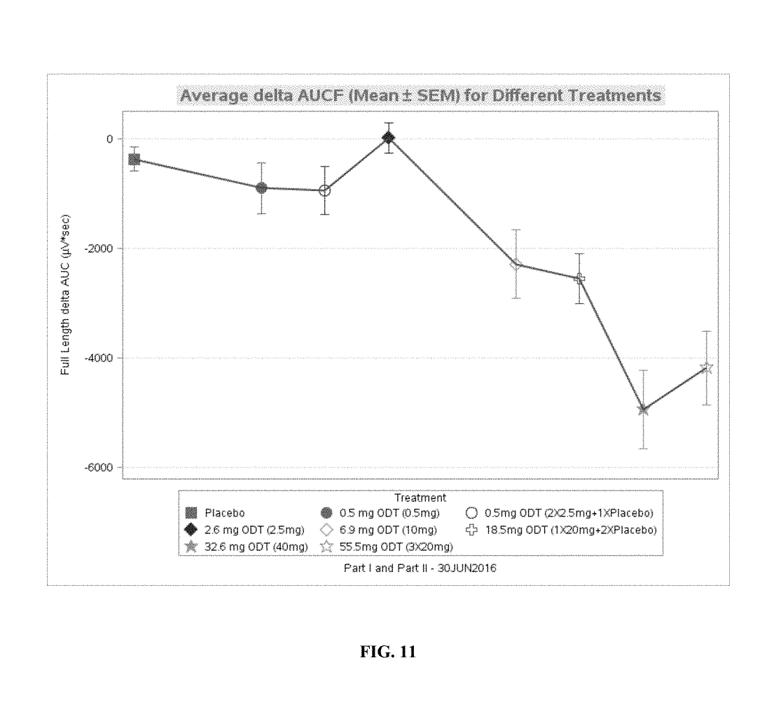

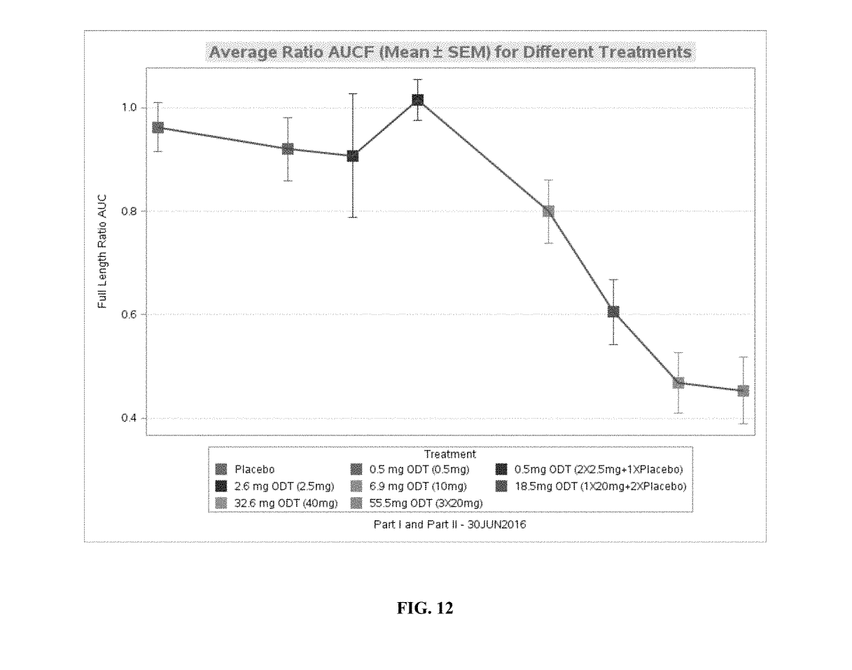

In order to gauge the efficacy of 6-shogaol and 6-gingerol and combinations thereof in treating muscle cramps, a study similar to that outlined in Example 1 was undertaken. 6-shogaol and 6-gingerol were synthesized under GMP conditions and provided to normal, healthy volunteers (n=9). As described in Example 1, cramps were electrically elicited in the flexor hallucis brevis muscle 1 hour prior to treatment, followed by measurements 1 hour and 2 hours after dosing. The ratio of the AUC values derived from the pre-treatment cramp baseline relative to the post-treatment cramp were calculated, as was the mean AUC ratio derived across the 1 hour and 2 hour timepoints (FIG. 9). Using a generalized linear mixed model (Turkey-Kramer post-hoc), Treatment K, Treatment L, Treatment N, and Treatment M treatment arms were found to be significantly different from the vehicle control (p<0.01). Both Treatment K and Treatment L demonstrated improved efficacy at decreasing cramp intensity compared to the parental extract formulation (Treatment A) by at least 2-fold (FIG. 9), suggesting that single agent compounds may likely be responsible for efficacy in the mixture.

An additional study was carried out comparing exemplary compounds and combinations thereof as summarized in Table 3 below, each administered as a beverage formulation.

TABLE 3

| | | | Treatment Volume |
|---|---|---|---|
| Active Components | Amounts | | (mL) |
| Vehicle | NA | | 50 |
| capsaicin | 0.0036 | g | 50 |
| cinnamon extract | 2.5 | g | 50 |
| ginger extract | 3.75 | g | 50 |
| capsicum extract + ginger | 0.076 | g (capsicum) | 50 |
| extract | 1.5 | g (ginger) | |
| capsicum extract + | 0.076 | g (capsicum) | 50 |
| cinnamon extract | 1.0 | g (cinnamon) | |
| ginger extract + cinnamon | 1.5 | g (ginger) | 50 |

Cramp intensity was assessed by integrating the area under the curve (AUC) of a surface EMG signal over a 140 second observation period post electrical induction of a cramp in the flexor hallucis brevis muscle as previously described. The effect of treatment was calculated based upon the AUC change from a pre-treatment baseline cramp, wherein greater efficacy resulted in greater negative AUC differences. The overall change between the pre-treatment baseline and mean vehicle control response was minimal. 6-shogaol (29 mg), a single molecule that activates both TRPA1 and TRPV1, afforded a greater decrease in cramp intensity compared to other treatments tested, leading to a 5-fold reduction in AUC compared to its study specific vehicle control. The results are shown in FIG. 10. Treatments that demonstrated a significant difference from study-specific vehicle controls are annotated with an asterisk (* p<0.05, ** p<0.01).

### Example 4

### Efficacy of Exemplary Orally Disintegrating Tablet in Prevention of Externally-Induced Cramping of the Flexor Hallucis Brevis Muscle

### Study Objectives and Design

The goal of this study was to assess the effectiveness of 6-shogaol formulated as an orally disintegrating tablet (ODT) in the prevention of externally-induced cramping of the flexor hallucis brevis muscle of the foot. The study was carried in two parts. The first part was an open label, placebo-controlled, 5-visit study evaluating vehicle (lactose and sucrose), 0.5 mg, 2.5 mg, 10/6.9 mg, and 40/33 mg 6-shogaol. The second part was a randomized, single-blind, placebo-controlled, 4-visit study evaluating vehicle (lactose and sucrose), 0.5 mg, 20/18.5 mg, and 60/55.5 mg 6-shogaol. Screening of subjects occurred within 28 days in a similar manner as described above in Example 1. Note that dosage values separated by a “/” indicate the nominal versus the actual doses as determined by Chromadex.

### Data Analysis and Results

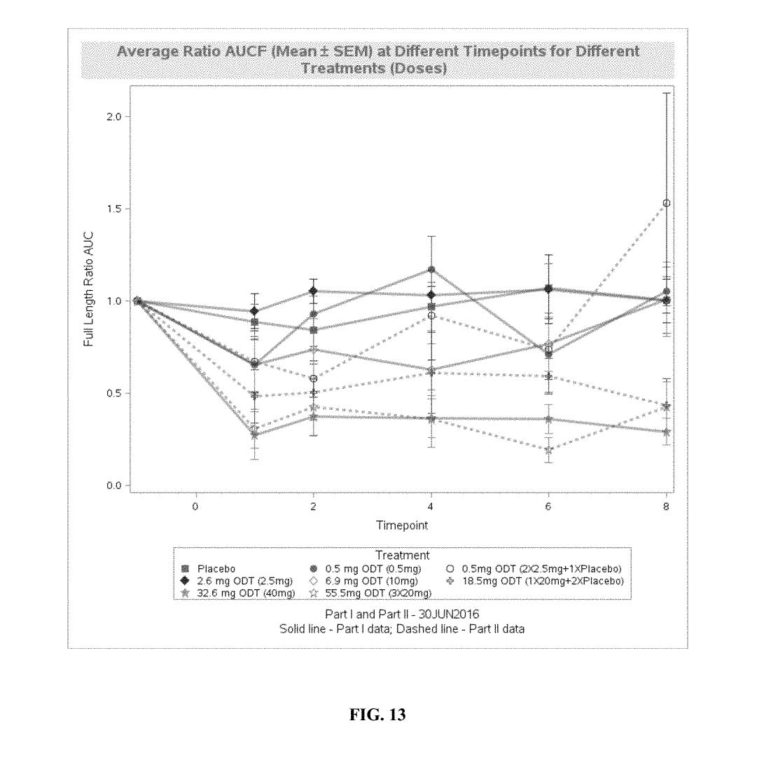

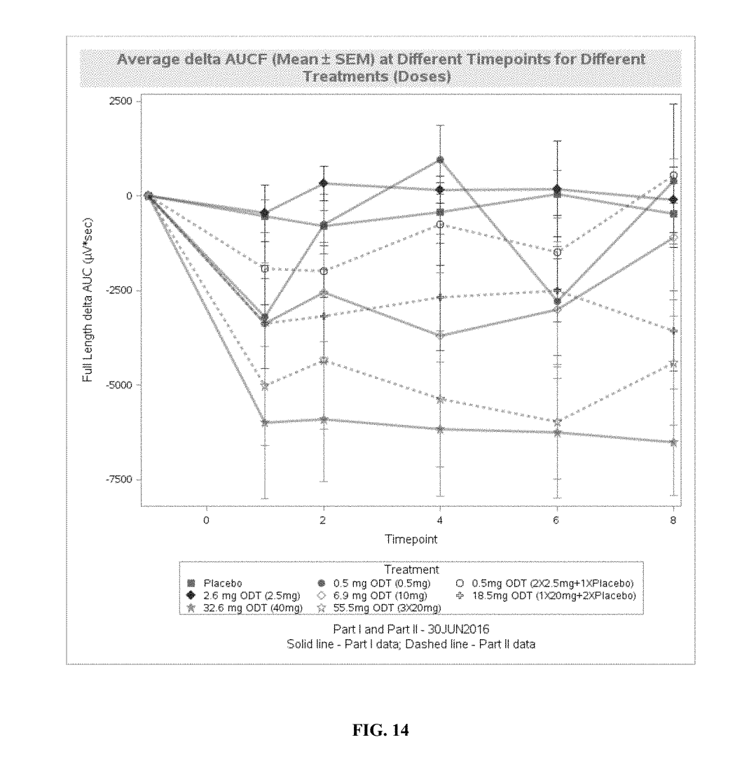

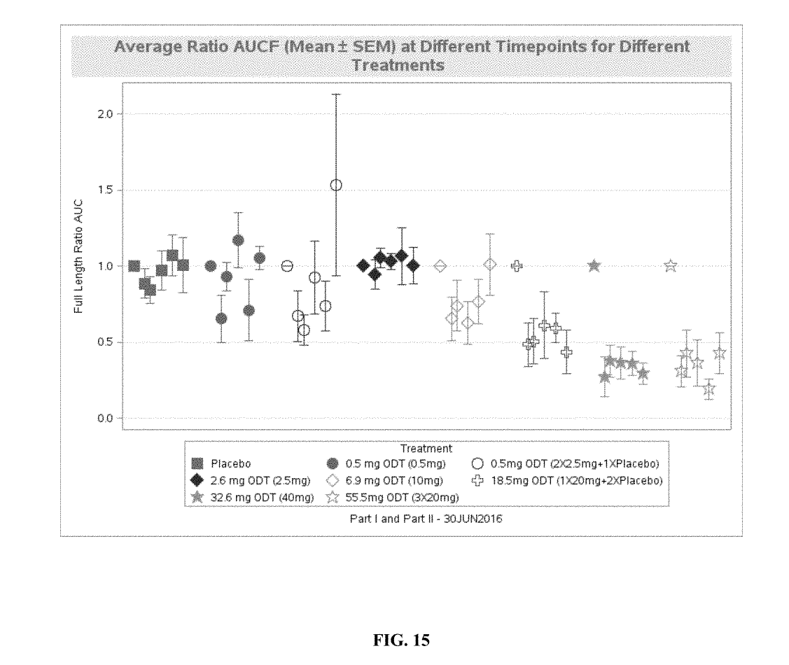

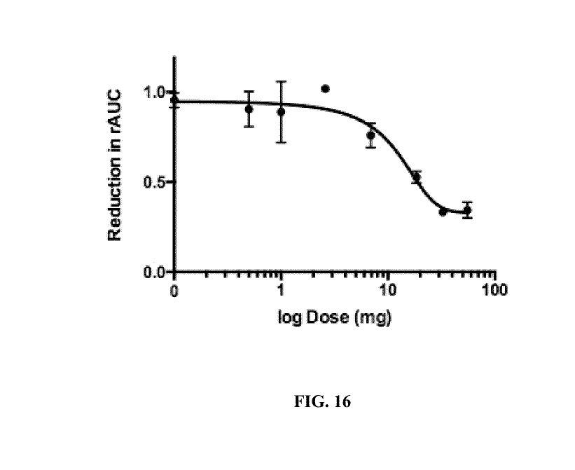

The collected EMG data was analyzed for the baseline adjusted integrated EMG (IEMG) level, also referred to as area under the curve (AUC), and cramp duration, as described in Example 1. FIG. 11 and FIG. 12 summarize the average change and ratio in AUC values for all subjects compared by administered treatment both parts of the study. As shown, administration of each treatment A AUCF value than was observed for the vehicle, suggesting that each treatment was efficacious in lessening the intensity of the muscle cramp. FIG. 13 and FIG. 14 depict the average change in AUC values over time compared by dosage in both study parts, and indicate that while no efficacy was achieved at the 0.5 mg and 2.6 mg dosage levels, roughly 40% cramp inhibition was observed at 6.9 mg 6-shogaol and 75% cramp inhibition was observed at the 33 mg and 55 mg dosage levels, both of which were sustained over 8 hours. The data in FIG. 15 and FIG. 16 further support these findings.

### Example 5

### Efficacy and Tolerability of a 6-Shogaol Orally Disintegrating Tablet to Treat Nocturnal Leg Cramps

The goal of this study is to assess the effectiveness and tolerability of 6-shogaol formulated as an orally disintegrating tablet (ODT) in the prevention and treatment of nocturnal leg cramps (NLC) over time. The study is a multi-center, two-part, two-periods per part, randomized, double-blind, placebo-controlled, cross-over study to evaluate the effects of the 6-shogaol ODT formulation compared with an ODT control and an ODT-placebo. Subjects were evaluated for inclusion or exclusion criteria during the screening period (up to 28 days). Eligible subjects, about 80 male and females, entered the study and commence the Run-in Period 1, a 14 day period during which all subjects received placebo capsules. The purpose of this period is to remove potential placebo responders. Placebo responders, or subjects with scores of 1 or 2 on the Clinical Global Impression of Change (CGI-C), were excluded from entering the cross-over parts of the trial (i.e., Periods 2, 3, 4, and 5).

Part I will assess the 6-shogaol ODT (up to 20 mg) versus ODT-placebo. After completion of Period 1, subjects who meet entry eligibility criteria will be randomized to one of two possible treatment consequences, either ODT-placebo—6-shogaol ODT (up to 20 mg) or 6-shogaol ODT (up to 20 mg)—ODT-placebo. Subjects will be allocated to each treatment in a 1:1 ratio. Each cross-over period (Periods 2 and 3) is 21 days. Subjects will return to the clinic on day 15 of each cross-over period and on day 22, wherein assessments of efficacy and tolerability will be made.

Part II will assess the 6-shogaol ODT (up to 30 mg) versus ODT-control. There will be approximately 7 to 14 days between Part I and Part II. Those subjects who complete Part I and agree to continue to Part II of the study will return to the clinic after a 7 to 14 days washout. Subjects will then be randomized to one of two possible treatment sequences: ODT-control—6-shogaol ODT (up to 30 mg) or 6-shogaol ODT (up to 30 mg) or ODT-control. Subjects will be allocated to each treatment sequence in a 1:1 ratio. Each cross-over period (Periods 4 and 5) is 42 days. Subjects will return to the clinic every two weeks, on Days 15, 29, and 43 of each cross-over period, when assessments of efficacy and tolerability will be made.

Telephone contact will be made with each subject on Day 7 (+/−2) in each period to assess adverse events and compliance and 7 days (+/−4 days) post last study product administration to assess adverse events. The criteria for evaluation of efficacy for the treatment of NLC will include assessment of the following parameters: a) cramp-free nights; b) cramp frequency; c) cramp pain/intensity' d) Patient Global Impression of Change (PGI-C); e) CGI-C; and f) Medical Outcomes Study Sleep Survey (MOS-SS). The criteria for evaluation of safety and tolerability of the 6-shogaol ODT will include assessment of: a) adverse events; b) clinical laboratory tests; c) vital signs; d) physical exam; and e) electrocardiogram.

### Example 6

### Efficacy and Tolerability of a 6-Shogaol Beverage to Treat Multiple Sclerosis Spasticity and Spasms/Cramps

The goal of this study is to assess the effectiveness and tolerability of 6-shogaol formulated as a beverage in the treatment of multiple sclerosis (MS). The study is a multi-center, randomized, blinded, placebo-controlled, cross-over study to evaluate the effects of a 6-shogaol beverage in subjects with symptoms of spasticity and spasms/cramps due to MS. Subjects were evaluated for inclusion or exclusion criteria during the screening period (up to 14 days). Eligible subjects will include about 60 male and females that are 18 years of age or older that have symptoms of spasticity and spasms/cramps due to MS. These subjects entered the study and commence the Run-in Period 1, a 14 day period during which all subjects will receive placebo capsules. The purpose of this period is to remove potential placebo responders. Placebo responders, or subjects with scores of 1 or 2 on the Clinical Global Impression of Change (CGI-C) and spasticity scores lower than 24 on the last 6 daily Numerical Rating Scale (NRS), were excluded from entering the cross-over parts of the trial (i.e., Periods 2 and 3).

After completion of Period 1, subjects who meet entry eligibility criteria will be randomized to one of two possible treatment consequences, either inactive control-6-shogaol beverage or 6-shogaol beverage—inactive control). Subjects will be allocated to each treatment in a 1:1 ratio. Each cross-over period (Periods 2 and 3) is 14 days. There will be a 7-day washout period between Periods 2 and 3. Subjects will return to the clinic at the end of each cross-over period, wherein assessments of efficacy and tolerability will be made.

Telephone contact will be made with each subject on Day 7 (+/−2) in each period to assess adverse events and compliance and 7 days (+/−4 days) post last study product administration to assess adverse events. Assessment of the efficacy and tolerability of the 6-shogaol beverage for the treatment of spasticity and spasms/cramps due to MS will be determined by the following parameters: a) Modified Ashworth Scale (MAS); b) Tardieu Scale (TS); c) Numerical Rating Scale (NRS); d) Barthel Activities of Daily Living (ADL); e) Timed 25-Foot Walk (T25-FW); f) Clinical Global Impression (CGI); g) Quality of Life (QoL) questionnaires; and h) Insomnia Severity Index (ISI) Sleep Survey.

### Example 7

### Efficacy and Tolerability of a 6-Shogaol Orally Disintegrating Tablet to Treat Motor Neuron Disease

The goal of this study is to assess the effectiveness and tolerability of 6-shogaol formulated as an orally disintegrating table (ODT) on muscle cramps, spasticity, and sleep in subjects that have motor neuron disease. The study is a randomized, blinded, cross-over study to evaluate the efficacy and tolerability of a 6-shogaol ODT comprising up to about 20 mg of 6-shogaol. Subjects were evaluated for inclusion or exclusion criteria and asked to take the study products during the screening visit. Eligible subjects will include about 50 people diagnosed with amyotrophic lateral sclerosis (ALS) or progressive lateral sclerosis (PLS) for at least 12 months, spasticity of at least 3 months duration that is not completely relieved by current therapy, and experience an estimated 6- or more cramps per month or greater than 15 cramps per week. These subjects entered the study and commence the Run-in Period, a 14 day period during which all subjects received complete a daily telephone questionnaire through an interactive voice response system to document the previous day's cramps (including number of, time, duration, location, and pain/intensity level) as well as to obtain information on the subjects' spasticity through the measurement of the Numerical Rating Scale (NRS). The purpose of this Run-in period is to obtain baseline information on the muscle cramps experienced by each subject and the level of spasticity and sleep duration.

After completion of the Run-in Period, subjects will be randomized to one of two possible treatment consequences, either inactive control-6-shogaol ODT or 6-shogaol ODT—inactive control). Subjects will be allocated to each treatment in a 1:1 ratio. Subjects will be instructed to take the study product two times a day. Each cross-over period (Periods 1 and 2) is 14 days. There will be a 7-day washout period between Periods 1 and 2. During Periods 1 and 2, subjects will be again asked to complete a daily telephone questionnaire to document the same information as during the Run-in Period, as well as to confirm study product compliance. Subjects will return to the clinic on Period 2 Day 14 for the End of Study Visit. Assessments and surveys will be completed at each clinic visit. Assessment of the efficacy of the 6-shogaol ODT for the treatment of spasticity, cramps, and sleep disturbance due to motor neuron disease will be determined by the following parameters: a) pain and intensity of cramps; b) Insomnia Severity Index (ISI) Sleep Survey; c) ALS Assessment Questionnaire (ALSAQ); d) Numerical Rating Scale (NRS) for spasticity; e) Modified Ashworth Scale (MAS); f) Tardieu Scale (TS); g) Patient Global Impression of Change (PGI-C) scale; h) Clinical Global Impression of Change (CGI-C) scale; i) adverse events; j) laboratory evaluations; and k) vital signs.

### Equivalents

The disclosures of each and every patent, patent application, and publication cited herein are hereby incorporated herein by reference in their entirety. While this disclosure has been described with reference to specific aspects, it is apparent that other aspects and variations may be devised by others skilled in the art without departing from the true spirit and scope of the disclosure. The appended claims are intended to be construed to include all such aspects and equivalent variations. Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this disclosure has been particularly shown and described with references to preferred embodiments thereof, it will be understood by those skilled in the art that various changes in form and details may be made therein without departing from the scope of the disclosure encompassed by the appended claims.

What is claimed is:

1. A method of treating a subject diagnosed with amyotrophic lateral sclerosis (ALS), primary lateral sclerosis (PLS), or progressive muscular atrophy (PMA), the method comprising orally administering to the subject an effective amount of a composition comprising 6-shogaol, wherein the composition is substantially free of an analog of 6-shogaol.

2. The method of claim 1, wherein the treating comprises improving a symptom of ALS, PLS, or PMA.

3. The method of claim 2, wherein the symptom comprises muscle cramp, muscle spasm, muscle spasticity, muscle pain, or muscle soreness.

4. The method of claim 3, wherein the improving the symptom comprises reduced cramp frequency, reduced cramp severity, increased cramp free nights, increased cramp-free days, reduced cramp duration, reduced sleep disruption, faster walking speed, reduced spasm frequency and severity, reduced frequency of spasticity, reduced muscle soreness, reduced fasciculation severity, reduced fasciculation frequency, reduced pain severity, and/or improved respiratory conditions.

5. The method of claim 4, wherein the improving the symptom is measured by: visual analogue scale, a numerical rating scale for pain or spasticity, clinical global impression of change (CGI-C) scale, patient global impression of change (PGI-C) scale, Modified Ashworth Scale (MAS), ALS Assessment Questionnaire (ALSAQ), Tardieu Scale, Insomnia Severity Index Sleep Survey, and/or Epworth Sleepiness Scale.

6. The method of claim 3, wherein the symptom occurs in a skeletal muscle of the subject.

7. The method of claim 4, wherein the improving the symptom comprises reducing the symptom by about 10% -about 90% relative to the baseline.

8. The method of claim 7, wherein the improving the symptom comprises reducing muscle cramp frequency and/or duration by about 10% -about 90% relative to the baseline.

9. The method of claim 7, wherein the improving the symptom comprises reducing muscle pain by about 10%-about 70% relative to the baseline.

10. The method of claim 7, wherein the improving the symptom comprises reducing muscle spasticity or spasm by about 10%-about 90% relative to the baseline.

11. The method of claim 10, wherein the reducing muscle spasticity or spasm is measured by the Patient Global Impression of Change scale, the Modified Ashworth Scale, and/or the Tardieu Scale.

12. The method of claim 1, wherein composition is formulated as an orally disintegrating tablet (ODT).

13. The method of claim 12, wherein the ODT comprises about 0.5 mg-about 100 mg of the 6-shogaol.

14. The method of claim 13, wherein between about 15 mg-about 40 mg of the 6-shogaol is administered to the subject.

15. The method of claim 14, wherein the 6-shogaol is administered to the subject once, twice, or three times daily.

16. An oral dosage composition comprising about 0.5 mg to about 100 mg of 6-shogaol, wherein the composition has a residence time of greater than about 10 seconds in the mouth of a subject; and wherein the composition is substantially free of an analog of 6-shogaol.

17. The oral dosage composition of claim 16, wherein the composition further comprises one or more pharmaceutically acceptable excipient.

18. The oral dosage composition of claim 17, wherein the composition comprises about 15 mg-about 60 mg of the 6-shogaol.

19. The oral dosage composition of claim 18, wherein the composition is formulated as a single dosage unit or a multiple dosage units.

20. An orally disintegrating tablet (ODT) comprising about 0.5 mg to about 100 mg of 6-shogaol, and one or more pharmaceutically acceptable excipient, wherein the composition is substantially free of an analog of 6-shogaol.

21. The ODT of claim 20, wherein the ODT has a residence time of greater than about 10 seconds in the mouth of a subject.

22. The ODT of claim 20, wherein the ODT comprises about 15 mg-about 60 mg of the 6-shogaol.

23. The ODT of claim 22, wherein the ODT is formulated as a single dosage unit or a multiple dosage units.

24. A method of achieving a therapeutic response or stabilization of a subject diagnosed with amyotrophic lateral sclerosis (ALS) or primary lateral sclerosis (PLS), the method comprising orally administering to the subject an effective amount of a composition comprising 6-shogaol, wherein the composition is substantially free of an analog of 6-shogaol; wherein the administering results in an improved symptom in the subject, selected from: reduced cramp frequency, reduced cramp severity, increased cramp free nights, increased cramp-free days, reduced muscle spasm, reduced muscle spasticity, reduced muscle pain, reduced muscle soreness, reduced cramp duration, reduced sleep disruption, faster walking speed, reduced spasm frequency and severity, reduced frequency of spasticity, reduced fasciculation severity, reduced fasciculation frequency, reduced pain severity, and/or improved respiratory conditions.

25. The method of claim 24, wherein the achieving the therapeutic response or stabilization of the subject is assessed by: visual analogue scale, a numerical rating scale for pain or spasticity, clinical global impression of change (CGI-C) scale, patient global impression of change (PGI-C) scale, Modified Ashworth Scale (MAS), ALS Assessment Questionnaire (ALSAQ), Tardieu Scale, Insomnia Severity Index Sleep Survey, and/or Epworth Sleepiness Scale.

26. The method of claim 25, wherein the achieving the therapeutic response or stabilization of the subject comprises increase or reduction of the symptom by about 10%-about 90% relative to the baseline.

27. The method of claim 24, wherein composition is formulated as an orally disintegrating tablet (ODT).

28. The method of claim 27, wherein the ODT comprises about 0.5 mg-about 100 mg of the 6-shogaol.

29. The method of claim 28, wherein between about 15 mg-about 40 mg of the 6-shogaol is administered to the subject.

30. The method of claim 29, wherein the 6-shogaol is administered to the subject once, twice, or three times daily.
